(19) United States
(12) Patent Application Publication
(10) Pub. No.: US 2005/0260754 A1
(43) Pub. Date: Nov. 24, 2005

(54) Constructs and methods for the regulation of gene expression

(76) Inventors: Michael Kock, Schifferstadt (DE); Jorg Bauer, Ludwigshafen (DE)

Correspondence Address: MORRISON & FOERSTER LLP, 1650 TYSONS BOULEVARD, SUITE 300, MCLEAN, VA, 22102, US

(21) Appl. No.: 10/508,263

(22) PCT Filed: Mar. 17, 2003
(86) PCT No.: PCT/EP03/02735

§ 371 Date: Sep. 20, 2004

(30) Foreign Application Priority Data
Mar. 20, 2002 (DE) 102 12 892.8

(51) Int. Cl. C12N 15/85; C07H 21/02

(52) U.S. Cl. 435/455; 536/23.1

(57) ABSTRACT

The present invention relates to constructs and methods for regulating the gene expression of at least two endogenous target genes by introducing, into a eukaryotic cell or a eukaryotic organism, an at least partially double-stranded ribonucleic acid molecule, the ribonucleic acid molecule comprising at least two ribonucleotide sequence segments which are homologous to various genes of the eukaryotic cell.

The present invention relates to constructs and methods for regulating the gene expression of at least two endogenous target genes by introducing, into a eukaryotic cell or a eukaryotic organism, an at least partially double-stranded ribonucleic acid molecule, the ribonucleic acid molecule comprising at least two ribonucleotide sequence segments which are homologous to various genes of the eukaryotic cell.

The specific inhibition of the gene expression of defined genes is one of those technologies of biotechnology which have been the subject of the most intense research. In this context, the expression of antisense RNA is the most widely used approach and described extensively (EP-A1 0 458 367; EP-A1 0 140 308; van der Krol A R et al. (1988) BioTechniques 6(10):658-676; de Lange P et al. (1995) Curr Top Microbiol Immunol 197:57-75, inter alia). However, antisense-RNA-mediated approaches have the disadvantage that stoichiometric amounts of the antisense RNA are required to bring about an effective inhibition of the target mRNA. Further problems are connected with the introduction, into the cells, of sufficient amounts of the antisense RNA and with the instability of the antisense. RNA. Antisense-RNA-based approaches are therefore inefficient in most cases.

A further approach for gene regulation is “co-suppression” meaning the reduction of the expression of an endogenous target gene by the recombinant expression of a sense RNA of this target gene (EP-A1 0 465 572). Co-suppression is believed to be based on more than one mechanism. The disadvantage is the lacking reliability and reproducibility of the method. In some cases, suppression takes place, while in other cases—caused by the expression of the sense RNA—the expected overexpression takes place. Also, the resulting phenotype is frequently not stable. The application of co-suppression is essentially limited to plants.

Various modifications of the methods based on antisense RNA or cosuppression are known. Thus, WO 93/23551 describes a method for inhibiting a plurality of genes by expressing a chimeric antisense RNA or sense RNA. The method cannot solve the usual problems connected with antisense RNA or sense RNA and remains inefficient.

WO 98/36083 and WO 99/15682 describe the regulation of gene expression by means of viral expression systems (“virus induced gene silencing” VIGS).

WO 99/32619 and WO 99/53050 describe methods for inhibiting individual target genes using an RNA with double-stranded structure, where the target gene and the region of the RNA duplex have at least partial identity with one another (see also: Montgomery MK et al. (1998) Proc Natl Acad Sci USA 95:15502-15507; Sharp P A (1999) Genes & Development 13(2):139-141; Fire A et al. (1998) Nature 391:806-11). The method is currently also referred to as “RNA interference” (RNAi) and its mechanism and action resembles the abovementioned VIGS method.

While the above-described methods, in particular the RNAi method, solve some of the problems in connection with the reduction of individual target genes, no satisfactory solution has been provided to date for other problems, in particular for the parallel suppression of a plurality of target genes. A large number of approaches in biotechnology require not only the reduction of an individual gene, but of a plurality of target genes, such as, for example, various genes of one or more metabolic pathways or whole gene families. To date, this could only be achieved with laborious and time-consuming approaches. The approaches frequently required the individual regulation of the individual target genes by successive transformation, for example using different expression constructs, each of which encoded an antisense RNA of a target gene. In addition to the fact that this is a considerably laborious and time-consuming approach, there is the disadvantage that only a limited number of selection markers, suitable promoters and the like is available for many systems and organisms, which makes multiple transformations considerably more difficult and requires for example the deletion of the markers after the transformation and selection. Frequently, the multiple use of a promoter has undesired consequences such as, for example, epigenetic gene silencing. Here, the multiple use of the control sequences leads to their inactivation, similar to the above-described cosuppression.

It is an object of the present invention to provide novel methods which make possible an efficient reduction of the expression, in a eukaryotic cell or a eukaryotic organism, of at least two endogenous target genes. We have found that this object is achieved by the present invention.

In a first aspect, the invention relates to a method for reducing the expression of at least two different endogenous target genes in a eukaryotic cell or a eukaryotic organism by introducing, into said eukaryotic cell or said eukaryotic organism, an at least partially double-stranded ribonucleic acid molecule, the double-stranded ribonucleic acid molecule comprising
a) at least two “sense” ribonucleotide sequences, where in each case at least one of these “sense” ribonucleotide sequences is essentially identical to at least one part of the “sense” RNA transcript of each of said endogenous target genes and
b) “antisense” ribonucleotide sequences which are essentially complementary to said “sense” ribonucleotide sequences of a).

A further aspect of the invention comprises an at least partially double-stranded ribonucleic acid molecule, where the double-stranded ribonucleic acid molecule comprises
a) at least two “sense” ribonucleotide sequences, where in each case at least one of these “sense” ribonucleotide sequences is essentially identical to at least one part of the “sense” RNA transcript of an endogenous target gene, but where not all “sense” ribonucleotide sequences are identical to the “sense” RNA transcript of a single endogenous target gene, and
b) “antisense” ribonucleotide sequences which are essentially complementary to said “sense” ribonucleotide sequences of a).

A further aspect is the use of the double-stranded ribonucleic acid molecule according to the invention in one of the methods according to the invention.

The present invention overcomes the problems set out above and makes possible a rapid, particularly effective method for regulating the expression of various target genes. This results in particular in the following advantages:
a) Transgenic organisms or cells in which more than one target gene is inhibited can be generated in a single transformation step.
b) The transcription rate for each ribonucleotide sequence of the dsRNA is the same. This prevents multiple phenotypes caused by differing expression levels, as are obtained frequently when separate ribonucleotide sequences are expressed individually—for example caused by the differing insertion site in the genome. This advantage ensures the same level of inhibition of all target genes and drastically reduces the selection steps required for generating an organism in which all target genes are suppressed efficiently.
c) Economical use of control elements such as promoters and selection markers is made possible. Moreover, problems as can arise upon the multiple use of a particular control element, in particular a promoter (“epigenic gene silencing”), do not arise.
d) Segregation of the individual ribonucleotide sequences in subsequent breeding and hybridization steps, as is necessary in the case when a plurality of expression constructs are used, is prevented. This substantially facilitates and accelerates the subsequent breeding of stable lines.
e) Effective gene suppression is made possible in organisms with complex genomes, for example polyploid genomes, such as, for example, some plants. Owing to the large number of copies of individual genes, these organisms are not suitable for traditional mutagenesis and selection methods.

Surprisingly, no troublesome interference between the individual ribonucleotide sequence segments was observed in the method according to the invention.

“Endogenous target gene of a eukaryotic cell or a eukaryotic organism” refers to any nucleic acid sequence in a eukaryotic cell, a eukaryotic organism or in a part, organ, tissue, seed and the like of same which is capable of transcription. This may take the form of naturally occurring or else artificially introduced sequences (such as, for example, transgenic sequences), with naturally occurring sequences being preferred. Naturally occurring sequences are preferred and comprise not only the homologous sequences of the eukaryotic cell or the eukaryotic organism, but also genes of pathogens which are present in the eukaryotic cell or the eukaryotic organism following infection by a pathogen. The target gene can be located in the chromosomal DNA or the DNA of the organelles (such as, for example, of the plastids, for example chloroplasts and the like) or else be located extrachromosomally in the cell. The naturally occurring, homologous sequences of the eukaryotic organism preferably comprise genes of same which are present in the genome in a stable manner, the term genome referring to the totality of the genetic information and comprising both the chromosomal and the plastid DNA. The endogenous target gene is preferably a gene which naturally occurs in the chromosomal DNA. Preferred genes are those whose reduced expression brings about a modified phenotype.

“Reduction of” or “to reduce” the expression of a target gene is to be understood in the broad sense in the present context and comprises the partial or essentially complete prevention or blocking of the expression of the target gene or the RNA, mRNA, rRNA, tRNA derived therefrom and/or of the protein product encoded by it in a cell or organism or a part, tissue, organ, cell or seed derived therefrom, which prevention or blockage is based on differing cell-biological mechanisms. A reduction for the purposes of the invention comprises the quantitative reduction of an RNA, mRNA, rRNA, tRNA expressed by the target gene and/or of the protein product encoded by it up to an essentially complete absence of these. In this context, the expression of a particular RNA, mRNA, rRNA, tRNA and/or of the protein product encoded by it, in a cell or an organism, is preferably reduced by more than 50%, especially preferably more than 80%, very especially preferably more than 90%, most preferably more than 95%, in comparison with the same cell or organism which has not been subjected to the method. In this context, the reduction can be determined by methods with which the skilled worker is familiar. Thus, the reduction of the protein quantity can be determined for example by an immunological detection of the protein. Moreover, biochemical techniques such as Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed. Depending on the type of the reduced protein product, its activity or the effect on the phenotype of the organism or the cell may also be determined.

“Protein quantity” refers to the amount of a particular polypeptide in an organism, a tissue, a cell or a cell compartment.

“Reduction” of the protein quantity refers to the quantitative reduction of the amount of a particular polypeptide in an organism, a tissue, a cell or a cell compartment—for example by the method according to the invention—in comparison with the wild type of the same genus and species to which this method has not been applied, under otherwise identical framework conditions (such as, for example, culture conditions, age, nutrient supply and the like). In this context, the reduction amounts to at least 10%, preferably at least 10% or at least 20%, especially preferably by at least 40% or 60%, very especially preferably by at least 70% or 80%, most preferably by at least 90% or 95%. Methods for determining the protein quantity are known to the skilled worker. Examples which may be mentioned are: the micro-Biuret method (Goa J (1953) Scand J Clin Lab Invest 5:218-222), the Folin-Ciocalteu method (Lowry O H et al. (1951) J Biol Chem 193:265-275) or measuring the absorption of CBB G-250 (Bradford M M (1976) Analyt Biochem 72:248-254).

“Different” in context with two endogenous target genes preferably means that the RNA or mRNA transcribed by the two endogenous target genes is not identical. Preferably, the homology of the RNA or mRNA transcribed by the two endogenous target genes is less than 90%, preferably less than 80%, especially preferably less than 70%, very especially preferably less than 60%, most preferably less than 50%, in each case over the entire length of the transcribed RNA or mRNA.

“At least partially double-stranded ribonucleic acid molecule” (hereinbelow dsRNA) means ribonucleic acid molecule which are entirely or partially double-stranded. Preferably, the ribonucleic acid sequence is predominantly completely double-stranded. “Predominantly completely double-stranded” means that at least 50%, preferably 70%, especially preferably 80%, very especially preferably 90%, of the bases in the molecule are present as a pair with another base of the dsRNA or can at least be theoretically present as a pair with another base, depending on the sequence of the dsRNA and the base pairing rules and, if appropriate, a prediction of the RNA secondary structure by means of a suitable computer algorithm.

“Essentially identical” means that a “sense” ribonucleotide sequence of the dsRNA may also have insertions, deletions and individual point mutations in comparison with the sequence of the “sense” RNA transcript of an endogenous target gene. Mutations comprise substitutions, additions, deletions, inversion or insertions of one or more bases of a nucleic acid sequence. Preferably, the homology between a “sense” ribonucleotide sequence of a dsRNA and at least part of the “sense” RNA transcript of an endogenous target gene amounts to at least 60%, preferably at least 70%, very especially preferably at least 90%, most preferably 95%. The sequences may also be identical with the corresponding sequence of the target gene. 100% sequence identity between the “sense” ribonucleotide sequence of the dsRNA and at least part of the “sense” strand of the transcript of an endogenous gene is preferred, albeit not necessarily required, for bringing about an efficient reduction of the expression of the endogenous gene. Individual mutations are tolerated. Accordingly, the method is tolerant to sequence deviations as may be present as the result of genetic mutations, polymorphisms or evolutionary divergences. Thus, for example, it is also possible to use a single dsRNA which has been generated starting from a particular endogenous gene, to suppress the expression of further homologous endogenous genes of the same organism or else the expression of homologous endogenous genes in other related species.

Homology is understood as meaning the extent of agreement between two nucleotide, ribonucleotide or protein sequences, which is preferably calculated by alignment with the aid of the program algorithm GAP (Wisconsin Package Version 10.0, University of Wisconsin, Genetics Computer Group (GCG), Madison, USA; Altschul et al. (1997) Nucleic Acids Res. 25:3389ff), setting the following parameters:

| | |
|---|---|
| Gap Weight: 50 | Length Weight: 3 |
| Average Match: 10 | Average Mismatch: 0 |

The skilled worker realizes that thymine (T) in the DNA sequence is regarded as the equivalent of uracil (U) in the RNA sequence when calculating the homology between DNA (for example genes) and RNA.

“Part of the “sense” RNA transcript of an endogenous target gene” means fragments of an RNA or mRNA transcribed by an endogenous target gene. In this context, said part preferably has a sequence length of at least 10 bases, preferably at least 25 bases, especially preferably at least 50 bases, very especially preferably at least 100 bases, most preferably at least 200 bases or at least 300 bases. Also comprised is the complete transcribed RNA or mRNA.

As an alternative, an “essentially identical” dsRNA can also be defined as a nucleic acid sequence which is capable of hybridizing with a part of a transcript, preferably of the mRNA, of an endogenous target gene (for example in 400 mM NaCl, 40 mM PIPES pH 6.4, 1 mM EDTA 50° C. or 70° C. for 12 to 16 hours or under different standard hybridization conditions).

“Standard hybridization conditions” is to be understood in the broad sense and refers to less stringent, but also—preferably—stringent hybridization conditions. Such hybridization conditions are described, inter alia, in Sambrook J, Fritsch E F, Maniatis T et al., in Molecular Cloning (A Laboratory Manual), 2nd edition, Cold Spring Harbor Laboratory Press, 1989, pages 9.31-9.57) or in Current Protocols in Molecular Biology, John Wiley & Sons, N.Y. (1989), 6.3.1-6.3.6.

For example, the conditions during the washing step can be selected from the range of conditions limited by those with low stringency (with approximately 2×SSC at 50° C.) and—preferably—those with high stringency (with approximately 0.2×SSC at 50° C., preferably at 65° C.) (20×SSC: 0.3 M sodium citrate, 3 M NaCl, pH 7.0). Moreover, the temperature during the washing step can be raised from low-stringency conditions at room temperature, approximately 22° C., up to—preferably—higher-stringency conditions at approximately 65° C. Both parameters, salt concentration and temperature, can be varied simultaneously, or else one of the two parameters can be kept constant while only the other is being varied. During the hybridization, denaturing agents such as, for example, formamide or SDS, may also be employed. In the presence of 50% formamide, the hybridization is preferably carried out at 42° C. Some examples of conditions for hybridization and washing step are detailed hereinbelow:
(1) Hybridization conditions be selected for example from the following conditions:
   a) 4×SSC at 65° C.,
   b) 6×SSC at 45° C.,
   c) 6×SSC, 100 μg/ml denatured fragmented fish sperm DNA at 68° C.,
   f) 50% formamide, 4×SSC at 42° C.,
   h) 2× or 4×SSC at 50° C. (low-stringency condition),
   i) 30 to 40% formamide, 2× or 4×SSC at 42° C. (low-stringency condition),
(2) washing steps can be selected for example from the following conditions:
   a) 0.015 M NaCl/0.0015 M sodium citrate/0.1% SDS at 50° C.
   b) 0.1×SSC at 65° C.
   c) 0.1×SSC, 0.5% SDS at 68° C.
   d) 0.1×SSC, 0.5% SDS, 50% formamide at 42° C.
   e) 0.2×SSC, 0.1% SDS at 42° C.
   f) 2×SSC at 65° C. (low-stringency condition).

“Essentially complementary” means that the “antisense” ribonucleotide sequences of the dsRNA may also have insertions, deletions and individual point mutations in comparison with the complement of the “sense” ribonucleotide sequences. Preferably, the homology is at least 80%, preferably at least 90%, very especially preferably at least 95%, most preferably 100%, between the “antisense” ribonucleotide sequences and the complement of the “sense” ribonucleotide sequences. Complement here means—in the manner with which the skilled worker is familiar—the counterstrand derived in accordance with the base pairing rules.

The double-stranded structure of the dsRNA can be formed starting from a single, fully or partially autocomplementary RNA strand (in which the abovementioned “sense” and “antisense” ribonucleotide sequences of the dsRNA are all linked covalently with one another) or starting from two RNA strands (in which the abovementioned “sense” and “antisense” ribonucleotide sequences of the dsRNA are located on separate strands) which are fully or partially complementary to one another. In the case of two separate strands, for example, all “sense” ribonucleotide sequences may be located on one strand, while all “antisense” ribonucleotide sequences are located on the other strand. However, the sequences can also be allocated in different ways to the two strands. The formation of the double-stranded structure can take place in-vitro, but also in-vivo, for example in the eukaryotic cell itself. Preferably, the dsRNA is present in the form of a single autocomplementary RNA strand.

The individual “sense” ribonucleotide sequences can form a double-stranded RNA structure with the corresponding, essentially complementary “antisense” ribonucleotide sequences by means of base pairing and form a subunit of the dsRNA.

In the case of an autocomplementary strand, there are various possibilities for the primary structures of the dsRNA. Those listed hereinbelow are to be understood as examples, but not by way of limitation:
a) It is possible first to add the “sense” ribonucleotide sequences (S) of the individual subunits to one another, which is followed by a sequential arrangement of the essentially complementary “antisense” ribonucleotide sequences (AS). The number of the units n is greater than or equal to two. This gives rise to a structure with a single hairpin. The primary structure of the dsRNA here can be for example as in the following scheme:
   5′-S(1)-S(2)-.....-S(n)-AS(n)-....-AS(2)-AS(1)-3′
   The preferred secondary structure is shown in FIG. 2-A.
b) It is possible first to add the “sense” ribonucleotide sequence (S) and the essentially complementary “antisense” ribonucleotide sequence (AS) of the first subunits to one another, which is followed by the sequential arrangement of “sense” and “antisense” ribonucleotide sequences of the further subunits. The number of the units n is greater than or equal to two. This gives rise to a structure with several hairpins. The primary structure of the dsRNA here can be for example as in the following scheme:
   5′-S(1)-AS(1)-S(2)-AS(2).....-S(n)-AS(n)-3′
   The preferred secondary structure is shown in FIG. 2-B.

If the dsRNA is—preferably—capable of forming a hairpin structure, the stem of the hairpin corresponds to the double-stranded portion of the dsRNA which is formed by base pairing between “sense” and “antisense” ribonucleotide sequence located on the same RNA molecule. Here, “sense” and “antisense” ribonucleotide sequences are preferably connected by a “linker”. The “linker” is preferably an intron, which can be spliced out of the dsRNA. Autocomplementary dsRNA structures starting from a single RNA molecule are preferred since they only require the expression of one construct and always comprise the complementary RNA strands in an equimolar ratio.

When using a linker (I)—preferably an intron—, the following schematic primary structures may be mentioned as examples of the dsRNA:
c) This is a preferred variant of a) in which a linker (I)—preferably an intron—is inserted at the position of the hairpin loop:
   5′-S(1)-S(2)-.....-S(n)-I-AS(n)-....-AS(2)-AS(1)-3′
   The preferred secondary structure is shown in FIG. 2-C.
d) This is a preferred variant of b), in which a linker (I)—preferably an intron—is inserted at the position of the each hairpin loop:
   5′-S(1)-I-AS(1)-S(2)-I-AS(2).....-S(n)-I-AS(n)-3′
   The preferred secondary structure is shown in FIG. 2-D.

However, the dsRNA molecules are also functional without the linker. In this case, however, it must be taken into consideration that the last approx. 10 nucleotides of the terminal subunit S(n) no longer undergo correct pairing. In this case, the length for this subunit is to be complemented by 10 nucleotides. The linker is preferably an intron, especially preferably an intron in sense orientation. It is preferably an intron of a plant gene. The following may be mentioned by way of example, but not by limitation: the intron 3 of the maize alcohol dehydrogenase 1 (Adh1) (GenBank Acc.-NO.: AF044293; GI: 2828164), the intron 4 of the soya beta-conglycinin alpha subunit (GenBank Acc.-NO.: AB051865); one of the introns of the pea rbcS-3A gene for the ribulose-1,5-bisphosphate carboxylase (RBC) small subunit (GenBank Acc.-NO.: X04333). The skilled worker is familiar with these and further suitable introns (McCullough A J & Schuler M A (1997) Nuc Acids Res 25:1071-1077). For the application in the method according to the invention, the intron is preferably employed in combination with splice acceptor and splice donor sequences, which make it possible that the dsRNA is spliced out at a later point in time. These splice sequences can be the flanking sequences of the intron themselves, or else be provided by corresponding sequences of the remainder of the dsRNA.

Each of the individual “sense” ribonucleotide sequences of the dsRNA is essentially identical to at least part of the “sense” RNA transcript of an endogenous target gene. However, not all “sense” ribonucleotide sequences in this context are identical to the “sense” RNA transcript of an individual endogenous target gene, but the maximum identity in each case, of at least two of the “sense” ribonucleotide sequences, is with the “sense” RNA transcripts of different endogenous target genes. In this case, the homology between the transcripts of the two endogenous target genes is under 90%, preferably under 80%, especially preferably under 70%, very especially preferably under 60%, most preferably under 50%.

At least two of the individual “sense” ribonucleotide sequences comprised in the dsRNA according to the invention are different. Different means firstly that the target genes with whose transcripts they have in each case the maximum identity are not identical. Preferably, at least one subunit of the dsRNA reduces the expression of another gene as at least one other subunit. Secondly, different can also mean that the “sense” ribonucleotide sequences of the subunits themselves are essentially not identical and preferably have under 60%, more preferably under 50%, even more preferably under 40% homology with one another. In a further embodiment, the dsRNA can comprise more than one copy of a subunit. Moreover, the dsRNA can also comprise a plurality of different subunits which, however, are directed against the same endogenous target gene and whose “sense” ribonucleotide sequences are, for example, essentially identical to different parts of the “sense” RNA transcript of said endogenous target gene.

In this context, each of the individual “sense” ribonucleotide sequences can also be essentially identical to the transcript of a plurality of endogenous target genes. This is the case in particular when the target genes have similar sequence segments, as is the case, for example, in members of gene families (for example storage proteins). This is an especially advantageous use form since—when a suitable ribonucleotide sequence of a subunit is chosen—said subunit can reduce the expression of more than one target gene.

Preferably, the sequence of the dsRNA is chosen in such a way that the desired dsRNA structure has the in each case least free energy after formation of the duplex in comparison with other possible folding variants of the primary structure of the dsRNA. This can be ensured for example by avoiding sequence duplications and the like. The specific secondary structure can be predicted and optimized for example using suitable computer programs (for example FOLDRNA; Zuker and Stiegler (1981) Nucleic Acids Res 9(1):133-48).

In a preferred embodiment, each subunit of the dsRNA has a length of at least 20 base pairs, preferably at least 50 base pairs, especially preferably at least 100 base pairs, very especially referably at least 250 base pairs.

In a furthermore preferred embodiment, each unit has a length of an integer multiple of 21 or 22 base pairs, that is to say, for example, 21, 22, 42, 43, 44, 63, 64, 65, 66, 84, 85, 86, 87, 88, 105, 106, 107, 108, 109, 110, 126, 127, 128, 129, 131, 132, 147, 148, 149, 150, 151, 152, 153, 154, 168, 169, 170, 171, 172, 173, 174, 175, 176, 189, 190, 191, 192, 193, 194, 195, 196, 197, 198, 210, 211, 212, 213, 214, 215, 216, 217, 218, 219 or 220 base pairs, preferably 21, 22, 42, 44, 63, 66, 84, 88, 105, 110, 126, 132, 147, 154, 168, 176, 189, 198, 210 or 220 base pairs, very especially preferably 21, 42, 63, 84, 105, 126, 147, 168, 189 or 210 base pairs, most preferably 180 or 210 base pairs.

The “sense” and/or “antisense” ribonucleotide sequences of the individual subunits can be linked with one another directly or else linked with one another and/or flanked by a spacer (SP). Individual spacers (SP) can be identical or else different. The spacer preferably meets the same requirements with regard to length as have been detailed above for the length of the subunits themselves. The spacer can form a double-stranded structure, but may also exist—for example in the form of a bubble—in unpaired formation, i.e. the bases in strand and counterstrand need not necessarily be complementary. Preferred embodiments are described for example by the following primary structures:
e) This is a preferred variant of c):
   5′SP—S(1)-SP—S(2)-SP-..-AS(n)-AS(n)-SP-..-AS(2)-SP-AS(1)-SP-3′
   The preferred secondary structure is shown in FIG. 3-A.

The spacer can comprise further functional elements. The following may be mentioned by way of example, but not by limitation:
i) Sequences encoding a recognition sequence (RE) which is recognized by a ribozyme as substrate. For example, the dsRNA can have the following linear structure prior to folding:
   5′-S(1)-(RE)-S(2)-...-S(n)-AS(n)-..-AS(2)-(RE)-AS(1)-3′
   The preferred secondary structure is shown in FIG. 3-B. The ribozyme (R) in question can be expressed separately, but can also be encoded on the dsRNA itself. Here, the sequence encoding a ribozyme is preferably arranged in such a way that, in the folded dsRNA molecule, it is opposite to a sequence which can act as substrate for this ribozyme. For example, the dsRNA can have the following linear structure prior to folding:
   5′-S(1)-(R)(RE)-S(2)-...-S(n)-AS(n)-..-AS(2)-(R)(RE)-AS(1)-3′
   The preferred secondary structure is shown in FIG. 3-C. Owing to the abovementioned use forms, the individual subunits are separated from one another after transcription, owing to the effect of the ribozyme. This separation is advantageous, but not necessarily required. Analogously utilizable ribozymes and recognition sequences are known to the skilled worker.
   Ribozymes refers to catalytic RNA molecules. Ribozymes can be adapted to any desired target. RNA and cleave the phosphodiester backbone at specific positions, whereby the target RNA is functionally deactivated (Tanner N K (1999) FEMS Microbiol Rev 23(3):257-275). The ribozyme itself is not modified thereby, but is capable of cleaving further target RNA molecules in an analogous manner, thus acquiring the properties of an enzyme. The incorporation of ribozyme sequences into “antisense” RNAs confers to precisely those “antisense” RNAs this enzyme-like, RNA-cleaving property and thus increases their efficiency in the inactivation of the target RNA. The preparation and use of suitable ribozyme “antisense” RNA molecules is described, for example, in Haseloff et al. (1988) Nature 334: 585-591. In this manner, ribozymes (for example “Hammerhead” ribozymes; Haselhoff and Gerlach (1988) Nature 334:585-591) can be used for catalytically cleaving the of a specific RNA. Methods for expressing ribozymes for reducing specific proteins have been described in (EP 0 291 533, EP 0 321 201, EP 0 360 257). Ribozyme expression in plant cells has likewise been described (Steinecke P et al. (1992) EMBO J. 11 (4):1525-1530; de Feyter R et al. (1996) Mol Gen Genet. 250(3):329-338). Suitable target sequences and ribozymes can be determined, for example as described in “Steinecke P, Ribozymes, Methods in Cell Biology 50, Galbraith et al. eds, Academic Press, Inc. (1995), pp. 449-460”, by secondary structure calculations of ribozyme RNA and target RNA, and by their interaction (Bayley C C et al. (1992) Plant Mol. Biol. 18(2):353-361; Lloyd A M and Davis R W et al. (1994) Mol Gen Genet. 242(6):653-657). For example, it is possible to construct derivatives of the tetrahymena L-19 IVS RNA which have regions which are complementary to the mRNA of the to the spacer sequences (see also U.S. Pat. No. 4,987,071 and U.S. Pat. No. 5,116,742). Alternatively, such ribozymes can also be identified from a library of various ribozymes by means of a selection process (Bartel D and Szostak J W (1993) Science 261:1411-1418).
ii) Sequences encoding recognition sequences for RNAses. The spacer can comprise recognition sequences for RNAses, preferably sequence-specific RNAses such as, for example, RNAse III. RNAse III cleaves at the motif 5′-AGNN-3 when four of these motifs are present in a loop (Nagel R & Ares M (2000) RNA 6:1142-1156). The RNAse can be a plant RNAse or else—as is the case for example in bacterial RNAse III proteins—expressed recombinantly.
iii) Sequences encoding intron splice signals (IS). Here, the splice donor and splice acceptor sequences are preferably located in such a way that in each case the subunit is spliced out in the form of an intron. Intron splice signals are described in Meritt et al. (1997) Plant Journal 12:937-943 or in Egoavil et al. (1997) Plant Journal 12:971-980.

The dsRNA or its precursor molecules can be introduced into an organism or a cell in various ways with which the skilled worker is familiar. “To introduce” is to be understood in the broad sense and comprises, for the purposes of the present invention, all those methods which are suitable for directly or indirectly introducing, into an organism or a cell, compartment, tissue, organ or seed of same, a dsRNA or its precursor molecules, or generating it/them therein. The introduction can bring about the transient presence of a dsRNA, or else a stable presence. Comprised are methods of the direct transfection or transformation of the cell with the as well as the transformation or transfection of the cell with expression cassettes which are capable of expressing, in the cell, the ribonucleic acid sequences on which the dsRNA is based (hereinbelow dsRNA expression system). The expression of the dsRNA can be transient or—for example after integration into the genome of the organism—stable. The duplex formation of the dsRNA can be initiated either outside or within the cell.

The dsRNA is introduced in an amount which makes possible the presence of at least one copy per cell. Higher amounts (for example at least 5, 10, 100, 500 or 1000 copies per cell) can, if appropriate, effect a more efficient reduction of the expression of the target genes. Since dsRNA is extraordinarily mobile within an organism, it is not necessarily required to apply the dsRNA into each cell of the organism. It suffices to introduce, or to express, the dsRNA into a or few cells, it then being possible for the activity according to the invention also to be achieved in other cells of the same organism.

A dsRNA—for example for use in a direct transformation or transfection—can be synthesized in vivo or in vitro by enzymatic, molecular-biological or chemico-synthetic methods. Eukaryotic, prokaryotic or bacteriophage RNA polymerases (such as, for example, T3, T7 or SP6 RNA polymerase) can be used for this purpose. Suitable methods for the in-vitro expression of RNA are described (WO 97/32016; U.S. Pat. No. 5,593,874; U.S. Pat. No. 5,698,425, U.S. Pat. No. 5,712,135, U.S. Pat. No. 5,789,214, U.S. Pat. No. 5,804,693). Prior to introduction into a cell, tissue or organism, a dsRNA which has been synthesized in vitro, either chemically or enzymatically, can be purified either completely or in part from the reaction mixture, for example by extraction, precipitation, electrophoresis, chromatography or combinations of these methods. The dsRNA can be introduced directly into the cell (for example by particle bombardment or microinjection) or else be applied extracellularly (for example into the interstitial space, the vascular system, the digestion system and the like). An application of, for example, dsRNA-expressing organisms in the form of food is also feasible. It is known that dsRNA has good cell penetration characteristics and sufficient stability. Owing to the high efficacy of the dsRNA, only few molecules suffice to obtain a good effect for the purposes of the invention.

Furthermore, modifications of both the sugar-phosphate backbone and of the nucleosides may be present in the dsRNA. For example, the phosphodiester bonds of the RNA can be modified in such a way that they comprise at least one nitrogen or sulfur hetero atom. Bases can be modified in such a way that the activity, for example of adenosine deaminase is restricted. The dsRNA can be generated enzymatically or, fully or in part, chemico-synthetically.

However, the dsRNA is preferably expressed in the cell starting from suitable expression systems. A further aspect of the invention relates to said dsRNA expression systems. If the dsRNA is expressed as a single, autocomplementary RNA strand, the expression system comprises an expression cassette with a DNA sequence encoding the autocbmplementary RNA strand and in operable linkage with a promoter which is suitable for ensuring the expression in the eukaryotic cell in question. The expression cassette can optionally comprise further functional elements such as, for example, transcription terminators and/or polyadenylation signals. Such expression cassettes are likewise an aspect of the invention.

If the dsRNA is expressed in the form of two separate strands which are fully or partially complementary to one another, the expression system comprises two expression cassettes where each of the two strands is linked operably with a promoter which is suitable for ensuring the expression in the eukaryotic cell in question. The expression cassettes can optionally comprise further functional elements such as, for example, transcription terminators and/or polyadenylation signals. The two expression cassettes can be combined to give the expression system according to the invention in various ways as known to the skilled worker. Examples which may be mentioned are:
a) transformation of the cell or plant with a vector comprising expression cassettes for both. RNA strands,
b) cotransformation of the cell or plant with two vectors, where in each case one vector encodes in each case one of the two strands of the dsRNA,
c) hybridization of two plants which have been transformed with in each case one vector, where in each case one vector encodes in each case one of the two strands of the dsRNA.

It is also possible to employ an expression cassette in which the dsRNA-encoding DNA sequence is located between two promoters with opposite direction of transcription, thus being transcribed from both sides.

Expression cassette refers to chimeric DNA molecules in which a nucleic acid sequence which encodes the dsRNA molecule (or one of the strands thereof) is linked to at least one genetic control element (for example a promoter, enhancer, silencer, splice donor, splice acceptor or polyadenylation signal) such that the transcription of the dsRNA molecule (or one of the strands thereof) in the eukaryotic cell or organism is ensured. Suitable advantageous constructions are described hereinbelow. Polyadenylation is possible, but not necessary, nor do elements for initiating a translation have to be present.

If the expression construct is to be introduced into a plant and the dsRNA is to be generated in plantae, plant-specific genetic control elements (for example plant-specific promoters) are preferred. However, the dsRNA can also be generated in other organisms or in vitro and then be introduced into the plant.

Operable linkage is understood as meaning, for example, the sequential arrangement of a promoter with the nucleic acid sequence to be transcribed and, if appropriate, further regulatory elements such as, for example, a terminator and/or polyadenylation signals in such a way that each of the regulatory elements can fulfill its function when the nucleic acid sequence is transcribed, depending on the arrangement of the nucleic acid sequences. To this end, a direct linkage in the chemical sense is not necessarily required. Genetic control sequences such as, for example, enhancer sequences, can also exert their function on the target sequence in positions which are further away, or indeed from other DNA molecules. Arrangements are preferred in which the nucleic acid sequence to be transcribed is positioned behind the sequence acting as promoter, so that both sequences are covalently linked to one another. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the dsRNA.

An operable linkage and an expression cassette can be realized by means of customary recombination and cloning techniques as are described, for example, in Maniatis T, Fritsch E F and Sambrook J (1989) Molecular Cloning: A Laboratory Manual, Cold Spring Harbor Laboratory, Cold Spring Harbor (NY), in Silhavy T J, Berman M L and Enquist L W (1984) Experiments with Gene Fusions, Cold Spring Harbor Laboratory, Cold Spring Harbor (NY), in Ausubel F M et al. (1987) Current Protocols in Molecular Biology, Greene Publishing Assoc. and Wiley Interscience and in Gelvin et al. (1990) In: Plant Molecular Biology Manual.

However, an expression cassette is also understood as meaning those constructions in which for example a nucleic acid sequence encoding a dsRNA is placed in such a way behind an endogenous promoter that the same effect occurs. Both approaches give expression cassettes for the purposes of the invention.

Promoters which can be used for the method according to the invention are, in principle, all natural promoters together with their regulation sequences, such as those mentioned above, as long as they ensure expression in the target organism. Moreover, synthetic promoters can also be used advantageously.

Further promoters which make possible the expression in further eukaryotes or in prokaryotes, such as, for example, E. coli bacteria, may be linked operably with the nucleic acid sequence to be expressed.

The nucleic acid sequences which are present in the expression cassettes or vectors according to the invention can be linked operably with further genetic control sequences, in addition to a promoter. The term genetic control sequence is to be understood in the broad sense and refers to all those sequences which have an effect on the materialization or the function of the expression cassette according to the invention. Genetic control sequences modify for example transcription in prokaryotic or eukaryotic organisms. Preferably, the expression cassettes according to the invention comprise a plant-specific promoter 5′-upstream of the respective nucleic acid sequence to be expressed recombinantly, and 3′-downstream a terminator sequence as additional genetic control sequence, and, if appropriate, further customary regulatory elements, in each case linked operably with the nucleic acid sequence to be expressed recombinantly.

Genetic control sequences furthermore also comprise the 5′-untranslated regions, introns or noncoding 3′-region of genes. It has been demonstrated that these may play an important role in the regulation of gene expression. Control sequences furthermore comprise polyadenylation signals and terminator sequences.

The expression cassette can advantageously comprise one or more of what are known as enhancer sequences in operable linkage with the promoter, which sequences make possible an increased recombinant expression of the nucleic acid sequence. Additional advantageous sequences such as further regulatory elements or terminators may also be inserted at the 3′-end of the nucleic acid sequence to be expressed recombinantly. One or more copies of the nucleic acid sequences to be expressed recombinantly may be present in the gene construct.

Control sequences are furthermore understood as being those sequences which make possible a homologous recombination or insertion into the genome of a host organism, or which permit the removal from the genome. Methods such as the cre/lox technology permit a tissue-specific, if appropriate inducible, removal of the expression cassette from the genome of the host organism (Sauer B (1998) Methods. 14(4):381-92). In this method, specific flanking sequences (lox sequences), which later allow removal by means of cre recombinase, are attached to the target gene.

Preferably, the expression cassette consisting of a linkage of promoter and nucleic acid sequence to be transcribed can be present integrated in a vector and can be introduced into the eukaryotic cell or organism by, for example, transformation, by one of the methods described hereinbelow. The subsequent expression can be transient or else—preferably—stable after insertion (for example using selection markers) of the expression cassettes into the genome. Preferably, the dsRNA expression system is integrated stably into the genome—for example the chromosomal DNA or the DNA of the organelles (for example the plastids, mitochondria and the like)—of a cell.

The introduction, into an organism or cells, tissues, organs, parts or seeds of same (preferably into plants or plant cells, tissues, organs, parts or seeds), of a transgenic expression cassette according to the invention can advantageously be carried out using vectors in which the transgenic expression cassettes are present. Examples of vectors can be plasmids, cosmids, phages, viruses or else agrobacteria. The transgenic expression cassettes can be inserted into the vector (preferably a plasmid vector) via a suitable restriction cleavage site. The resulting vector is first introduced into E. coli. Correctly transformed E. coli are selected, grown, and the recombinant vector is obtained by methods with which the skilled worker is familiar. Restriction analysis and sequencing can be employed for verifying the cloning step. Preferred vectors are those which make possible a stable integration of the expression cassette into the host genome.

The generation of a transformed organism (or of a transformed cell or tissue) requires introducing the relevant DNA (for example the expression vector) or RNA into the relevant host cell. A multiplicity of methods (Keown et al. (1990) Methods in Enzymology 185:527-537) is available for this method which is referred to as transformation (or transduction or transfection). Thus, for example, the DNA or RNA can be introduced directly by microinjection or by bombardment with DNA-coated microparticles. Also, the cell can be permeabilized chemically, for example using polyethylene glycol, so that the DNA can enter the cell by diffusion. The DNA can also be effected by protoplast fusion with other DNA-comprising units such as minicells, cells, lysosomes or liposomes. Electroporation is a further suitable method for introducing DNA, where the cells are permeabilized reversibly by an electrical impulse. Suitable methods are described (for example in Bilang et al. (1991) Gene 100:247-250; Scheid et al. (1991) Mol Gen Genet 228:104-112; Guerche et al. (1987) Plant Science 52:111-116; Neuhause et al. (1987) Theor Appl Genet 75:30-36; Klein et al. (1987) Nature 327:70-73; Howell et al. (1980) Science 208:1265; Horsch et al. (1985) Science 227:1229-1231; DeBlock et al. (1989) Plant Physiology 91:694-701; Methods for Plant Molecular Biology (Weissbach and Weissbach, eds.) Academic Press Inc. (1988); and Methods in Plant Molecular Biology (Schuler and Zielinski, eds.) Academic Press Inc. (1989)).

A further aspect of the invention relates to cells which comprise one of the dsRNA molecules, expression systems, expression cassettes or expression vectors according to the invention. The cell may be derived from an organism or be present in same, but also refers to single-celled organisms such as microorganisms. The cell can be prokaryotic or of eukaryotic nature. The method according to the invention is applied to eukaryotic organisms. However, prokaryotic organisms may still comprise the expression systems according to the invention, for example for the purposes of dsRNA production. Also, prokaryotic organisms, for example agrobacteria, can advantageously be employed as vehicles for the transformation of, for example, plant organisms.

Preferred prokaryotes are mainly bacteria such as bacteria of the genus Escherichia, Corynebacterium, Bacillus, Clostrridium, Proionibacterium, Butyrivibrio, Eubacterium, Lactobacillus, Erwinia, Agrobacterium, Flavobacterium, Alcaligenes, Phaeodactylum, Colpidium, Mortierella, Entomophthora, Mucor, Crypthecodinium or Cyanobacteria, for example of the genus Synechocystis. Microorganisms which are preferred are mainly those which are capable of infecting plants and thus of transferring the constructs according to the invention. Preferred microorganisms are those of the genus Agrobacterium and in particular the species Agrobacterium tumefaciens.

Eukaryotic cells and organisms comprises plant and animal, nonhuman organisms and/or cells and eukaryotic microorganisms such as, for example, yeasts, algae or fungi. A corresponding transgenic organism can be generated for example by introducing the expression systems in question into a zygote, stem cell, protoplast or another suitable cell which is derived from the organism.

“Animal organism” refers to nonhuman vertebrates or invertebrates. Preferred vertebrates comprise, for example, fish species, nonhuman mammals such as cattle, horse, sheep, goat, mouse, rat or pig, and birds such as chicken or goose. Preferred animal cells comprise CHO, COS, HEK293 cells. Invertebrates comprise nematodes or other worms, and insects. Invertebrates comprise insect cells such as Drosophila S2 and Spodoptera Sf9 or Sf21 cells.

Furthermore preferred are nematodes which are capable of attacking animals or humans such as those of the genera Ancylostoma, Ascaridia, Ascaris, Bunostomum, Caenorhabditis, Capillaria, Chabertia, Cooperia, Dictyocaulus, Haemonchus, Heterakis, Nematodirus, Oesophagostomum, Ostertagia, oxyuris, Parascaris, Strongylus, Toxascaris, Trichuris, Trichostrongylus, Tfhchonema, Toxocara or Uncinaria. Furthermore preferred are those which are capable of attacking plant organisms such as, for example, Bursaphalenchus, Criconemella, Diiylenchus, Ditylenchus, Globodera, Helicotylenchus, Heterodera, Longidorus, Melodoigyne, Nacobbus, Paratylenchus, Pratylenchus, Radopholus, Rotelynchus, Tylenchus or Xiphinema. Preferred insects comprise those of the genera Coleoptera, Diptera, Lepidoptera and Homoptera.

Preferred fungi are Aspergillus, Trichoderma, Ashbya, Neurospora, Fusarium, Beauveria or further fungi described in Indian Chem Engr. Section B. Vol 37, No 1,2 (1995), page 15, Table 6. Especially preferred is the filamentous Hemiascomycet Ashbya gossypii.

Preferred yeasts are Candida, Saccharomyces, Hansenula or Pichia, especially preferred are Saccharomyces cerevisiae or Pichia pastoris (ATCC Accession No. 201178).

Preferred as transgenic organisms are mainly plant organisms. “Plant organism” comprises any organism which is capable of photosynthesis, and the cells, tissues, parts or propagation material (such as seeds or fruits) derived therefrom. Encompassed within the scope of the invention are all genera and species of higher and lower plants of the Plant Kingdom. Annual, perennial, monocotyledonous and dicotyledonous plants and gymnosperms are preferred. Encompassed are mature plant, seed, shoots and seedlings, and parts, propagation material (for example tubers, seeds or fruits) and cultures, for example cell cultures or callus cultures, derived therefrom. Mature plants refer to plants at any developmental stage beyond the seedling stage. Seedling refers to a young, immature plant at an early developmental stage.

For the purposes of the invention, “plant” means all genera and species of higher and lower plants of the Plant Kingdom. This term includes the mature plants, seeds, shoots and seedlings, and parts, propagation material, plants organs, tissues, protoplasts, callus and other cultures, for example cell cultures, derived therefrom, and any other type of group of plant cells which give functional or structural units. Mature plants refer to plants at any developmental stage beyond the seedling stage. Seedling refers to a young, immature plant at an early developmental stage. “Plant” comprises all annual and perennial, monocotyledonous and dicotyledonous plants and includes by way of example, but not by limitation, those of the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solarium, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea and Populus.

Preferred are plants of the following plant families: Amaranthaceae, Asteraceae, Brassicaceae, Carophyllaceae, Chenopodiaceae, Compositae, Cruciferae, Cucurbitaceae, Labiatae, Leguminosae, Papilionoideae, Liliaceae, Linaceae, Malvaceae, Rosaceae, Rubiaceae, Saxifragaceae, Scrophulariaceae, Solanacea, Sterculiaceae, Tetragoniacea, Theaceae, Umbelliferae.

Preferred monocotyledonous plants are selected in particular from among the monocotyledonous crop plants such as, for example, the family of the Gramineae, such as alfalfa, rice, maize, wheat or other cereal species such as barley, millet and sorghum, rye, triticale or oats, and sugar cane, and also all grass species.

The invention is very especially preferably applied to dicotyledonous plant organisms. Preferred dicotyledonous plants are selected in particular from among the dicotyledonous crop plants such as, for example,
Asteraceae such as sunflower, tagetes or calendula and others,
Compositae, especially the genus Lactuca, very particularly the species sativa (lettuce) and others,
Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli) and other cabbages; and the genus Arabidopsis, very particularly the species thaliana, and cress or canola and others,
Cucurbitaceae such as melon, pumpkin/squash or zucchini and others,
Leguminosae, particularly the genus Glycine, very particularly the species max (soybean), soya, and alfalfa, pea, beans or peanut and others,
Rubiaceae, preferably the subclass Lamiidae such as, for example Coffea arabica or Coffea liberica (coffee bush) and others,
Solanaceae, particularly the genus Lycopersicon, very particularly the species esculentum (tomato) and the genus Solanum, very particularly the species tuberosum (potato) and melongena (aubergine) and tobacco or paprika and others,
Sterculiaceae, preferably the subclass Dilleniidae such as, for example, Theobroma cacao (cacao bush) and others,
Theaceae, preferably the subclass Dilleniidae such as, for example, Camellia sinensis or Thea sinensis (tea shrub) and others,
Umbelliferae, particularly the genus Daucus (very particularly the species carota (carrot)) and Apium (very particularly the species graveolens dulce (celery)) and others; and the genus Capsicum, very particularly the genus annum (pepper) and others,
   and linseed, soy, cotton, hemp, flax, cucumber, spinach, carrot, sugar beet and the various tree, nut and grapevine species, in particular banana and kiwi fruit.

Also encompassed are ornamental plants, useful or ornamental trees, flowers, cut flowers, shrubs or turf. Plants which may be mentioned by way of example but not by limitation are angiosperms, bryophytes such as, for example, Hepaticae (liverwort) and Musci (mosses); pteridophytes such as ferns, horsetail and clubmosses; gymnosperms such as conifers, cycades, ginkgo and Gnetalae, the families of Rosaceae such as rose, Ericaceae such as rhododendron and azalea, Euphorbiaceae such as poinsettias and croton, Caryophyllaceae such as pinks, Solanaceae such as petunias, Gesneriaceae such as African violet, Balsaminaceae such as touch-me-not, Orchidaceae such as orchids, Iridaceae such as gladioli, iris, freesia and crocus, Compositae such as marigold, Geraniaceae such as geranium, Liliaceae such as dracena, Moraceae such as ficus, Araceae such as cheeseplant and many others.

Furthermore, plant organisms for the purposes of the invention are further organisms capable of being photosynthetically active such as, for example, algae, cyanobacteria and mosses. Preferred algae are green algae such as, for example, algae from the genus Haematococcus, Phaedactylum tricornatum, Volvox or Dunaliella. Synechocystis is particularly preferred.

Most preferred are
a) Plants which are suitable for oil production such as, for example, oilseed rape, sunflower, sesame, safflower (Carthamus tinctorius), olive tree, soybean, maize, peanut, castor-oil plant, oil palm, wheat, cacao shrub, or various nut species such as, for example, walnut, coconut or almond. Especially preferred among these, in turn, are dicotyledonous plants, in particular oilseed rape, soybean and sunflower.
b) Plants which serve for the production of starch, such as, for example, maize, wheat or potato.
c) Plants which are used as foodstuffs and or feeding stuffs and/or useful plant and in which a resistance to pathogens would be advantageous such as, for example, barley, rye, rice, potato, cotton, flax, linseed.
d) Plants which can serve for the production of fine chemicals such as, for example, vitamins and/or carotenoids such as, for example, oilseed rape.

Depending on the host organism, the organisms used in the method are grown or cultured in a manner with which the skilled worker is familiar. As a rule, microorganisms are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium, and, if appropriate, vitamins, at temperatures of between 0° C. and 100° C., preferably between 10° C. to 60° C., while passing in oxygen. The pH of the liquid medium can be kept at a constant value, that is to say regulated during the culturing period, or else not. The culture can be batchwise, semibatchwise or continuous. Nutrients can be provided at the beginning of the fermentation or fed in semicontinuously or continuously.

The subsequent application of the method according to the invention may be mentioned by way of example, but not by limitation:

I. Plant Biotechnology

The method according to the invention is preferably employed for the purposes of plant biotechnology for generating plants with advantageous properties. Thus, the suitability of the plants or their seeds as foodstuff or feeding stuff can be improved, for example via a modification of the compositions and/or the content of metabolites, in particular proteins, oils, vitamins and/or starch. Also, growth rate, yield or resistance to biotic or abiotic stress factors can be increased. The subsequent applications in the field of plant biotechnology are particularly advantageous. The possible target genes stated are to be understood by way of example, but not by limitation:
1. Improved protection against abiotic stress factors (heat, chill, drought, increased moisture, environmental toxins, UV radiation). It is preferred to reduce the expression of genes which are involved in stress reactions.
2. Modification of the composition and/or the content of fatty acids, lipids or oils
   A modification of the fatty acid contents or the fatty acid composition, preferably in an oil crop such as oilseed rape or sunflower, can be achieved, for example, by reducing the gene expression of fatty acid biosynthesis genes, preferably those selected from the group consisting of genes encoding acetyl transacylases, acyl transport proteins (“acyl carrier protein”), desaturases such as stearyl desaturases or microsomal Δ12-desaturases, in particular Fad2-1 genes, malonyl transacylase, β-ketoacyl-ACP synthetases, 3-keto-ACP reductases, enoyl-ACP hydrases, thioesterases such as acyl-ACP thioesterases, enoyl-ACP reductases. Various further advantageous approaches for modifying the lipid composition are described (Shure M et al. (1983) Cell 35:225-233; Preiss et al. (1987) Tailoring Genes for Crop Improvement (Bruening et al., eds.), Plenum Press, S.133-152; Gupta et al. (1988) Plant Mol. Biol. 10:215-224; Olive et al. (1989) Plant Mol Biol 12:525-538; Bhattacharyya et al. (1990) Cell 60:155-122; Dunwell J M (2000) J Exp Botany 51Spec No:487-96; Brar D S et al. (1996) Biotech Genet Eng Rev 13:167-79; Kishore G M and Somerville C R (1993) Curr Opin Biotech 4(2):152-8). Preferred are, in particular, Fad2 genes (for example those described by Genbank Acc. No.: AF124360 (Brassica carinata), AF042841 (Brassica rapa), L26296 (Arabidopsis thaliana), A65102 (Corylus avellana)). Further advantageous genes and methods for modifying the lipid content are described, for example, in U.S. Pat. No. 5,530,192 and WO 94/18337. An elevated lipid content can also be achieved by reducing the starch content, for example as the result of the reduced expression of enzymes of the carbohydrate metabolism (for example ADP-glucose pyrophosphorylases).
3. Modification of the carbohydrate composition
   A modification of the carbohydrate composition can be achieved for example by reducing the gene expression of carbohydrate metabolism genes or of carbohydrate biosynthesis genes, for example genes of the biosynthesis of amylose, pectins, cellulose or cell-wall carbohydrates. A multiplicity of cellular processes (maturation, storability, starch composition or starch content and the like) can thereby be influenced in an advantageous manner. Target genes which may be mentioned by way of example, but not by limitation, are phosphorylases, starch synthetases, Q-enzymes, sucrose-6-phosphate synthetases, sucrose-6-phosphate phosphatases, ADP-glucose pyrophosphorylases, branching enzymes, debranching enzymes and various amylases. The corresponding genes are described (Dunwell J M (2000) J Exp Botany 51Spec No:487-96; Brar D S et al. (1996) Biotech Genet Eng Rev 13:167-79; Kishore G M and Somerville C R (1993) Curr Opin Biotech 4(2):152-8). Advantageous genes for influencing the carbohydrate metabolism—in particular starch biosynthesis—are described in WO 92/11375, WO 92/11376, U.S. Pat. No. 5,365,016 and WO 95/07355.
4. Modification of the color or pigmentation
   A modification of the color or pigmentation, preferably of ornamentals, can be achieved for example by reducing the gene expression of flavonoid biosynthesis genes such as, for example, the genes of chalcone synthases, chalcone isomerases, phenylalanine ammonia lyases, dehydrokaempferol(flavone) hydroxylases such as flavanone 3-hydroxylases or flavanone 2-hydroxylases, dihydroflavonol reductases, dihydroflavanol 2-hydroxylases, flavonoid 3′-hydroxylases, flavonoid 5′-hydroxylases, flavonoid glycosyltransferases (for example glucosyltransferases such as UDPG:flavonoid 3-O-glucosyltransferases, UDPG:flavonol 7-O-glucosyltransferases or rhamnosyltransferases), flavonoid methyltransferases (such as, for example, SAM:anthocyanidin-3-(p-coumaroyl)rutinoside-5-glucoside-3′,5′, —O-methyltransferases) and flavonoid acyltransferases (Hahlbrock (1981) Biochemistry of Plants, Vol. 7, Conn (Ed.); Weiring and de Vlaming (1984) “Petunia”, K C Sink (Ed.), Springer-Verlag, New York). Particularly suitable are the sequences described in EP-A1 522 880.
5. Reduction of the storage protein content
   The reduction of the gene expression of genes encoding storage proteins (SP hereinbelow) has a large number of advantages such as, for example, the reduction of the allergenic potential or modification in the composition or quantity of other metabolites. Storage proteins are described, inter alia, in EP-A 0 591 530, WO 87/47731, WO 98/26064, EP-A 0 620 281; Kohno-Murase J et al. (1994) Plant Mol Biol 26(4): 1115-1124.
   SP serve for the storage of carbon, nitrogen and sulfur, which are required for the rapid heterotrophic growth in the germination of seeds or pollen. In most cases, they have no enzymatic activity. SP are synthesized in the embryo only during seed development and, in this process, accumulate firstly in protein storage vacuoles (PSV) of differently differentiated cells in the embryo or endosperm.
   “Storage proteins” generally refers to a protein which has at least one of the subsequent essential properties:
   i) Storage proteins are essentially expressed only in the embryo during seed development. “Essentially” means that at least 50%, preferably at least 70%, very especially preferably at least 90%, most preferably at least 95%, of the total expression over the lifetime of a plant takes place in said stage.
   ii) Storage proteins are broken down again when the seed germinates. The breakdown during germination amounts to at least 20%, preferably at least 50%, very especially preferably at least 80%.
   iii) Storage proteins account for a substantial amount of the total protein content of the nongerminating seed. Preferably, the storage protein amounts to more than 5% by weight of the total protein in the nongerminating seed of the wild-type plant, especially preferably to at least 10% by weight, very especially preferably to at least 20% by weight, most preferably to at least 30% by weight.
   Preferably, storage proteins have 2 or all of the abovementioned essential properties i), ii) or iii).
   Storage proteins can be classified into subgroups, as the function of further characteristic properties, such as, for example, their sedimentation coefficient or the solubility in different solutions (water, saline, alcohol). The sedimentation coefficient can be determined by means of ultracentrifugation in the manner with which the skilled worker is familiar (for example as described in Correia J J (2000) Methods in Enzymology 321:81-100).
   In total, four large gene families for storage proteins can be assigned, owing to their sequences: 2S albumins (napin-like), 7S globulins (phaseolin-like), 11S/12S globulins (legumin/cruciferin-like) and the zein prolamins.
   2S albumins are found widely in seeds of dicots, including important commercial plant families such as Fabaceae (for example soybean), Brassicaceae (for example oilseed rape), Euphorbiaceae (for example castor-oil plant) or Asteraceae (for example sunflower). 2S albumins are compact globular proteins with conserved cysteine residues which frequently form heterodimers.
   7S globulins occur in trimeric form and comprise no cysteine residues. After their synthesis, they are cleaved into smaller fragments and glycosylated, as is the case with the 2S albumins. Despite differences in polypeptide size, the different 7S globulins are highly conserved and can probably be traced to a shared precursor protein, as is the case with the 2S albumins. Only small amounts of the 7S globulins are found in monocots. In dicots, they always amount to less than the 11S/12S globulins.
   11S/12S globulins constitute the main fraction of the storage proteins in dicots, in addition to the 2S albumins. The high degree of similarity of the different 11S globulins from different plant genera, in turn, allow the conclusion of a shared precursor protein in the course of evolution.
   The storage protein is preferably selected from the classes of the 2S albumins (napin-like), 7S globulins (phaseolin-like), 11S/12S globulins (legumin/cruciferin-like) or zein prolamins.
   Especially preferred 2S albumins comprise
   i) 2S albumins from Arabidopsis, very especially preferably the 2S albumins of SEQ ID NO: 2, 4, 6 or 8, most preferably the proteins encoded by the nucleic acids as shown in SEQ ID NO: 1, 3, 5 or 7,
   ii) 2S albumins from species of the genus Brassica such as, for example, Brassica napus, Brassica nigra, Brassica juncea, Brassica oleracea or Sinapis alba, very especially preferably the 2S albumins with the SEQ ID NO: 32, 34, 36, 38, 40, 46 or 48, most preferably the proteins encoded by the nucleic acids as shown in SEQ ID NO: 31, 33, 35, 37, 39, 45 or 47,
   iii)₂S albumins from soybean, very especially preferably the 2S albumins with the SEQ ID NO: 42 or 44, most preferably the proteins encoded by the nucleic acids as shown in SEQ ID NO: 41 or 43,
   iv) 2S albumins from sunflower (Helianthus annus), very especially preferably the 2S albumins with the SEQ ID NO: 50 or 52, most preferably the proteins encoded by the nucleic acids as shown in SEQ ID NO: 49 or 51,
   and the corresponding homologs and functional equivalents to i) or ii) or iii) or iv) from identical or other plant species, in particular oilseed rape, sunflower, linseed, sesame, safflower, olive tree, soybean or various nut species. Functional equivalents are preferably distinguished by characteristic properties, such as a 2S-sedimentation coefficient and/or by a solubility in water, in addition to the abovementioned essential properties.
   In a further preferred embodiment, functional equivalents of the 2S albumins have at least 60%, preferably at least 80%, very especially preferably at least 90%, most preferably at least 95% homology with one of the protein sequences with the SEQ ID NO: 2, 4, 6, 8, 32, 34, 36, 38, 40, 42, 44, 46, 48, 50 or 52, where the homology extends preferably over a length of at least 30 amino acids, preferably at least 50 amino acids, especially preferably over 100 amino acids, most preferably over the entire length of the proteins in question, and have the same essential properties of a storage protein and—preferably—the characteristic properties of a 2S-storage protein.
   Especially preferred 7S globulins comprise those from Arabidopsis or soybean, very especially preferably the proteins with the SEQ ID NO: 94 or 96, most preferably the proteins encoded by the nucleic acids as shown in SEQ ID NO: 93 or 95. Functional equivalents are preferably distinguished by the characteristic properties such as a 7S-sedimentation coefficient and/or a solubility in saline, in addition to the abovementioned essential properties. As further characteristic property, 7S globulins can comprise no cysteine residues.
   In a further preferred embodiment, functional equivalents of the 7S globulins have at least 60%, preferably at least 80%, very especially preferably at least 90%, most preferably at least 95% homology with one of the protein sequences with the SEQ ID NO: 94 or 96, where the homology extends preferably over a length of at least 30 amino acids, preferably at least 50 amino acids, especially preferably over 100 amino acids, most preferably over the entire length of the proteins in question, and have the same essential properties of a storage protein and—preferably—the characteristic properties of a 7S-storage protein.
   Especially preferred 11S/12S globulins comprise preferably 11S globulins from oilseed rape, soybean and Arabidopsis, in particular
   i) 11S globulins from oilseed rape with the SEQ ID NO: 10, 12, 14, 16 or 18, most preferably the proteins encoded by the nucleic acids as shown in SEQ ID NO: 9, 11, 13, 15 or 17,
   ii) the 11S globulins from soybean with the SEQ ID NO: 20, 22, 24, 26 or 28, most preferably the proteins encoded by the nucleic acids as shown in SEQ ID NO: 19, 21, 23, 25 or 27,
   iii) the 11S globulins from Arabidopsis thaliana with the SEQ ID NO: 60, 62, 64, 66, 68 or 70, most preferably the proteins encoded by the nucleic acids as shown in SEQ ID NO: 59, 61, 63, 65, 67 or 69, and the corresponding homologs and functional equivalents from other plant species, in particular oilseed rape, sunflower, linseed, sesame, safflower, olive tree, soybean or various nut species, such as, for example, the sunflower 11S storage protein (SEQ ID NO: 30), in particular the protein encoded by the nucleic acid sequence as shown in SEQ ID NO: 29. Functional equivalents are preferably distinguished by characteristic properties such as an 11S- or 12S-sedimentation coefficient and/or by a solubility in saline (PBS; phosphate-buffered saline) and/or poor solubility in water, in addition to the abovementioned essential properties.
   In a further preferred embodiment, functional equivalents of the 11S or 12S albumins have at least 60%, preferably at least 80%, very especially preferably at least 90%, most preferably at least 95% homology with one of the protein sequences with the SEQ ID NO: 10, 12, 14, 16, 18, 20, 22, 24, 26, 28, 30, 60, 62, 64, 66, 68 or 70, where the homology extends preferably over a length of at least 30 amino acids, preferably at least 50 amino acids, especially preferably over 100 amino acids, most preferably over the entire length of the proteins in question, and have the same essential properties of a storage protein and—preferably—the characteristic properties of an 11S or 12S-storage protein.
   Especially preferred zein prolamins preferably comprise those from monocotyledonous plants, in particular maize, rice, oats, barley or wheat. Very especially preferred are the maize zein prolamins described by SEQ ID NO: 98, 100, 102 or 104—in particular the protein encoded by SEQ ID NO 97, 99, 101 or 103—, the rice prolamin as shown in SEQ ID NO: 106—in particular the protein encoded by SEQ ID NO 105—, the oat prolamin as shown in SEQ ID NO: 108—in particular the proteins encoded by SEQ ID NO 107—, the barley prolamin as shown in SEQ ID NO: 110 and/or 111—in particular the protein encoded by SEQ ID NO 109—and the wheat prolamin as shown in SEQ ID NO: 113—in particular the protein encoded by SEQ ID NO 112. Functional equivalents are preferably distinguished by solubility in 70% ethanolic solution and poor solubility in water or salt solution.
   In a further preferred embodiment, functional equivalents of the zein prolamins have at least 60%, preferably at least 80%, very especially preferably at least 90%, most preferably at least 95% homology with one of the protein sequences with the SEQ ID NO: 98, 100, 102, 104, 106, 108, 110, 111 or 113, where the homology extends preferably over a length of at least 30 amino acids, preferably at least 50 amino acids, especially preferably over 100 amino acids, most preferably over the entire length of the proteins in question, and have the same essential properties of a storage protein and—preferably—the characteristic properties of a zein prolamin.
   Functional equivalents means in particular natural or artificial mutations of the abovementioned storage proteins and homologous polypeptides from other plants with the same essential and—preferably—characteristic properties. Preferred are homologous polypeptides from above-described preferred plants. The sequences from other plants—for example those whose genomic sequence is known fully or in part, such as, for example, from Arabidopsis thaliana, Brassica napus, Nicotiana tabacum or Solanum tuberosum—which are homologous to the storage proteins disclosed within the scope of the present invention can be found by homology alignments from databases, can be found readily for example by database search or screening genetic libraries using the storage protein sequences mentioned by way of example as search sequence or probe.
   Mutations comprise substitutions, additions, deletions, inversion or insertions of one or more amino acid residues.
   A further aspect of the invention comprises an at least partially double-stranded ribonucleic acid molecule, wherein the double-stranded ribonucleic acid molecule comprises
   i) a “sense” RNA strand comprising at least two ribonucleotide sequence segments, where in each case at least one of these ribonucleotide sequence segments is essentially identical to at least part of the “sense” RNA transcript of a storage protein nucleic acid sequence as shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 59, 61, 63, 65, 67, 69, 71, 93, 95, 97, 99, 101, 103, 105, 107, 109 or 112 or of a functional equivalent thereof, but where not all ribonucleotide sequence segments are identical to the “sense” RNA transcript of a single of a storage protein nucleic acid sequence, and
   ii) an “antisense” RNA strand which is essentially complementary to the RNA sense strand of i).
   Preferably, at least two of the storage protein nucleic acid sequences to whose “sense” RNA transcript said ribonucleotide sequence segments are essentially identical, have less than 90%, preferably less than 80%, very especially preferably less than 60%, most preferably less than 50% homology with one another over the entire length of their coding nucleotide sequence.
   In a further preferred embodiment, the dsRNA comprises a plurality of sequence segments which bring about a simultaneous suppression of a plurality of storage proteins, preferably of storage proteins from different classes, such as, for example, a 2S albumin, 7S globulins, 11S/12S globulin or the zein prolamins.
   Most preferred are double-stranded RNA molecules described by the ribonucleic acid sequence as shown in SEQ ID NO: 84, 86 or 88. These are preferably coded by nucleotide sequences corresponding to SEQ ID NO: 83, 85 or 87.
5. Obtaining a resistance to plant pathogens
   A resistance to plant pathogens such as arachnids, fungi, insects, nematodes, protozoans, viruses, bacteria and diseases can be achieved by reducing the gene expression of genes which are essential for the growth, survival, certain developmental stages (for example pupation) or the multiplication of a certain pathogen. A suitable reduction can bring about a complete inhibition of the above steps, but also a delay of same. This may be plant genes which, for example, allow the pathogen to enter, but may also be pathogen-homologous genes. Preferably, the dsRNA is directed against genes of the pathogen. In this context, the dsRNA itself, but also the expression systems, expression cassettes or transgenic organisms may act as antipathogenic agent. For example, plants can be treated with suitable formulations of abovementioned agents, for example sprayed or dusted, the plants themselves, however, may also comprise the agents in the form of a transgenic organism and pass them on to the pathogens, for example in the form of a stomach poison. Various essential genes of a variety of pathogens are known to the skilled worker (for example for nematode resistance: WO 93/10251, WO 94/17194).
   Most preferred as pathogen are fungal pathogens such as Phytophthora infestans, Fusarium nivale, Fusarium graminearum, Fusarium culmorum, Fusarium oxysporum, Blumeria graminis, Magnaporthe grisea, Scierotinia sclerotium, Septoria nodorum, Septoria tritici, Alternaria brassicae, Phoma lingam, bacterial pathogens such as Corynebacterium sepedonicum, Erwinia carotovora, Erwinia amylovora, Streptomyces scabies, Pseudomonas syringae pv. tabaci, Pseudomonas syringae pv. phaseolicola, Pseudomonas syringae pv. tomato, Xanthomonas campestris pv. malvacearum and Xanthomonas campestris pv. oryzae, and nematodes such as Globodera rostochiensis, G. pallida, Heterodera schachtii, Heterodera avenae, Ditylenchus dipsaci, Anguina tritici and Meloidogyne hapla.
   Resistance to viruses can be obtained for example by reducing the expression of a viral coat protein, a viral replicase, a viral protease and the like. A large number of plant viruses and suitable target genes are known to the skilled worker.
6. Prevention of stem break
   A reduced susceptibility to stem break can be obtained for example by reducing the gene expression of genes of the carbohydrate metabolism (see above). Advantageous genes are described (WO 97/13865, inter alia) and comprise tissue-specific polygalacturonases or cellulases.
7. Delay of fruit maturation
   Delayed fruit maturation can be achieved for example by reducing the gene expression of genes selected from the group consisting of polygalacturonases, pectin esterases, β-(1-4)glucanases (cellulases), β-galactanases (β-galactosidases), or genes of ethylene biosynthesis, such as 1-aminocyclopropane-1-carboxylate synthase, genes of carotenoid biosynthesis such as, for example, genes of prephytoene or phytoene biosynthesis, for example phytoene desaturases. Further advantageous genes are, for example, in WO 91/16440, WO 91/05865, WO 91/16426, WO 92/17596, WO 93/07275 or WO 92/04456.
8. Achieving male sterility. Suitable target genes are described in WO 94/29465, WO89/10396, WO 92/18625, inter alia.
9. Reduction of undesired or toxic plant constituents such as, for example, glucosinolates. Suitable target genes are described (in WO 97/16559, inter alia).
10. Delay of senescence symptoms. Suitable target genes are, inter alia, cinnamoyl-CoA:NADPH reductases or cinnamoyl alcohol dehydrogenases. Further target genes are described (in WO 95/07993, inter alia).
11. Modification of the lignification and/or the lignin content, mainly in tree species. Suitable target genes are described in WO 93/05159, WO 93/05160, inter alia.
12. Modification of the fiber content in foodstuffs, preferably in seeds, by reducing the expression of coffeic acid O-methyltransferase or of cinnamoyl alcohol dehydrogenase.
13. Modification of the fiber quality in cotton. Suitable target genes are described in U.S. Pat. No. 5,597,718, inter alia.
14. Reduction of the susceptibility to bruising of, for example, potatoes by reducing for example polyphenol oxidase (WO 94/03607) and the like.
15. Enhancement of vitamin E biosynthesis, for example by reducing the expression of genes from the homogentisate catabolic pathway such as, for example, homogentisate 1,2-dioxygenase (HGD; EC NO.: 1.13.11.5), maleyl-acetoacetate isomerase (MAAI; EC NO.: 5.2.1.2.) or fumaryl-acetoacetate hydrolase (FAAH; EC NO.: 3.7.1.2).
   A further aspect of the invention comprises an at least partially double-stranded ribonucleic acid molecule, wherein the double-stranded ribonucleic acid molecule-comprises
   i) a “sense” RNA strand comprising at least two ribonucleotide sequence segments, where in each case at least one of these ribonucleotide sequence segments is essentially identical to at least part of the “sense” RNA transcript of a gene from the homogentisate catabolic pathway as described in SEQ ID NO: 115, 116, 118 or 120 or of a functional equivalent thereof, but where not all ribonucleotide sequence segments are identical to the “sense” RNA transcript of a single of a storage protein nucleic acid sequence, and
   ii) an “antisense” RNA strand which is essentially complementary to the RNA sense strand of i).
16. Reduction of the nicotine content for example in tobacco by reduced expression of, for example, N-methyl-putrescin oxidase and putrescin N-methyltransferase.
17. Reduction of the caffein content in coffee bean (Coffea arabica) by reducing the gene expression of genes of caffein biosynthesis such as 7-methylxanthine 3-methyltransferase.
18. Reduction of the theophyllin content in tea (Camellia sinensis) by reducing the gene expression of genes of theophyllin biosynthesis such as, for example, 1-methylxanthine 3-methyltransferase.
19. Increase of the methionine content by reducing threonine biosynthesis, for example by reducing the expression of threonine synthase (Zeh M et al. (2001) Plant Physiol 127(3):792-802).

Further examples of advantageous genes are mentioned for example in Dunwell J M, Transgenic approaches to crop improvement, J Exp Bot. 2000;51 Spec No; pages 487-96.

Each of the abovementioned applications can be used as such on its own. Naturally, it is also possible to use more than one of the abovementioned approaches simultaneously. If, in this context, all approaches are used, the expression of at least two differing target genes as defined above is reduced. In this context, these target genes can originate from a single group of genes which is preferred for a use, or else be assigned to different use groups.

For using the methods according to the invention, the skilled worker has available well-known tools, such as expression vectors with promoters which are suitable for plants, and methods for the transformation and regeneration of plants. Plant-specific promoters means principally any promoter which is capable of governing the expression of genes, in particular foreign genes, in plants or plant parts, plant cells, plant tissues, plant cultures. In this context, the expression can be for example constitutive, inducible or development-specific. The following are preferred:
a) Constitutive promoters
   “Constitutive” promoters refers to those promoters which ensure expression in numerous, preferably all, tissues over a substantial period of plant development, preferably at all points in time of plant development (Benfey et al. (1989) EMBO J. 8:2195-2202). A promoter which is preferably used is, in particular, a plant promoter or a promoter which is derived from a plant virus. Especially preferred is the promoter of the CaMV cauliflower mosaic virus ³⁵S transcript (Franck et al. (1980) Cell 21:285-294; Odell et al. (1985) Nature 313:810-812; Shewmaker et al. (1985) Virology 140:281-288; Gardner et al. (1986) Plant Mol Biol 6:221-228) or the 19S CaMV Promoter (U.S. Pat. No. 5,352,605; WO 84/02913; Benfey et al. (1989) EMBO J. 8:2195-2202). A further suitable constitutive promoter is the “Rubisco small subunit (SSU)” promoter (U.S. Pat. No. 4,962,028), the legumin B promoter (GenBank Acc. No. X03677), the promoter of the Agrobacterium nopaline synthase, the TR dual promoter, the OCS (octopine synthase) promoter from Agrobacterium, the ubiquitin promoter (Holtorf S et al. (1995) Plant Mol Biol 29:637-649), the ubiquitin 1 promoter (Christensen et al. (1992) Plant Mol Biol 18:675-689; Bruce et al. (1989) Proc Natl Acad Sci USA 86:9692-9696), the Smas promoter, the cinnamyl alcohol dehydrogenase promoter (U.S. Pat. No. 5,683,439), the promoters of the vacuolar ATPase subunits or the promoter for a proline-rich protein from wheat (WO 91/13991), and further promoters of genes whose constitutive expression in plants is known to the skilled worker.
b) Tissue-specific promoters
   Furthermore preferred are promoters with specificities for the anthers, ovaries, flowers, leaves, stems, roots and seeds.
   Seed-specific promoters such as, for example, the promoter of phaseolin (U.S. Pat. No. 5,504,200; Bustos M M et al. (1989) Plant Cell 1(9):839-53), of the 2S albumin gene (Joseffson L G et al. (1987) J Biol Chem 262:12196-12201), of legumin (Shirsat A et al. (1989) Mol Gen Genet 215(2): 326-331), of the USP (unknown seed protein; Bäumlein H et al. (1991) Mol Gen Genet 225(3):459-67), of the napin gene (U.S. Pat. No. 5,608,152; Stalberg K et al. (1996) L Planta 199:515-519), of the sucrose binding protein (WO 00/26388) or the legumin B4 promoter (LeB4; Baumlein H et al. (1991) Mol Gen Genet 225: 121-128; Baeumlein et al. (1992) Plant Journal 2(2):233-9; Fiedler U et al. (1995) Biotechnology (NY) 13(10):1090f), the Arabidopsis oleosin promoter (WO 98/45461), the Brassica Bce4 promoter (WO 91/13980). Further suitable seed-specific promoters are those of the genes encoding the “high molecular weight glutenin” (HMWG), gliadin, branching enzyme, ADP-glucose pyrophosphatase (AGPase) or starch synthase.
   Furthermore preferred are promoters which permit a seed-specific expression in monocots such as maize, barley, wheat, rye, rice and the like. Promoters which can be employed advantageously are the promoter of the 1pt2 or 1pt1 gene (WO 95/15389, Wo 95/23230) or the promoters described in Wo 99/16890 (promoters of the hordein gene, the glutelin gene, the oryzin gene, the prolamin gene, the gliadin gene, the glutelin gene, the zein gene, the kasirin gene or the secalin gene). Further seed-specific promoters are described in WO89/03887.
   Tuber-, storage-root- or root-specific promoters such as, for example, the class I patatin promoter (B33), the promoter of the cathepsin D inhibitor from potato.
   Leaf-specific promoters such as promoter of the cytosolic FBPase from potato (WO 97/05900), the SSU promoter (small subunit) of Rubisco (ribulose-1,5-bisphosphate carboxylase) or the ST-LSI promoter from potato (Stockhaus et al. (1989) EMBO J. 8:2445-2451).
   Flower-specific promoters such as, for example, the phytoene synthase promoter (WO 92/16635) or the promoter of the P-rr gene (WO 98/22593).
   Anther-specific promoters such as the 5126 promoter (U.S. Pat. No. 5,689,049, U.S. Pat. No. 5,689,051), the glob-1 promoter and the y-zein promoter.
c) Chemically inducible promoters

The expression cassettes may also comprise a chemically inducible promoter (review: Gatz et al. (1997) Annu Rev Plant Physiol Plant Mol Biol 48:89-108) by means of which the expression of the exogenous gene in the plant can be controlled at a particular point in time. Such promoters such as, for example, the PRP1 promoter (Ward et al. (1993) Plant Mol Biol 22:361-366), salicylic-acid-inducible promoter (WO 95/19443), a benzenesulfonamide-inducible promoter (EP 0 388 186), a tetracyclin-inducible promoter (Gatz et al. (1992) Plant J 2:397-404), an abscisic-acid-inducible promoter (EP 0 335 528) or an ethanol- or cyclohexanone-inducible promoter (WO 93/21334) can likewise be used.
d) Stress- or pathogen-inducible promoters
   Further preferred promoters are those which are induced by biotic or abiotic stress such as, for example, the pathogen-inducible promoter of the PRP1 gene (Ward et al. (1993) Plant Mol Biol 22:361-366), the heat-inducible hsp70 or hsp80 promoter from tomato (U.S. Pat. No. 5,187,267), the chill-inducible alpha-amylase promoter from potato (WO 96/12814), the light-inducible PPDK promoter or the wounding-inducible pinII promoter (EP375091).
   Pathogen-inducible promoters comprise those of genes which are induced as the result of attack by pathogens such as, for example, genes of PR proteins, SAR proteins, β-1,3-glucanase, chitinase and the like (for example Redolfi et al. (1983) Neth J Plant Pathol 89:245-254; Uknes, et al. (1992) The Plant Cell 4:645-656; Van Loon (1985) Plant Mol Viral 4:111-116; Marineau et al. (1987) Plant Mol Biol 9:335-342; Matton et al. (1987) Molecular Plant-Microbe Interactions 2:325-342; Somssich et al. (1986) Proc Natl Acad Sci USA 83:2427-2430; Somssich et al. (1988) Mol Gen Genetics 2:93-98; Chen et al. (1996) Plant J 10:955-966; Zhang and Sing (1994) Proc Natl Acad Sci USA 91:2507-2511; Warner, et al. (1993) Plant J 3:191-201; Siebertz et al. (1989) Plant Cell 1:961-968(1989)).
   Also comprised are wounding-inducible promoters such as that of the pinII gene (Ryan (1990) Ann Rev Phytopath 28:425-449; Duan et al. (1996) Nat Biotech 14:494-498), of the wun1 and wun2 gene (U.S. Pat. No. 5,428,148), of the win1 and win2 gene (Stanford et al. (1989) Mol Gen Genet 215:200-208), of systemin (McGurl et al. (1992) Science 225:1570-1573), of the WIP1 gene (Rohmeier et al. (1993) Plant Mol Biol 22:783-792; Eckelkamp et al. (1993) FEBS Letters 323:73-76), of the MPI gene (Corderok et al. (1994) The Plant J 6(2):141-150) and the like.
e) Development-dependent promoters
   Further suitable promoters are, for example, fruit-maturation-specific promoters, such as, for example, the fruit-maturation-specific promoter from tomato (WO 94/21794, EP 409 625). Development-dependent promoters includes partly the tissue-specific promoters since individual tissues are, naturally, formed as a function of the development.

Especially preferred promoters are constitutive and seed-specific promoters.

Genetic control sequences also encompass further promoters, promoter elements or minimal promoters, all of which are capable of modifying the expression-governing characteristics. Thus, for example, genetic control sequences can bring about the tissue-specific expression additionally as a function of certain stress factors. Suitable elements have been described, for example, for water stress, abscisic acid (Lam E and Chua N H, J Biol Chem 1991; 266(26): 17131-17135) and heat stress (Schoffl F et al., Molecular & General Genetics 217(2-3):246-53, 1989).

Genetic control sequences furthermore also comprise the 5′-untranslated regions, introns or noncoding 3′ region of genes, such as, for example, the actin-1 intron, or the Adh1-S introns 1, 2 and 6 (general reference: The Maize Handbook, Chapter 116, Freeling and Walbot, Eds., Springer, N.Y. (1994)). It has been demonstrated that they can play a significant role in the regulation of gene expression. Thus, it has been demonstrated that 5′-untranslated sequences can enhance the transient expression of heterologous genes. An example which may be mentioned of such translation enhancers is the tobacco mosaic virus 5′ leader sequence (Gallie et al. (−1987) Nucl Acids Res 15:8693-8711) and the like. They can furthermore promote tissue specificity (Rouster J et al. (1998) Plant J 15:435-440).

Polyadenylation signals which are suitable as control sequences are plant polyadenylation signals, preferably those which correspond essentially to T-DNA polyadenylation signals from Agrobacterium tumefaciens, in particular of gene 3 of the T-DNA (octopin synthase) of the Ti plasmid pTiACHS (Gielen et al. (1984) EMBO J. 3:83-5 ff) or functional equivalents thereof. Examples of especially suitable terminator sequences are the OCS (octopin synthase) terminator and the NOS (nopalin synthase) terminator.

An expression cassettes and the vectors derived therefrom may comprise further functional elements. The term functional element is to be understood in the broad sense and means all those elements which have an effect on the generation, multiplication or function of the expression cassettes, vectors or transgenic organisms according to the invention. The following may be mentioned by way of example, but not by limitation:
a) Selection markers which confer a resistance to a metabolism inhibitor such as 2-deoxyglucose-6-phosphate (WO 98/45456), antibiotics or biocides, preferably herbicides, such as, for example, kanamycin, G 418, bleomycin, hygromycin or phosphinothricin etc. Especially preferred selection markers are those which confer resistance to herbicides. Examples which may be mentioned are: DNA sequences which encode phosphinothricin acetyltransferases (PAT) and inactivate glutamine synthase inhibitors (bar and pat gene), 5-enolpyruvylshikimate-3-phosphate synthase genes (EPSP synthase genes), which confer resistance to Glyphosat® (N-(phosphonomethyl)glycin), the gox gene (glyphosate oxidoreductase) encoding the enzyme degrading Glyphosat®, the deh gene (encoding a dehalogenase which inactivates dalapon), sulfonylurea- and imidazolinone-inactivating acetolactate synthases, and bxn genes, which encode bromoxynil-degrading nitrilase enzymes, the aasa gene, which confers resistance to the antibiotic apectinomycin, the streptomycin phosphotransferase (SPT) gene, which imparts resistance to streptomycin, the neomycin phosphotransferase (NPTII) gene, which confers resistance to kanamycin or geneticidin, the hygromycin phosphotransferase (HPT) gene, which confers resistance to hygromycin, the acetolactate synthase gene (ALS), which confers resistance to sulfonylurea herbicides (for example mutated ALS variants with, for example, the S4 and/or Hra mutation).
b) Reporter genes which encode readily quantifiable proteins and, via their color or enzyme activity, make possible an assessment of the transformation efficacy, or the site of expression or the timing of expression. Very especially preferred in this context are reporter proteins (Schenborn E, Groskreutz D. Mol Biotechnol. 1999; 13(1):29-44) such as the “green fluorescence protein” (GFP) (Sheen et al. (1995) Plant Journal 8(5):777-784), chloramphenicol transferase, a luciferase (Ow et al. (1986) Science 234:856-859), the aequorin gene (Prasher et al. (1985) Biochem Biophys Res Commun 126(3):1259-1268), β-galactosidase, very especially preferred is β-glucuronidase (Jefferson et al. (1987) EMBO J. 6:3901-3907).)
c) Origins of replication, which ensure a multiplication of the expression cassettes or vectors according to the invention in, for example, E. coli. Examples which may be mentioned are ORI (origin of DNA replication), the pBR322 ori or the P15A ori (Sambrook et al.: Molecular Cloning. A Laboratory Manual, 2nd ed. Cold Spring Harbor Laboratory Press, Cold Spring Harbor, N.Y., 1989).
d) Elements which are required for Agrobacterium-mediated plant transformation, such as, for example, the right or left border of the T-DNA, or the vir region.

To select cells which have successfully undergone homologous recombination, or else transformed cells, it is usually required additionally to introduce a selectable marker which confers, to the cells which have successfully undergone recombination, a resistance to a biocide (for example a herbicide), a metabolism inhibitor such as 2-deoxyglucose-6-phosphate (WO 98/45456) or an antibiotic. The selection marker permits the transformed cells to be selected from untransformed cells (McCormick et al. (1986) Plant Cell Reports 5:81-84).

A variety of methods and vectors for introducing genes into the genome of plants and for the regeneration of plants from plant tissues or plant cells are known (Plant Molecular Biology and Biotechnology (CRC Press, Boca Raton, Fla.), chapter 6/7, pp. 71-119 (1993); White FF (1993) Vectors for Gene Transfer in Higher Plants; in: Transgenic Plants, vol. 1, Engineering and Utilization, Ed.: Kung and Wu R, Academic Press, 15-38; Jenes B et al. (1993) Techniques for Gene Transfer, in: Transgenic Plants, vol. 1, Engineering and Utilization, Ed.: Kung and R. Wu, Academic Press, pp. 128-143; Potrykus (1991) Annu Rev Plant Physiol Plant Molec Biol 42:205-225; Halford N G, Shewry P R (2000) Br Med Bull 56(1):62-73). These include for example those mentioned above. In the case of plants, the described methods for the transformation and regeneration of plants from plant tissues or plant cells are utilized for transient or stable transformation. Suitable methods are mainly the transformation of protoplasts by polyethylene glycol induced DNA uptake, the liposome-mediated transformation (such as, for example, described in U.S. Pat. No. 4,536,475), biolistic methods using the gene gun (“particle bombardment” method; Fromm M E et al. (1990) Bio/Technology. 8(9):833-9; Gordon-Kamm et al. (1990) The Plant Cell 2:603), electroporation, the incubation of dry embryos in DNA-comprising solution, and microinjection. In the case of these “direct” transformation methods, the plasmid used need not meet any particular requirements. Simple plasmids, such as those of the pUC series, pBR322, M13 mp series, pACYC184 and the like can be used. If intact plants are to be regenerated from the transformed cells, an additional selectable marker gene must be located on the plasmid.

In addition to these “direct” transformation techniques, transformation can also be carried out by bacterial infection by eans of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611).

The strains Agrobacterium tumefaciens or Agrobacterium rhizogenes, which are in most cases used for the transformation of Agrombacterium, also one by bacterial infection by means of, comprise a plasmid (Ti or Ri plasmid) which is transferred to the plant following infection with Agrobaterium. Part of this plasmid, referred to as T-DNA (transferred DNA), is integrated into the genome of the plant cell. Alternatively, Agrobacterium can also be used to transfer, to plants, binary vectors (mini Ti plasmids) and to integrate them into the plant genome. The Agrobacterium-mediated transformation is best suited to dicotyledonous diploid plant cells, while the direct transformation techniques are suitable for any cell type. Methods for the Agrobacterium-mediated transformation are described, for example, in Horsch RB et al. (1985) Science 225:1229f. If Agrobacteria are used, the expression cassette is to be integrated into specific plasmids, either into a shuttle, or intermediate, vector or into a binary vector. If a Ti or Ri plasmid is to be used for the transformation, at least the right border, but in most cases the right and the left border, of the Ti or Ri plasmid T-DNA is linked flanking region with the expression cassette to be introduced.

It is preferred to use binary vectors for the Agrobacterium tranformation. Binary vectors are capable of replicating both in E. coli and in Agrobacterium. As a rule, they comprise a selection marker gene and a linker or polylinker flanked by the right and left T-DNA border sequence. They can be transformed directly into Agrobacterium (Holsters et al. (1978) Mol Gen Genet 163:181-187). The selection marker gene permits a selection of transformed Agrobacteria and is, for example, the nptII gene, which confers resistance to kanamycin. The Agrobacterium which acts as host organism in this case should already comprise a plasmid with the vir region. This region is required for transferring the T-DNA to the plant cell. An Agrobacterium transformed thus can be used for the transformation of plant cells. The use of T-DNA for the transformation of plant cells has been studied and described extensively (EP 120 516; Hoekema, In: The Binary Plant Vector System, Offsetdrukkerij Kanters B. V., Alblasserdam, Chapter V; An et al. (1985) EMBO J. 4:277-287). A variety of binary vectors are known and, in some cases, commercially available, such as, for example, pBI101.2 or pBIN19 (Clontech Laboratories, Inc. USA; Bevan et al. (1984) Nucl Acids Res 12:8711), pBinAR, pPZP200 or PPTV.

The Agrobacteria transformed with such a vector can then be used in the known manner for the transformation of plants, in particular crop plants such as, for example, oilseed rape, for example by bathing scarified leaves or leaf segments in an Agrobacterial solution and subsequently growing them in suitable media. The transformation of plants by Agrobacteria is described (white FF, Vectors for Gene Transfer in Higher Plants; in Transgenic Plants, Vol. 1, Engineering and Utilization, edited by S. D. Kung and R. Wu, Academic Press, 1993, pp. 15-38; Jenes B et al. (1993) Techniques for Gene Transfer, in: Transgenic Plants, Vol. 1, Engineering and Utilization, edited by S. D. Kung and R. Wu, Academic Press, pp. 128-143; Potrykus (1991) Annu Rev Plant-Physiol Plant Molec Biol 42:205-225). Transgenic plants can be regenerated in the known manner from the transformed cells of the scarified leaves or leaf segments, and these transgenic plants comprise the above-described expression systems according to the invention integrated into them.

Stably transformed cells, i.e. those which comprise the introduced DNA integrated into the DNA of the host cell can be selected from untransformed cells when a selectable marker is component of the introduced DNA. A marker can be for example any gene which is capable of conferring a resistance to antibiotics or herbicides (such as kanamycin, G418, bleomycin, hygromycin or phosphinothricin and the like); (see above). Transformed cells which express such a marker gene are capable of surviving in the presence of concentrations of a relevant antibiotic or herbicide which destroy an untransformed wild type. Example are mentioned above and comprise preferably the bar gene which confers resistance to the herbicide phosphinothricin (Rathore K S et al. (1993) Plant Mol Biol 21(5):871-884), the nptII gene, which confers resistance to kanamycin, the hpt gene, which confers resistance to hygromycin, or the EPSP gene, which confers resistance to the herbicide glyphosate. The selection marker permits the selection of transformed cells from untransformed cells (McCormick et al. (1986) Plant Cell Reports 5:81-84). The plants obtained can be grown and hybridized in the customary manner. Preferably, two or more generations should be cultured to ensure that the genomic integration is stable and hereditary.

As soon as a transformed plant cell has been generated, an intact plant can be obtained using methods with which the skilled worker is familiar. Here, the starting material is, for example, callus cultures. Shoot and root development in these as yet undifferentiated cell masses can be induced in the known manner. The resulting plantlets can be planted and grown. Suitable methods are described (Fennell et al. (1992) Plant Cell Rep. 11: 567-570; Stoeger et al (1995) Plant Cell Rep. 14:273-278; Jahne et al. (1994) Theor Appl Genet 89:525-533).

The expression efficacy of the recombinantly expressed nucleic acids can be determined for example in vitro by shoot-meristem propagation using one of the above-described selection methods. Moreover, changes in the nature and level of the expression of a target gene and the effect on the phontype of the plant can be tested in greenhouse experiments using test plants.
II. Medicinal applications
   The dsRNA, expression systems or organisms provided in accordance with the invention are suitable for the preparation of pharmaceuticals for the treatment of human and animal diseases. For an efficient therapy, it is frequently insufficient to reduce only a single target gene. The method according to the invention is particularly suitable for the treatment of
      infection with pathogens, such as, for example, viral or bacterial diseases. In these cases, approaches which are directed against just one molecular target frequently lead to the development of resistances. However, a combination therapy, which deals with a plurality of targets, is complicated to coordinate and, above all, can only be evaluated with great difficulty in clinical experiments. Here, the method according to the invention makes possible an advantageous alternative. The inhibitory dsRNA can be applied in a manner with which the skilled worker is familiar. dsRNA is amazingly stable and efficient and can be applied for example by feeding suitable dsRNA-expressing bacteria. The method is particularly suitable for the treatment of viral infections, for example infections with the human immunodeficiency virus (HIV), by simultaneously reducing the expression of at least two viral genes, for example in the case of HIV genes such as gp41, which are responsible for cell penetration, and the viral replicase or reverse transcriptase.
      treatment of cancer (for example solid tumors and/or leukemias). Here, the skilled worker is familiar with a large number of potential target genes (for example oncogenes such as ABL1, BCL1, BCL2, BCL6, CBFA2, CBL, CSF1R, ERBA, ERBB, EBRB2, FGR, FOS, FYN, HRAS, JUN, LCK, LYN, MYB, MYC, NRAS, RET or SRC; tumor suppressor genes such as BRCA1 or BRCA2; adhesion molecules; cyclin kinases and their inhibitors).
   Further diseases which can potentially be treated with the method according to the invention, and the corresponding target genes, are available to the skilled worker without problems and comprise, for example, diseases of the cardiovascular system such as hypertension, diseases of the central or peripheral nervous system such as Alzheimer's disease, Parkinson's disease or multiple sclerosis and the like. Also, the method according to the invention makes possible the parallel treatment of more than one disease, such as, for example, a cardiovascular disease and a disease of the central nervous system, which is not possible when traditional approaches are used. Such approaches are advantageous especially in the case of multiple diseases as occur frequently with advanced age. An example which may be mentioned is the parallel treatment of hypertension and, for example, Alzheimer's disease or senile dementia. Here, these applications as such can be used in isolation. Naturally, it is also possible to use more than one of the abovementioned approaches simultaneously. In all applications, the expression of at least two different target genes is reduced. These target genes can be from the group of genes which is preferred for an application, or else belong to different application groups.
III. Biotechnological applications
   The method according to the invention can be applied advantageously in biotechnological methods. An application which may be mentioned by way of example but not by limitation, is the optimization of metabolic pathways in yeasts, fungi or other eukaryotic microorganisms or cells which are used in fermentation for the production of fine chemicals such as amino acids (for example lysin or methionin), vitamins (such as vitamin B2, vitamin C, vitamin E), carotenoids, oils and fats, polyunsaturated fatty acids, biotin and the like. In this context, of these applications can be applied as such in isolation. Naturally, it is also possible to use more than one of the abovementioned approaches simultaneously. In all applications, the expression of at least two different target genes is reduced. These target genes can be from the group of genes which is preferred for an application, or else belong to different application groups.

Vectors for expression in E. coli are preferably pQE70, pQE60 and pQE-9 (QIAGEN, Inc.); pBluescript vectors, Phagescript vectors, pNH8A, pNH16a, pNH18A, pNH46A (Stratagene Cloning Systems, Inc.); ptrc99a, pKK223-3, pKK233-3, pDR540, pRIT5 (Pharmacia Biotech, Inc.).

Preferred vectors for expression in eukaryotes comprise pWLNEO, pSV2CAT, pOG44, pXT1 and pSG (Stratagene Inc.); pSVK3, pBPV, pMSG and pSVL (Pharmacia Biotech, Inc.). Inducible vectors which may be mentioned are pTet-tTak, pTet-Splice, pcDNA4/TO, pcDNA4/TO/LacZ, pcDNA6/TR, pcDNA4/TO/Myc-His/LacZ, pcDNA4/TO/Myc-His A, pcDNA4/TO/Myc-His B, pcDNA4/TO/Myc-His C, pVgRXR (Invitrogen, Inc.) or the pMAM series (Clontech, Inc.; GenBank Accession No.: U02443). These vectors already provide the inducible regulatory control element, for example for a chemically inducible expression of a DSBI enzyme. The nucleic acid sequence encoding a DSBI enzyme can be inserted directly into these vectors.

Vectors for expression in yeast comprise for example pYES2, PYD1, pTEF1/Zeo, pYES2/GS, pPICZ, pGAPZ, pGAPZalph, pPIC9, pPIC3.5, PHIL-D2, PHIL-S1, pPIC3SK, pPIC9K and PA0815 (Invitrogen, Inc.).

Advantageous control sequences are for example the Gram-positive promoters amy and SPO2 and the yeast or fungal promoters ADC1, MFa, AC, P-60, CYC1, GAPDH, TEF, rp28, ADH.

Cloning vectors and techniques for the genetic manipulation of ciliates and algae are known to the skilled worker (WO 98/01572; Falciatore et al. (1999) Marine Biotechnology 1(3):239-251; Dunahay et al. (1995) J Phycol 31:10004-1012).

Selection markers which can be used are, in principle, many of the selection systems which are also preferred for plants. Especially preferred are for mammalian cell the neomycin (G418) resistance, the hygromycin resistance, the zeocin resistance or the puromycin resistance. The ampicillin resistance, the kanamycin resistance or the tetracyclin resistant are especially preferred for prokaryotes.

In principle, for the transformation of animal cell or of yeast cells, similar methods as the “direct” tranformation of plant cells are to be applied. In particular, methods such as the calcium-phosphate- or liposome-mediated transformation or else electroporation are preferred.

A further aspect of the invention relates to the use of the transgenic organisms according to the invention and of the cells, cell cultures, parts—such as, for example, in the case of transgenic plant organisms roots, leaves and the like—derived from them and transgenic propagation material such as seeds or fruits for the production of foodstuffs or feeding stuffs, pharmaceuticals or fine chemicals, such as, for example, enzymes, vitamins, amino acids, sugars, fatty acids, natural or synthetic flavorings, aromas and colorants. Especially preferred is the production of triacylglycerides, lipids, oils, fatty acids, starches, tocopherols and tocotrienols and carotenoids. Genetically modified plants according to the invention which can be consumed by humans and animals can also be used as foodstuffs or feeding stuffs, for example directly or after undergoing a processing which is known per se.

Sequences

1. SEQ ID NO: 1
   Nucleic acid sequence encoding A. thaliana albumin 2S subunit 1 (GenBank Acc. No.: M22032)
2. SEQ ID NO: 2
   Protein sequence encoding A. thaliana albumin 2S subunit 1
3. SEQ ID NO: 3
   Nucleic acid sequence encoding A. thaliana albumin 2S subunit 3 (GenBank Acc. No.: M22035)
4. SEQ ID NO: 4
   Protein sequence encoding A. thaliana albumin 2S subunit 3
5. SEQ ID NO: 5
   Nucleic acid sequence encoding A. thaliana albumin 2S subunit 2 (GenBank Acc. No.: M22034)

6. SEQ ID NO: 6
Protein sequence encoding A. thaliana albumin 2S subunit 2
7. SEQ ID NO: 7
   Nucleic acid sequence encoding A. thaliana albumin 2S subunit 4 (GenBank Acc. No.: M22033)
8. SEQ ID NO: 8
   Protein sequence encoding A. thaliana albumin 2S subunit 4
9. SEQ ID NO: 9
   Nucleic acid sequence encoding B. napus cruciferin storage protein (GenBank Acc. No.: X59294)
10. SEQ ID NO: 10
   Protein sequence encoding B. napus cruciferin storage protein
11. SEQ ID NO: 11
   Nucleic acid sequence encoding Brassica napus cruciferin (GenBank Acc. No.: X14555)
12. SEQ ID NO: 12
   Protein sequence encoding Brassica napus cruciferin
13. SEQ ID NO: 13
   Nucleic acid sequence encoding B. napus BnC2 cruciferin storage protein (GenBank Acc. No.: X59295)
14. SEQ ID NO: 14
   Protein sequence encoding B. napus BnC2 cruciferin storage protein
15. SEQ ID NO: 15
   Partial nucleic acid sequence encoding B. napus cruciferin cru4 subunit (GenBank Acc. No.: X57848)
16. SEQ ID NO: 16
   Partial protein sequence encoding B. napus cruciferin cru4 subunit
17. SEQ ID NO: 17
   Nucleic acid sequence encoding B. napus crul cruciferin subunit (GenBank Acc. No.: X62120)
18. SEQ ID NO: 18
   Protein sequence encoding B. napus crul cruciferin subunit
19. SEQ ID NO: 19
   Nucleic acid sequence encoding glycinin A-1a-B-x subunit from soybean (GenBank Acc. No.: M36686)
20. SEQ ID NO: 20
   Protein sequence encoding glycinin A-1a-B-x subunit from soybean
21. SEQ ID NO: 21
   Nucleic acid sequence encoding soybean glycinin subunit G2 (GenBank Acc. No.: X15122)
22. SEQ ID NO: 22
   Protein sequence encoding soybean glycinin subunit G2
23. SEQ ID NO: 23
   Nucleic acid sequence encoding soybean A5A4B3 glycinin subunits (GenBank Acc. No.: X02626)
4. SEQ ID NO: 24
   Protein sequence encoding soybean A5A4B3 glycinin subunits
25. SEQ ID NO: 25
   Nucleic acid sequence encoding soybean (G. max) glycinin storage protein subunit A3-B4 (GenBank Acc. No.: M10962)
26. SEQ ID NO: 26
   Protein sequence encoding soybean (G. max) glycinin storage protein subunit A3-B4
27. SEQ ID NO: 27
   Nucleic acid sequence encoding soybean glycinin subunit G3 (GenBank Acc. No.: X15123)
28. SEQ ID NO: 28
   Protein sequence encoding soybean glycinin subunit G3
29. SEQ ID NO: 29
   Nucleic acid sequence encoding sunflower 11S storage protein (G3-D1) (GenBank Acc. No.: M28832)
30. SEQ ID NO: 30
   Protein sequence encoding sunflower 11S storage protein (G3-D1)
31. SEQ ID NO: 31
   Nucleic acid sequence encoding oilseed rape (B. napus) napin (GenBank Acc. No.: J02586)
32. SEQ ID NO: 32
   Protein sequence encoding oilseed rape (B. napus) napin
33. SEQ ID NO: 33
   Nucleic acid sequence encoding Brassica juncea 2S storage protein (GenBank Acc. No.: X65040)
34. SEQ ID NO: 34
   Protein sequence encoding Brassica juncea 2S storage protein
35. SEQ ID NO: 35
   Nucleic acid sequence encoding Brassica oleracea 2S storage protein (GenBank Acc. No.: X65038)
36. SEQ ID NO: 36
   Protein sequence encoding Brassica oleracea 2S storage protein
37. SEQ ID NO: 37
   Nucleic acid sequence encoding Brassica napus cv. Topas napin (GenBank Acc. No.: U04945)
38. SEQ ID NO: 38
   Protein sequence encoding Brassica napus cv. Topas napin
39. SEQ ID NO: 39
   Partial nucleic acid sequence encoding Sinapis alba sin1 storage protein (GenBank Acc. No.: X91799)
40. SEQ ID NO: 40
   Partial protein sequence encoding Sinapis alba sin1 storage protein
41. SEQ ID NO: 41
   Nucleic acid sequence encoding soybean (Glycine max) napin-type 2S albumin 1 (GenBank Acc. No.: U71194)
42. SEQ ID NO: 42
   Protein sequence encoding soybean (Glycine max) napin-type 2S albumin 1
43. SEQ ID NO: 43
   Nucleic acid sequence encoding soybean (Glycine max) 2S albumin (GenBank Acc. No.: AF005030)
44. SEQ ID NO: 44
   Protein sequence encoding soybean (Glycine max) 2S albumin
45. SEQ ID NO: 45
   Nucleic acid sequence encoding Brassica nigra 2S storage protein (GenBank Acc. No.: X65039)
46. SEQ ID NO: 46
   Protein sequence encoding Brassica nigra 2S storage protein
47. SEQ ID NO: 47
   Nucleic acid sequence encoding Sinapis alba sin5 storage protein (GenBank Acc. No.: X91798)
48. SEQ ID NO: 48
   Protein sequence encoding Sinapis alba sin5 storage protein
49. SEQ ID NO: 49
   Nucleic acid sequence encoding sunflower HaG5 2 S albumin (GenBank Acc. No.: X06410)
50. SEQ ID NO: 50
   Protein sequence encoding sunflower HaG5 2 S albumin
51. SEQ ID NO: 51
   Partial nucleic acid sequence encoding sunflower (Helianthus annuus) 2S albumin (GenBank Acc. No.: X76101)
52. SEQ ID NO: 52
   Partial protein sequence encoding sunflower (Helianthus annuus) 2S albumin
53. SEQ ID NO: 53
   Nucleic acid sequence encoding dsRNA for the suppression of Arabidopsis thaliana 12S storage protein AtCru3 (insert of vector pCR2.1-AtCRU3-RNAi)
54. SEQ ID NO: 54
   Ribonucleic acid sequence encoding dsRNA for the suppression of Arabidopsis thaliana 12S storage protein AtCru3
55. SEQ ID NO: 55
   Nucleic acid sequence encoding dsRNA for the suppression of Arabidopsis thaliana 12S storage protein AtCra1
56. SEQ ID NO: 56
   Ribonucleic acid sequence encoding dsRNA for the suppression of Arabidopsis thaliana 12S storage protein AtCra1
57. SEQ ID NO: 57
   Nucleic acid sequence encoding dsRNA for the suppression of Arabidopsis thaliana 2S storage protein At2S2
58. SEQ ID NO: 58
   Ribonucleic acid sequence encoding dsRNA for the suppression of Arabidopsis thaliana 2S storage protein At2S2
59. SEQ ID NO: 59
   Nucleic acid sequence encoding Arabidopsis thaliana 12S cruciferin storage protein (ATCRU3; GenBank Acc. No.: U66916)
60. SEQ ID NO: 60
   Protein sequence encoding Arabidopsis thaliana 12S cruciferin storage protein (ATCRU3)
61. SEQ ID NO: 61
   Nucleic acid sequence encoding A. thaliana 12S storage protein (CRA1; GenBank Acc. No.: M37247)
62. EQ ID NO: 62
   Protein sequence encoding A. thaliana 12S storage protein (CRA1)
63. SEQ ID NO: 63
   Nucleic acid sequence encoding Arabidopsis thaliana 12S storage protein AT5g44120/MLN1_{—}4 (GenBank Acc. No.: AY070730)
64. SEQ ID NO: 64
   Protein sequence encoding Arabidopsis thaliana 12S storage protein AT5g44120/MLN1_{—}4
65. SEQ ID NO: 65
   Nucleic acid sequence encoding Arabidopsis 12S storage protein (CRB; GenBank Acc. No.: X14313; M37248)
66. SEQ ID NO: 66
   Protein sequence encoding Arabidopsis 12S storage protein (CRB)
67. SEQ ID NO: 67
   Nucleic acid sequence encoding Arabidopsis thaliana putative 12S storage protein (from GenBank Acc. No.: AC003027)
68. SEQ ID NO: 68
   Protein sequence encoding Arabidopsis thaliana putative storage protein (Protein_id=“AAD10679.1)
69. SEQ ID NO: 69
   Nucleic acid sequence encoding Arabidopsis thaliana cruciferin 12S storage protein (Atlg03890) (GenBank Acc. No.: AY065432)
70. SEQ ID NO: 70
   Protein sequence encoding Arabidopsis thaliana cruciferin 12S storage protein (Atlg03890)
71. SEQ ID NO: 71
   Nucleic acid sequence encoding Arabidopsis thaliana prohibitin 1 (Atphb1) (GenBank Acc. No.: U66594)
72. SEQ ID NO: 72
   Protein sequence encoding Arabidopsis thaliana prohibitin 1 (Atphb1)
73. SEQ ID NO: 73 oligonucleotide primer OPN1
74. SEQ ID NO: 74 oligonucleotide primer OPN2
75. SEQ ID NO: 75 oligonucleotide primer OPN3
76. SEQ ID NO: 76 oligonucleotide primer OPN4
77. SEQ ID NO: 77 oligonucleotide primer OPN5
78. SEQ ID NO: 78 oligonucleotide primer OPN6
79. SEQ ID NO: 79 oligonucleotide primer OPN7
80. SEQ ID NO: 80 oligonucleotide primer OPN8
81. SEQ ID NO: 81 oligonucleotide primer OPN9
82. SEQ ID NO: 82 oligonucleotide primer OPN10
83. SEQ ID NO: 83
   Nucleic acid sequence encoding sRNAi4-dsRNA for the suppression of a plurality of storage proteins
84. SEQ ID NO: 84
   Ribonucleic acid sequence encoding sRNAi4-dsRNA for the suppression of a plurality of storage proteins
85. SEQ ID NO: 85
   Nucleic acid sequence encoding sRNAi8-dsRNA for the suppression of a plurality of storage proteins
86. SEQ ID NO: 86
   Ribonucleic acid sequence encoding sRNAi8-dsRNA for the suppression of a plurality of storage proteins
87. SEQ ID NO: 87 oligonucleotide primer OPN11
88. SEQ ID NO: 88 oligonucleotide primer OP12
89. SEQ ID NO: 89 oligonucleotide primer OPN13
90. SEQ ID NO: 90 oligonucleotide primer OPN15
91. SEQ ID NO: 91 oligonucleotide primer OPN16
92. SEQ ID NO: 92 oligonucleotide primer OPN17
93. SEQ ID NO: 93
   Nucleic acid sequence encoding Arabidopsis thaliana “globulin-like protein” (GenBank Acc. No.: NM_{—}119834)
94. SEQ ID NO: 94
   Protein sequence encoding Arabidopsis thaliana “globulin-like protein” (Protein_id=“NP_{—}195388.1)
95. SEQ ID NO: 95
   Nucleic acid sequence encoding glycine max 7S seed globulin (GenBank Acc. No.: U59425)
96. SEQ ID NO: 96
   Protein sequence encoding Glycine max 7S seed globulin
97. SEQ ID NO: 97
   Nucleic acid sequence encoding Zea mays 19 kD zein (GenBank Acc. No.: E01144)
98. SEQ ID NO: 98
   Protein sequence encoding Zea mays 19 kD zein
99. SEQ ID NO: 99
   Nucleic acid sequence encoding Zea mays 19 kD alpha zein B1 (GenBank Acc. No.: AF371269)
100. SEQ ID NO: 100
   Protein sequence encoding Zea mays 19 kD alpha zein B1
101. SEQ ID NO: 101
   Nucleic acid sequence encoding Zea mays 19 kD alpha zein B2 (GenBank Acc. No.: AF371270)
102. SEQ ID NO: 102
   Protein sequence encoding Zea mays 19 kD alpha zein B2
103. SEQ ID NO: 103
   Nucleic acid sequence encoding Zea mays 22 kD alpha-zein (GenBank Acc. No.: X61085)
104. SEQ ID NO: 104
   Protein sequence encoding Zea mays 22 kD alpha-zein
105. SEQ ID NO: 105
   Nucleic acid sequence encoding Oryza sativa prolamin (GenBank Acc. No.: AB016503)
106. SEQ ID NO: 106
   Protein sequence encoding Oryza sativa prolamin
107. SEQ ID NO: 107
   Nucleic acid sequence encoding A. sativa avenin (GenBank Acc. NO.: M38446)
108. SEQ ID NO: 108
   Protein sequence encoding A. sativa avenin
109. SEQ ID NO: 109
   Nucleic acid sequence encoding Hordeum vulgare C-hordein (GenBank Acc. No.: M36941)
110. SEQ ID NO: 110
   Protein sequence part 1 encoding Hordeum vulgare C-hordein
111. SEQ ID NO: 111
   Protein sequence part 2 encoding Hordeum vulgare C-hordein
112. SEQ ID NO: 112
   Nucleic acid sequence encoding Triticum aestivum LMW glutenin-1D1 (GenBank Acc. No.: X13306)
113. SEQ ID NO: 113
   Protein sequence encoding Triticum aestivum LMW glutenin-1D1
114. SEQ ID NO: 114
   Binary expression vector for Agrobacterium-mediated plant transformation pSUN2-USP.
115. SEQ ID NO: 115
   Partial nucleic acid sequence encoding Brassica napus homogentisate 1,2-dioxygenase (HGD; EC NO.: 1.13.11.5)
116. SEQ ID NO: 116
   Nucleic acid sequence encoding Arabidopsis thaliana homogentisate 1,2-dioxygenase (HGD; EC NO.: 1.13.11.5)
117. SEQ ID NO: 117
   Protein sequence encoding Arabidopsis thaliana homogentisate 1,2-dioxygenase (HGD; EC NO.: 1.13.11.5)
118. SEQ ID NO: 118
   Nucleic acid sequence encoding Arabidopsis thaliana maleyl-acetoacetate isomerase (MAAI; EC NO.: 5.2.1.2.)
119. SEQ ID NO: 119
   Protein sequence encoding Arabidopsis thaliana maleyl-acetoacetate isomerase (MAAI; EC NO.: 5.2.1.2.)
120. SEQ ID NO: 120
   Nucleic acid sequence encoding Arabidopsis thaliana fumaryl-acetoacetate hydrolase (FAAH; EC NO.: 3.7.1.2)
121. SEQ ID NO: 121
   Protein sequence Arabidopsis thaliana fumaryl-acetoacetate hydrolase (FAAH; EC NO.: 3.7.1.2)
122. SEQ ID NO: 122
   Nucleic acid sequence encoding supression construct 2 (p3300.1-Tocl59-GFP—RNAi)
123. SEQ ID NO: 123 oligonucleotide primer OPN18
124. SEQ ID NO: 124 oligonucleotide primer OPN19
125. SEQ ID NO: 125 oligonucleotide primer OPN20
126. SEQ ID NO: 126 oligonucleotide primer OPN21

### FIGURES

1. FIG. 1: Schematic representation of the storage protein suppression constructs.

Insert from vector pCR2.1-sRNAi4 (1) (cf. Example 2d) and pCR2.1-sRNAi8 (2) (cf. Example 2e) encoding an AtCru3-, AtCRB- and At2S3-expression-suppressing dsRNA.

In the two constructs, the “sense” ribonucleotide sequences and the complementary “antisense” ribonucleotide sequences (symbolized by the upside-down letters) for the individual target genes to be suppressed (AtCru3; AtCRB, At2S3) are arranged differently. Hatched regions (I1, I2 etc.) constitute intron sequences (linkers).

2. FIG. 2A-D: Symbolic representation of various dsRNAs in their secondary structure.

S1, S2, . . . S(n): “sense” ribonucleotide sequence AS1, AS2, . . . AS(n): “antisense” ribonucleotide sequence I: intron sequence

The individual “sense” ribonucleotide sequences and “antisense” ribonucleotide sequences can be arranged in such a way that, first, all “sense” ribonucleotide sequences are arranged one next to the other, thus virtually forming a “sense” strand, whereupon then all “antisense” ribonucleotide sequences are linked with one another to give an “antisense” strand (A and C).

Alternatively, the individual “sense” ribonucleotide sequences and “antisense” ribonucleotide sequences can be arranged in such a way that pairs of in each case complementary “sense” ribonucleotide sequences and “antisense” ribonucleotide sequences are linked with one another (B and D).

“Sense” ribonucleotide sequences and “antisense” ribonucleotide sequences can be linked directly with one another (A and B) or else be separated from one another by further sequences, for example introns (I) (C and D).

3. FIG. 3A-C: Symbolic representation of various dsRNAs in their secondary structure.

S1, S2, . . . S(n): “sense” ribonucleotide sequence

AS1, AS2, . . . AS(n): “antisense” ribonucleotide sequence

SP: “SPACER”

RE: Recognition sequence for ribozyme

R: Ribozyme

“Sense” ribonucleotide sequences and “antisense” ribonucleotide sequences can be separated from one another by further sequences (“SPACER”; SP) (A). The spacer can be for example a recognition sequence for a ribozyme. The corresponding ribozyme can be expressed separately (B) or else likewise be encoded by the spacer (C).

4. FIG. 4: Diagram of the supression construct with the corresponding restriction enzyme cleavage sites:

5. FIG. 5A: Identification of a plant which shows the albino phenotype (left). The phenotype is identical with the ppi2 mutant which is no longer capable of expressing Tocl59. As control, plants with wild-type phenotype grown in parallel.

FIG. 5B: Fluorescence analysis of the plants of FIG. 5A. Excitation of the fluorescence by light in the wavelength range 470-490 nm. The same magnification was chosen as in FIG. 5A.

### EXAMPLES

General Methods:

Unless otherwise specified, all chemicals are obtained from Fluka (Buchs), Merck (Darmstadt), Roth (Karlsruhe), Serva (Heidelberg) and Sigma (Deisenhofen). Restriction enzymes, DNA-modifying enzymes and molecular biology kits were from Amersham-Pharmacia (Freiburg), Biometra (Göttingen), Roche (Mannheim), New England Biolabs (Schwalbach), Novagen (Madison, Wis., USA), Perkin-Elmer (Weiterstadt), Qiagen (Hilden), Stratagen (Amsterdam, Netherlands), Invitrogen (Karlsruhe) and Ambion (Cambridgeshire, United Kingdom). The reagents used were employed in accordance with the manufacturer's instructions.

The chemical synthesis of oligonucleotides can be carried out for example in the known manner using the phosphoamidite method (Voet, Voet, 2nd edition, Wiley Press New York, pages 896-897). The cloning steps carried out for the purpose of the present invention such as, for example, restriction cleavages, agarose gel electrophoresis, purification of DNA fragments, transfer of nucleic acids to nitrocellulose and nylon membranes, linking DNA fragments, transformation of E. coli cells, bacterial cultures, propagation of phages and sequence analysis of recombinant DNA, are carried out as described in Sambrook et al. (1989) Cold Spring Harbor Laboratory Press; ISBN 0-87969-309-6. Recombinant DNA molecules are sequenced using an ABI laser fluorescence DNA sequencer by the method of Sanger (Sanger et al. (1977) Proc Natl Acad Sci USA 74:5463-5467).

### Example 1

### General Methods

The plant Arabidopsis thaliana represents a member of the Higher Plants (spermatophytes). This plant is closely related to other plant species from the Cruciferea family such as, for example, Brassica napus, but also with other plant families of the dicots. Owing to the high degree of homology of its DNA sequences, or polypeptide sequences, Arabidopsis thaliana can be employed as model plant for other plant species.

a) Culture of Arabidopsis Plants

The plants are grown either on Murashige-Skoog medium supplemented with 0.5% sucrose (ogas et al. (1997) Science 277:91-94) or on compost (Focks & Benning (1998) Plant Physiol 118:91-101). To obtain uniform germination and flowering conditions, the seeds are first planted out or sprinkled on compost and then stratified for two days at 4° C. After flowering, the pods are labelled. Then, the pods are harvested according to the labels, at an age of 6 to 20 days after flowering.

b) Isolation of Total RNA and Poly-A⁺ RNA from Plants

RNA, or polyA⁺ RNA is isolated for the generation of suppression constructs. RNA was isolated from pods of Arabidopsis plants using the following protocol: pod material aged 6 to 20 days after flowering was harvested and shock-frozen in liquid nitrogen. Prior to further use, the material was stored at −80° C. 75 mg of the material was ground to a fine powder in a cooled mortar and admixed with 200 μl of the lysis buffer from the Ambion RNAqueos kit. The total RNA was then isolated following the manufacturer's instructions. The RNA was eluted with 50 μl of elution buffer (Ambion) and the concentration was determined by absorption of a 1:100 dilution in a photometer (Eppendorf) at 260 nm. 40 μg/ml RNA corresponds to an absorption of 1. The RNA solutions were brought to a concentration of 1 μg/μl using RNAse-free water. The concentrations were verified by agarose gel electrophoresis. To isolate polyA+ RNA, oligo(dT) cellulose from Amersham Pharmacia was used in accordance with the manufacturer's instructions. RNA or polyA+ RNA was stored at −70° C.

c) Construction of the cDNA Library

To construct the cDNA library from Arabidopsis pod RNA, the first-strand synthesis was obtained using reverse transcriptase from murine leukemia virus (Clontech) and oligo-d(T) primers, while the second-strand synthesis was achieved by incubation with DNA polymerase I, Klenow enzyme and cleavage with RNAse H at 12° C. (2 hours), 16° C. (1 hour) and 22° C. (1 hour). The reaction was stopped by incubation at 65° C. (10 minutes) and subsequently tranferred to ice. Double-stranded DNA molecules were made blunt-ended with T4 DNA polymerase (Roche, Mannheim) at 37° C. (30 minutes). The nucleotides were removed by phenol/chloroform extraction and by means of Sephadex G50 centrifugation columns. EcoRI/XhoI adapters (Pharmacia, Freiburg, Germany) were ligated onto the cDNA ends by means of T4 DNA ligase (Roche, 12° C., overnight), recut with XhoI and phosphorylated by incubation with polynucleotide kinase (Roche, 37° C., 30 minutes). This mixture was subjected to separation on a low-melting agarose gel. DNA molecules over 300 base pairs were eluted from the gel, phenol-extracted, concentrated on Elutip D columns (Schleicher & Schüll, Dassel, Germany), ligated onto vector arms and packaged into lambda-ZAPII phages or lambda-ZAP express phages, using the Gigapack Gold kit (Stratagene, Amsterdam, Netherlands), using the manufacturer's material and instructions.

d) Isolation of Genomic DNA from Plants Such as Arabidopsis thaliana or Brassica napus (CTAB Method)

To isolate genomic DNA from plants such as Arabidopsis thaliana or Brassica napus, approximately 0.25 g of leaf material of young plants in the vegetative stage are comminuted in liquid nitrogen in a pestle and mortar to give a fine powder. The pulverized plant material, together with 1 ml of CTAB I buffer (CTAB: Hexadecyltrimethylammonium bromide, also referred to as Cetyltrimethylammonium bromide; Sigma Cat. NO.: H6269) at a temperature of 65° C. and 20 μl of β-mercaptoethanol, is placed into a prewarmed second mortar and, after complete homogenization, the extract is transferred into a 2 ml Eppendorf vessel and incubated for 1 hour at 65° C. with regular careful mixing. After the mixture has cooled to room temperature, it is extracted in 1 ml of chloroform/octanol (24:1, extracted by shaking with 1M Tris/HCl, pH 8.0) by slowly inverting, and centrifuged for 5 minutes at. 8,500 rpm (7,500×g) and room temperature to achieve phase separation. Thereafter, the aqueous phase is reextracted with 1 ml of chloroform/octanol, centrifuged and mixed carefully by inverting with 1/10 volume of CTAB II buffer which has been prewarmed to 65° C. Thereafter, the mixture is admixed with 1 ml chloroform/octanol mixture (see above) by careful rocking and centrifuged for 5 minutes at 8,500 rpm (7,500×g) and room temperature to reseparate the phases. The aqueous lower phase is transferred into a fresh Eppendorf vessel and the upper organic phase is recentrifuged in a fresh Eppendorf vessel for 15 minutes at 8,500 rpm (7,500×g) and room temperature. The resulting aqueous phase is combined with the aqueous phase of the previous centrifugation step, and all of the mixture is admixed with precisely the same volume of pre-warmed CTAB III buffer. This is followed by incubation at 65° C. until the DNA precipitates in the form of floccules. This may take up to 1 hour or can be carried out overnight by incubation at 37° C. The sediment resulting from the subsequent centrifugation step (5 min, 2000 rpm (500×g), 4° C.) is admixed with 250 μl CTAB IV buffer which has been prewarmed to 65° C. and incubated at 65° C. for at least 30 minutes or until all of the sediment has dissolved. To precipitate the DNA, the solution is subsequently mixed with 2.5 volumes of ice-cold ethanol and incubated for 1 hour at −20° C. Alternatively, the mixture can be mixed with 0.6 volume of isopropanol and centrifuged immediately for 15 minutes at 8,500 rpm (7,500×g) and 4° C., without further incubation. The sedimented DNA is washed by inverting the Eppendorf vessel twice with in each case 1 ml of 80% strength ice-cold ethanol, recentrifuged after each washing step (5 min, 0.8,500 rpm (7,500×g), 4° C.) and subsequently dried in the air for approximately 15 minutes. The DNA is subsequently resuspended in 100 μl of TE with 100 μg/ml RNase and incubated for 30 minutes at room temperature. After a further incubation phase overnight at 4° C., the DNA solution is homogeneous and can be used for subsequent experiments.

Solutions for CTAB:

Solution I (for 200 ml):
   100 mM Tris/HCl pH 8.0 (2.42 g)
   1.4 M NaCl (16.36 g)
   20 mM EDTA (8.0 ml of 0.5 M stock solution)
   2% (w/v) CTAB (4.0 g)

In each case the following is added freshly prior to use: 2% β-mercaptoethanol (20 μl for 1 ml of solution I).
Solution II (for 200 ml):
   0.7 M NaCl (8.18 g)
   10% (w/v) CTAB (20 g)
Solution III (for 200 ml):
   50 mM Tris/HCl pH 8.0 (1.21 g)
   10 mM EDTA (4 ml 0.5 M of 0.5 M stock solution)
   1% (w/v) CTAB (2.0 g)
Solution IV (high-salt TE) (for 200 ml):
   10 mM Tris/HCl pH 8.0 (0.242 g)
   0.1 mM EDTA (40 μl of 0.5 M stock solution)
   1 M NaCl (11.69 g)
Chloroform/octanol (24:1) (for 200 ml):
   192 ml chloroform
   8 ml octanol
   The mixture is extracted twice by shaking with 1 M Tris-HCl, pH 8.0 and stored away from light.

### Example 2

### Generation of Suppression Constructs

Starting from the genomic Arabidopsis thaliana DNA or cDNA, the following fragments of storage protein sequences were amplified via PCR by means of the oligonucleotides listed. The following PCR protocol was employed:
Composition of the PCR mix (50 μl)
   5.00 μl template cDNA or genomic DNA (approx. 1 μg)
   5.00 μl 10× buffer (advantage polymerase)+25 mM MgCl₂
   5.00 μl 2 mM dNTP
   1.25 μl of each primer (10 pmol/μl)
   0.50 μl Advantage polymerase (Clontech)

PCR program: Initial denaturation for 2 minutes at 95° C., then 35 cycles with 45 seconds at 95° C., 45 seconds at 55° C. and 2 minutes at 72° C. Final extension: 5 minutes at 72° C.

a) Starting Vector pCR2.1-AtCRU3-RNAi

Starting from genomic Arabidopsis thaliana DNA, the following oligonucleotide primer pair is used to amplify an exon region with the complete subsequent intron including the splice acceptor sequence of the 12S storage protein AtCRU3, which sequence follows the intron (base pair 1947 to 2603 of the sequence with the GenBank Acc.No: U66916):

ONP1

(SEQ ID NO: 134)

5′-ATAAGAATGCGGCCGCGTGTTCCATTTGGCCGGAAACAAc-3′:
ONP2

(SEQ ID NO: 135)

5′-CCCGGATCCTTCTGTAACATTTGACAAAACATG-3′:

The PCR product is cloned into the pCR2.1-TOPO vector (Invitrogen) following the manufacturer's instructions, resulting in the vector pCR2.1-1, and the sequence is verified.

For the sequence encoding the antisense strand of the dsRNA, the following primer pair is used to amplify only the same exon as above (base pair 1947 to 2384) from Arabidopsis thaliana cDNA:

ONP3

(SEQ ID NO: 136)

5′ATAAGAATGCGGCCGCGTGTTCCATTTGGCCGGAAACAAC -3′:
ONP4

(SEQ ID NO: 137)

5′ATAAGAATGCGGCCGCGGATCCACCCTGGAGAACGCCACGAGTG-3′:

The PCR product is cloned into the pCR2.1-TOPO vector (Invitrogen) following the manufacturer's instructions, resulting in the vector pCR2.1-2, and the sequence is verified.

0.5 μg of vector pCR2.1-1 are incubated with restriction enzyme BamHI (New England Biolabs) for 2 hours following the manufacturer's instructions and then dephosphorylated for 15 minutes with alkaline phosphatase (New England Biolabs). The vector prepared thus (1 μl) is then ligated with the fragment obtained from vector pCR2.1-2. To this end, 0.5 μg of vector pCR2.1-2 is digested for 2 hours with BamHI (New England Biolabs) and the DNA fragments are separated by gel electrophoresis. The 489 bp segment which has been obtained in addition to the vector (3.9 kb) is excised from the gel and purified using the “Gel purification” kit (Qiagen), following the manufacturer's instructions, and eluted with 50 μl of elution buffer. 10 μl of the eluate are ligated with vector pCR2.1-1 (see above) overnight at 16° C. (T4 ligase, New England Biolabs). The ligation products are then transformed into TOP10 cells (Stratagene) following the manufacturer's instructions and a suitable selection takes place. Positive clones are verified with the primer pair ONP1 and ONP2, using PCR. The resulting vector is referred to as pCR2.1-AtCRU3-RNAi. The nucleic acid sequence encoding the dsRNA is described by SEQ ID NO: 105.

b) Starting Vector pCR2.1-AtCRB-RNAi

Using the subsequent oligonucleotide primer pair, an exon region of the 12S storage protein AtCRB (SEQ ID NO: 117 or 118; base pair 601 to 1874 of the sequence with the GenBank Acc. No.: M37248) is amplified from Arabidopsis thaliana cDNA:

ONP5

(SEQ ID NO: 138)

5′ATAAGAATGCGGCCGCGGATCCCTCAGGGTCTTTTCTTGCCCACT-3′:
ONP6

(SEQ ID NO: 139)

5′-CCGCTCGAGTTTACGGATGGAGCCACGAAG-3′:

The PCR product is cloned into the vector pCR2.1-TOPO (Invitrogen) following the manufacturer's instructions, resulting in the vector pCR2.1-3, and the sequence is verified.

For the region which acts as linker, an intron with the relevant splice acceptor and donor sequences of the flanking exons (base pair 1874 to 2117 of the sequence with the GenBank Acc. No.: M37248) is amplified from Arabidopsis thaliana genomic DNA using the following primer pair:

ONP7

(SEQ ID NO: 140)

5′-CCGCTCGAGGTAAGCTCAACAAATCTTTAG-3′:
ONP8

(SEQ ID NO: 141)

5′-ACGCGTCGACGCGTTCTGCGTGCAAGATATT-3′:

The PCR product is cloned into the vector pCR2.1-TOPO (Invitrogen) following the manufacturer's instructions, resulting in the vector pCR2.1-4, and the sequence is verified.

The construct for AtCRB is generated in a similar strategy as described for AtCRU3. Vector pCR2.1-3 is incubated for 2 hours with XhoI (New England Biolabs) and dephosphorylated (alkaline phosphatase, New England Biolabs). Likewise, vector pCR2.1-4 is incubated in the same manner with XhoI, and the gel fragments are separated by gel electrophoresis. The relevant fragments are purified and ligated in the manner described for AtCRU3, resulting, after bacterial transformation, into the vector pCR2.1-AtCRB exon/intron. This vector is incubated for 2 hours with XbaI (NEB), subsequently for 15 minutes with Klenow fragment (NEB), then for 2 hours with SalI and, finally, treated for 15 minutes with alkaline phosphatase (NEB). In parallel, the vector pCR2.1-3 is incubated with BamHI (NEB), then for 15 minutes with Klenow fragment and subsequently for 2 hours with XhoI (NEB). After gel electrophoresis, the exon fragment of AtCRB is isolated, purified and employed for the ligation. The two fragments were then ligated, resulting in the vector pCR2.1-AtCRB-RNAi. The resulting vector is referred to as pCR2.1-AtCRB-RNAi. The nucleic acid sequence which encodes the dsRNA is described by SEQ ID NO: 107.

c) Starting Vector pCR2.1-At2S3-RNAi

Using the following oligonucleotide primer pair, an exon region of the 2S storage protein At2S3 (SEQ ID NO: 3 or 4; base pair 212 to 706 of the sequence with the GenBank Acc. No.: M22035) is amplified:

ONP9

(SEQ ID NO: 142)

5′-ATAAGAATGCGGCCGCGGATCCATGGCTAACAAGCTCTTCCTC
GTC -3′:
ONP10

(SEQ ID NO: 143)

5′-ATAAGAATGCGGCCGCGGATCCCTAGTAGTAAGGAGGGAAGA
AAG-3′:

The PCR product is cloned into the vector pCR2.1-TOPO (Invitrogen) following the manufacturer's instructions, resulting in the vector pCR2.1-5, and the sequence is verified. For the region which acts as linker, the same intron as specified in b), amplified with the primers OPN 7 and OPN 8, is employed. The construct for At2S3 is generated in a similar strategy as described for AtCRU3. Vector pCR2.1-5 is incubated for 2 hours with with XhoI (New England Biolabs) and dephosphorylated (alkaline phosphatase, New England Biolabs). Likewise, vector pCR2.1-3 is incubated in the same manner with XhoI, and the gel fragments are separated by gel electrophoresis. The relevant fragments are purified and ligated in the manner described for AtCRU3, resulting, after bacterial transformation, into the vector pCR2.1-At2S3 exon/intron. This vector is incubated for 2 hours with SalI (NEB), subsequently for 15 minutes with Klenow fragment (NEB) and, treated finally, for 15 minutes with alkaline phosphatase (NEB). In parallel, the vector pCR2.1-5 is incubated with BamHI (NEB) and then for 15 minutes with Klenow fragment. After gel electrophoresis, the exon fragment of At2S3 is isolated, purified and employed for the ligation. The two fragments are then ligated, resulting in the vector pCR2.1-At2S3-RNAi. The nucleic acid sequence which encodes the dsRNA is described by SEQ ID NO: 109.

d) Generation of Super-Supression Construct 1

The vectors pCR2.1-AtCRU3-RNAi and pCR2.1-4 (see above) are incubated for 2 hours at 37° C. with the restriction enzymes XhoI and SalI, the DNA fragments are separated by agarose gel electrophoresis, and both the vector and the PCR insert from pCR2.1-4 are excised and purified with the “Gel purification” kit from Qiagen following the manufacturer's instructions and eluted with 50 μl of elution buffer. 1 μl of the vector eluate and 8 μl of the eluate of the PCR insert from pCR2.1-4 are employed for the ligation, resulting in the construct pCR2.1-sRNAi1. This vector is incubated for 2 hours with the restriction enzyme XhoI and then for 15 minutes with Klenow fragment.

The vector pCR2.1-AtCRB-RNAi (see above) is incubated for 2 hours with the enzyme EcoRI and likewise treated for 15 minutes with Klenow fragment. Both incubation mixtures are separated by gel electrophoresis and either the vector (pCR2.1-sRNAi1) or the insert (from pCR2.1-AtCRB-RNAi) are excised from the agarose gel and the DNA fragments are purified as described above. 1 μl of the eluate of the vector and 8 μl of the eluate from the insert are employed for the ligation and incubated overnight at 4° C. The resulting construct is referred to as pCR2.1-sRNAi2. The resulting vector is incubated with the enzyme XbaI and subsequently with Klenow fragment. The vector pCR2.1-4 is incubated with the enzymes EcoRV and XbaI and subsequently with Klenow fragment. After gel electrophoresis and gel purification, the fragment from pCR2.1-4 is ligated with the vector pCR2.1-sRNAi2, resulting in the construct pCR2.1-sRNAi3. The resulting vector is then incubated for 2 hours with the enzyme ApaI and then for 15 minutes with Klenow fragment. As insert, the vector pCR2.1-At2S3-RNAi is incubated for 2 hours with the enzyme EcoRI and then for 15 minutes with Klenow fragment. After gel electrophoresis and gel purification, the eluates are ligated, resulting in the vector pCR2.1-sRNAi4. The sRNAi4 fragment (SEQ ID NO: 144; cf. FIG. 1(1)), encoding the super-suppressing dsRNA, is then excised from this vector by incubation with HindIII and PvuI and ligated into the binary vector pSUN-USP (SEQ ID NO: 179). The construct serves for the simultaneous suppression of Arabidopsis thaliana storage proteins CRB (SEQ ID NO:4), CRU3 (SEQ ID NO: 112) and At2S3 (SEQ ID NO: 118).

The vector employed pSUN-USP is a binary vector for the transformation of plants, based on pBinAR (Hbfgen and Willmitzer (1990) Plant Science 66: 221-230). A tissue-specific expression in seed can be achieved using the tissue-specific promoter USP promoter.

e) Generation of Super-Supression Construct 2

Starting from Arabidopsis thaliana cDNA, a fragment from the storage protein AtCRU3 (SEQ ID NO: 111, 112) is amplified with the following oligonucleotide primer pair under the PCR conditions stated in Example 2:

(SEQ ID NO: 148)

| | |
|---|---|
| OPN 11: | 5′-AAAAGGCCTGTGTTCCATTTGGCCGGAAACAAC-3′ |

(SEQ ID NO: 149)

| | |
|---|---|
| OPN 12: | 5′-AAAGATATCACCCTGGAGAACGCCACGAGTG-3′. |

The resulting fragment is cloned into the vector pCR2.1-TOPO vector (Invitrogen) following the manufacturer's instructions, resulting in pCR2.1-6, and the sequences are verified.

Starting from Arabidopsis thaliana cDNA, a fragment from the storage protein At253 (SEQ ID NO: 3, 4) is amplified with the following oligonucleotide primer pair under the PCR conditions stated in Example 2:

(SEQ ID NO: 150)

| | |
|---|---|
| OPN 13: | 5′-AAAAGGCCTATGGCTAACAAGCTCTTCCTCGTC-3′ |

(SEQ ID NO: 151)

| | |
|---|---|
| OPN 14: | 5′-AAAGATATCCTAGTAGTAAGGAGGGAAGAAAG-3′. |

The resulting fragment is cloned into the vector pCR2.1-TOPO vector (Invitrogen) following the manufacturer's instructions, resulting in pCR2.1-7, and the sequences are verified.

Starting from pCR2.1-3, pCR2.1-4 (see Example 2) and pCR2.1-6 and pCR2.1-7, the constructs are then ligated with one another as follows: the vector pCR2.1-3 is incubated for 2 hours with EcoRV and subsequently dephosphorylated for 15 minutes with alkaline phosphatase. The vector pCR2.1-6 is incubated for 2 hours with the enzymes StuI and EcoRV and the PCR insert is isolated via gel electrophoresis and gel purification. Vector pCR2.1-3 and insert from pCR2.1-6 are then ligated overnight at 4° C., resulting in the construct pCR2.1-sRNAi5. This vector is then incubated with EcoRV and dephosphorylated and ligated with the StuI/EcoRV-incubated and gel-purified fragment from pCR2.1-7, resulting in the construct pCR2.1-sRNAi6. This vector is then incubated with XhoI and dephosphorylated. The vector pCR2.1-4 is incubated with SalI and XhoI and the insert from pCR2.1-4 is ligated with the prepared vector pCR2.1-sRNAi6, resulting in the construct pCR2.1-sRNAi7. Starting from pCR2.1-sRNAi7, a PCR is carried out with the subsequent primer pair under the conditions stated in Example 2:

(SEQ ID NO: 152)

| | |
|---|---|
| OPN 15: | 5′ CCGCTCGAGCTCAGGGTCTTTTCTTCCCCACT |

(SEQ ID NO: 153)

| | |
|---|---|
| OPN 16: | 5′-CCGGTCGACCTAGTAGTAAGGAGGGAAGAAAG. |

The resulting PCR product is incubated with the enzymes XhoI and SalI. The fragment is then ligated into the vector pCR2.1-sRNAi7 (incubated with XhoI), resulting in the construct pCR2.1-sRNAi8. The sRNAi8 fragment (SEQ ID NO: 146; cf. FIG. 1(2)), encoding the super-suppressing dsRNA, is then excised from this vector by incubation with HindIII and XbaI and ligated into the binary vector pSUN-USP (SEQ ID NO: 179). The construct serves for the simultaneous suppression of Arabidopsis thaliana storage proteins CRB (SEQ ID NO:4), CRU3 (SEQ ID NO: 112) and At2S3 (SEQ ID NO: 118).

### Example 3

### Transformation of Agrobacterium

The Agrobacterium-mediated transformation of plants can be carried out for example using the Agrobacterium tumefaciens strains GV3101 (pMP90) (Koncz and Schell (1986) Mol Gen Genet 204: 383-396) or LBA4404 (Clontech). The transformation can be carried out by standard transformation techniques (Deblaere et al. (1984) Nucl Acids Res 13:4777-4788).

### Example 4

### Plant Transformation

The Agrobacterium-mediated transformation of plants can be carried out using standard transformation and regeneration techniques (Gelvin, Stanton B., Schilperoort, Robert A., Plant Molecular Biology Manual, 2nd ed., Dordrecht: Kluwer Academic Publ., 1995, in Sect., Ringbuc Zentrale Signatur: BT11-P ISBN 0-7923-2731-4; Glick, Bernard R., Thompson, John E., Methods in Plant Molecular Biology and Biotechnology, Boca Raton: CRC Press, 1993, 360 pp., ISBN 0-8493-5164-2).

The transformation of Arabidopsis thaliana by means of Agrobacterium is carried out by the method of Bechthold et al., 1993 (C.R. Acad. Sci. Ser. III Sci. Vie., 316, 1194-1199). Oil-seed rape can be transformed for example by cotyledon or hypocotyl transformation (Moloney et al., Plant Cell Report 8 (1989) 238-242; De Block et al., Plant Physiol. 91 (1989) 694-701). The use of antibiotics for selection of Agrobacterium and plants depends on the binary vector used for the transformation and the Agrobacterium strain. The selection of oilseed rape is usually carried out using kanamycin as selectable plant marker.

Agrobacterium-mediated gene transfer into linseed (Linum usitatissimum) can be carried out using, for example, a technique described by Mlynarova et al. (1994); Plant Cell Report 13:282-285.

The transformation of soybean can be carried out using, for example, a technique described in EP-A-0 0424 047 (Pioneer Hi-Bred International) or in EP-A-0 0397 687, U.S. Pat. No. 5,376,543, U.S. Pat. No. 5,169,770 (University Toledo).

The transformation of plants using particle bombardment, polyethylene-glycol-mediated DNA uptake or via the silicon carbonate fiber technique is described for example by Freeling and Walbot “The maize handbook” (1993) ISBN 3-540-97826-7, Springer Verlag New York).

### Example 5

### Analysis of the Expression of a Recombinant Gene Product in a Transformed Organism

The activity of a recombinant gene product in the transformed host organism was measured at transcription and/or translation level.

A suitable method for determining the amount of transcription of the gene (which indicates the amount of RNA available for the translation of the gene product) is to carry out a Northern blot as detailed hereinbelow (by way of reference, see Ausubel et al. (1988) Current Protocols in Molecular Biology, Wiley: New York, or the abovementioned example section), where a primer which is designed in such a way that it binds to the gene of interest is labeled with a detectable label (usually a radioactive label or chemiluminescent label) so that, when the total RNA of a culture of the organism is extracted, separated on a gel, transferred to a stable matrix and incubated with this probe, the binding and the extent of the binding of the probe indicate the presence and also the amount of the mRNA for this gene. This information also indicates the degree of transcription of the transformed gene. Cellular total RNA can be prepared from cells, tissues or organs in a plurality of methods, all of which are known in the art, such as, for example, the method described by Bormann, E. R., et al. (1992) Mol. Microbiol. 6:317-326.

Northern Hybridization:

To carry out the RNA hybridization, 20 μg of total RNA or 1 μg of poly(A)+ RNA are separated by means of gel electrophoresis in agarose gels with a strength of 1.25% using formaldehyde, as described in Amasino (1986, Anal. Biochem. 152, 304), capillary-blotted to positively charged nylon membranes (Hybond N+, Amersham, Braunschweig) using 10×SSC, immobilized by means of UV light and prehybridized for 3 hours at 68° C. using hybridization buffer (10% dextran sulfate w/v, 1 M NaCl, 1% SDS, 100 mg herring sperm DNA). The DNA probe was labeled with the Highprime DNA labeling kit (Roche, Mannheim, Germany) during the prehybridization, using alpha-³²P-dCTP (Amersham Pharmacia, Braunschweig, Germany). After the labeled DNA probe had been added, the hybridization was carried out in the same buffer at 68° C. overnight. The washing steps were carried out twice for 15 minutes using 2×SSC. and twice for 30 minutes using 1×SSC, 1% SDS, at 68° C. The sealed filters were exposed at −70° C. over a period of 1 to 14 days.

Standard techniques, such as a Western blot, can be employed for assaying the presence or the relative amount of protein translated by this mRNA (see, for example, Ausubel et al. (1988) Current Protocols in Molecular Biology, Wiley: New York). In this method, the cellular total proteins are extracted, separated by means of gel electrophoresis, transferred to a matrix such as nitrocellulose, and incubated with a probe, such as an antibody, which binds specifically to the desired protein. This probe is usually provided with a chemiluminescent or calorimetric label which can be detected readily. The presence and the amount of the label observed indicates the presence and the amount of the desired mutated protein which is present in the cell.

### Example 6

### Analysis of the Effect of the Recombinant Proteins on the Production of the Desired Product

The effect of the genetic modification in plants, fungi, algae, ciliates, or on the production of a desired compound (such as a fatty acid) can be determined by growing the modified micro-organisms or the modified plants under suitable conditions (such as those described above) and analyzing the medium and/or cellular components for the increased production of the desired product (i.e. of lipids or of a fatty acid). These analytical techniques are known to the skilled worker and comprise spectroscopy, thin-layer chromatography, various types of staining methods, enzymatic and microbiological methods, and analytical chromatography such as high-performance liquid chromatography (see, for example, Ullman, Encyclopedia of Industrial Chemistry, Vol. A2, pp. 89-90 and pp. 443-613, VCH: Weinheim (1985); Fallon, A., et al., (1987) “Applications of HPLC in Biochemistry” in: Laboratory Techniques in Biochemistry and Molecular Biology, Vol. 17; Rehm et al. (1993) Biotechnology, Vol. 3, chapter III: “Product recovery and purification”, pp. 469-714, VCH: Weinheim; Belter, P. A., et al. (1988) Bioseparations: downstream processing for Biotechnology, John Wiley and Sons; Kennedy, J. F., and Cabral, J. M. S. (1992) Recovery processes for biological Materials, John Wiley and Sons; Shaeiwitz, J. A., and Henry, J. D. (1988) Biochemical Separations, in: Ullmann's Encyclopedia of Industrial Chemistry, Vol. B3; chapter 11, pp. 1-27, VCH: Weinheim; and Dechow, F. J. (1989) Separation and purification techniques in biotechnology, Noyes Publications).

In addition to the abovementioned methods, plant lipids are extracted from plant material as described by Cahoon et al. (1999) Proc. Natl. Acad. Sci. USA 96 (22):12935-12940, and Browse et al. (1986) Analytic Biochemistry 152:141-145. The qualitative and quantitative analysis of lipids or fatty acids is described in Christie, William W., Advances in Lipid Methodology, Ayr/Scotland: Oily Press (Oily Press Lipid Library; 2); Christie, William W., Gas Chromatography and Lipids. A Practical Guide—Ayr, Scotland: Oily Press, 1989, Repr. 1992, IX, 307 pp. (Oily Press Lipid Library; 1); “Progress in Lipid Research, Oxford: Pergamon Press, 1 (1952)-16 (1977) under the title: Progress in the Chemistry of Fats and Other Lipids CODEN.

In addition to measuring the end product of the fermentation, it is also possible to analyze other components of the metabolic pathways which are used for producing the desired compound, such as intermediates and by-products, in order to determine the overall efficiency of the production of the compound. The analytical methods encompass measurements of the nutrient quantities in the medium (for example sugars, carbohydrates, nitrogen sources, phosphate and other ions), measurements of the biomass composition and of the growth, analysis of the production of customary metabolites of biosynthetic pathways, and measurements of gases produced during fermentation. Standard methods for these measurements are described in Applied Microbial Physiology; A Practical Approach, P. M. Rhodes and P. F. Stanbury, ed., IRL Press, pp. 103-129; 131-163 and 165-192 (ISBN: 0199635773) and references cited therein.

One example is the analysis of fatty acids (abbreviations: FAME, fatty acid methyl esters; GC-MS, gas-liquid chromatography/mass spectrometry; TAG, triacylglycerol; TLC, thin-layer chromatography).

Unambiguous proof for the presence of fatty acid products can be obtained by analyzing recombinant organisms by analytical standard methods: GC, GC-MS or TLC, as described variously by Christie and the references cited therein (1997, in: Advances on Lipid Methodology, fourth edition: Christie, Oily Press, Dundee, 119-169; 1998, Gaschromatographie-Massenspektrometrie-verfahren [gas-chromatographic/mass-spectrometric methods], Lipide 33:343-353).

The material to be analyzed can be disrupted by sonication, milling in the glass mill, liquid nitrogen and milling or other applicable methods. After disruption, the material must be centrifuged. The sediment is resuspended in distilled water, heated for 10 minutes at 100° C., cooled on ice and recentrifuged, followed by extraction in 0.5 M sulfuric acid in methanol with 2% dimethoxypropane for 1 hour at 90° C., which gives hydrolyzed oil and lipid compounds, which give transmethylated lipids. These fatty acid methyl esters are extracted in petroleum ether and finally subjected to GC analysis using a capillary column (Chrompack, WCOT Fused Silica, CP-Wax-52 CB, 25 micrometers, 0.32 mm) at a temperature gradient of between 170° C. and 240° C. for 20 minutes and for 5 minutes at 240° C. The identity of the fatty acid methyl esters obtained must be defined using standards which are available from commercial sources (i.e. Sigma).

The following protocol was used for the oil analysis of the Arabidopsis plants transformed with the suppression constructs: Lipid extraction from the seeds is carried out by the method of Bligh & Dyer (1959) Can J Biochem Physiol 37:911. To this end, 5 mg of Arabidopsis seeds are weighed into 1.2 ml Qiagen microtubes (Qiagen, Hilden) using a Sartorius (Gbttingen) microbalance. The seed material is homogenized with 500 μl chloroform/methanol (2:1; contains mono-C17-glycerol from Sigma as internal standard) in an MM300 Retsch mill from Retsch (Haan) and incubated for 20 minutes at RT. The phases were separated after addition of 500 μl 50 mM potassium phosphate buffer pH 7.5. 50 μl are removed from the organic phase, diluted with 1500 μl of chloroform, and 5 μl are applied to Chromarods SIII capillaries from Iatroscan (SKS, Bechenheim). After application of the samples, they are separated in a first step for 15 mins in a thin-layer chamber saturated with 6:2:2 chloroform:methanol: toluene. After the time has elapsed, the capillaries are dried for 4 minutes at room temperature and then placed for 22 minutes into a thin-layer chamber saturated with 7:3 n-hexane:diethyl ether. After a further drying step for 4 minutes at room temperature, the samples are analyzed in an Iatroscan MK-5 (SKS, Bechenheim) following the method of Fraser & Taggart, 1988 J. Chromatogr. 439:404. The following parameters were set for the measurements: slice width 50 msec, threshold 20 mV, noise 30, skim ratio 0. The data were quantified with reference to the internal standard mono-C17-glycerol (Sigma) and a calibration curve established with tri-C17-glycerol (Sigma), using the program ChromStar (SKS, Beichenheim).

To carry out the quantitative determination of the oil content, seeds of in each case 10 plants of the same independent transgenic line are analyzed. In total, the oil content of 30 transgenic lines of the T1 generation, 10 transgenic lines with in each case 10 plants of the T2 generation and 5 transgenic lines with in each case 10 plants of the T3 lines was determined. The transgenic plants showed a significantly higher oil content than corresponding control plants which had undergone the same treatment.

### Example 7

To assay the functionality of the multiple RNAi constructs, genes ere chosen whose suppression bring about a pronounced phenological effect. An example of such a gene is Toc159. This gene is essential for the development and functionality of chloroplasts in Arabidopsis (Bauer et al. Nature, 403, 203-207). Disruption of this gene leads to chlorophyll-deficient plants hose foliar phenotype is thus pale green to white. This albino phenotype can be distinguished very easily from normal plants.

GFP, the green fluorescent protein from the jellyfish Aequorea victoria was employed as further optical reporter gene. This reporter gene is a frequently used reporter gene in plants (see, for example, Stewart, Plant Cell Rep 2001 20(5):376-82). Arabidopsis thaliana cDNA or from the plasmid pEGFP (BD Clontech, Heidelberg, Genbank Accession U476561), was generated via PCR by means of the oligonucleotides mentioned. The following protocol was employed:

Composition of the PCR mix (50 μl):
5.00 μl template of the cDNA or genomic DNA (approx. 1 μg)
5.00 μl 10× buffer (Advantage polymerase)+25 mM MgCl₂
5.00 μl 2 mM dNTP
1.25 μl for each primer (10 pmol/μl)
0.50 μl Advantage polymerase (Clontech)

PCR program: Initial denaturation for 2 minutes at 95° C., then 35 cycles of 45 seconds at 95° C., 45 seconds at 55° C. and 2 minutes at 72° C. Final extension of 5 minutes at 72° C.

a) Starting vector pGEM-Tocl59: Starting from Arabidopsis cDNA, the following oligonucleotide primer pair was used to amplify a fragment from Toc159 (Genbank Acc. No. T14P8.24):

ONP18

(SEQ ID NO: 123)

5′-CTCGAGGAATTCATGGACTCAAAGTCGGTTACTCCA:
ONP19

(SEQ ID NO: 124)

5′-GGATCCATAAGCAAGCTTTCTCACTCTCCCCATCTGTGGA:

The PCR product was cloned into the vector pGEM-T easy from Promega (Mannheim) following the manufacturer's instructions, resulting in the vector pGEM-Toc159, and the sequence was verified.

b) Starting vector pGEM-GFP: Starting from the plasmid pEGFP (BD lontech, Heidelberg, Genbank Acc.No.: U476561), the following oligonucleotide primer pair was used to amplify a fragment from GFP:

| | |
|---|---|
| ONP20 | |
| 5′-AAGCTTCCAACACTTGTCACTACTTT: | (SEQ ID NO: 125) |
| ONP21 | |
| 5′-GGATCCTTAAAGCTCATCATGTTTGT: | (SEQ ID NO: 126) |

The PCR product was cloned into the vector pGEM-T easy from Promega (Mannheim) following the manufacturer's instructions, resulting in the vector PGEM-GFP, and the sequence was verified.

c) Generation of the construct pGEM-159-GFP. The vector PGEM-GFP was incubated for 2 hours with the restriction enzymes HindIII and BamHI. In parallel, the vector pGEM-Toc159 was incubated with the same restriction enzymes and subsequently then additionally treated for 15 minutes with alkaline phosphatase. The alkaline phosphatase was subsequently inactivated by heating at 95° C. for 10 minutes. The resulting DNA fragments from the two mixtures were separated via agarose gel electrophoresis. The 558 bp fragment from PGEM-GFP and the 3471 bp fragment from pGEM-Toc159 were excised from the gel and purified using the “gel purification” kit (Qiagen) following the manufacturer's instructions. The two fragments were ligated for 2 hours at 16° C. (T4 Ligase, New England Biolabs) and subsequently transformed into E. coli DH5a cells (Stratagen) following the manufacturer's instructions. Positive clones were“−”dentified by PCR using the primer pair OPN1 and OPN4 and subsequently verified by sequencing. The resulting vector was referred to as pGEM-159-GFP.

d) Generation of the supression construct 1: The vector pGEM-159-GFP was firstly incubated with the restriction enzymes XhoI and BamHI, and a further mixture was incubated with BamHI and SalI. The second mixture with BamHI/SalI was subsequently incubated for a further 15 minutes with alkaline phosphatase. The DNA fragments from the two mixtures were separated via agarose gel electrophoresis, and the following fragments were excised: mixture BamHI-XhoI, the 1091 bp fragment; mixture BamHI-SalI, the 4029 bp fragment. Both fragments were isolated from the agarose gel (see above) and then incubated for 2 hours at 16° C. with T4 ligase and subsequently transformed into E. coli DH5a cells (Stratagen). Positive clones were identified by PCR using the primer pair OPN1 and subsequently verified by sequencing. The resulting vector was termed suppression construct 1.

e) Generation of the suppression construct 2: The suppression construct 1 and the vector p3300.1 (Andreas Hilbrunner, PhD thesis ETH Zürich, 2003) were incubated for 2 hours with the restriction enzyme EcoRI. The vector p3300.1 was subsequently treated for 15 minutes with alkaline phosphatase. The two ixtures were mixed and incubated for 2 hours at 16° C. with T4 ligase. The ligation mix was then transformed into E. coli DH5α cells (Stratagen). The resulting suppression construct 2 was then employed for the transformation of Agrobacterium and of plants. The nucleic acid sequence encoding supression construct 2 (p3300.1-Toc159-GFP—RNAi) is shown in SEQ ID NO: 122.

The transformtion of agrobacteria and plants was carried out as described in Example 3 and 4, respectively. To assay the functionality of the suppression construct 2, the latter was transformed into Arabidopsis using the floral transformation method described by Bechtold et al., 1993 (C.R. Acad. Sci. Ser. III Sci. Vie., 316, 1194-1199). Arabidopsis plants of the variety Columbia-0 which already comprise the T-DNA of the bibary vector pBIN-³⁵S-GFP were used as starting material.

In these plants, the green fluorescence of GFP is excited by excitation by ultraviolet light in the wavelength range 470-490 nm, thereby allowing the expression of the transgene which has been introduced. To this end, seedlings 1 week after germination or else leaf segments in older plants were analyzed using the Leica fluorescence microscope MZFLIII. The following parameters were set for the excitation of GFP: mercury lamp HBO 100W/DC, filter GFP3, image processing Leica software. The GFP analysis of green foliar material is made possible specifically by the use of a filter (GFP3) which does not allow transmission above a wavelength of 525 nm. Without this filter, it would not be possible to eliminate the pronounced autofluorescence of the leaf pigment chlorophyll. The Arabidopsis line used for the transformation revealed pronounced GFP expression after analysis under the microscope.

Transformed seeds were sown directly onto the ground and cultivated. After one week a check was made for sprouts having none, or a reduced proportion, of the leaf pigment chlorophyll. Such plants were easy to differetiate owing to their pale green or white phenotype.

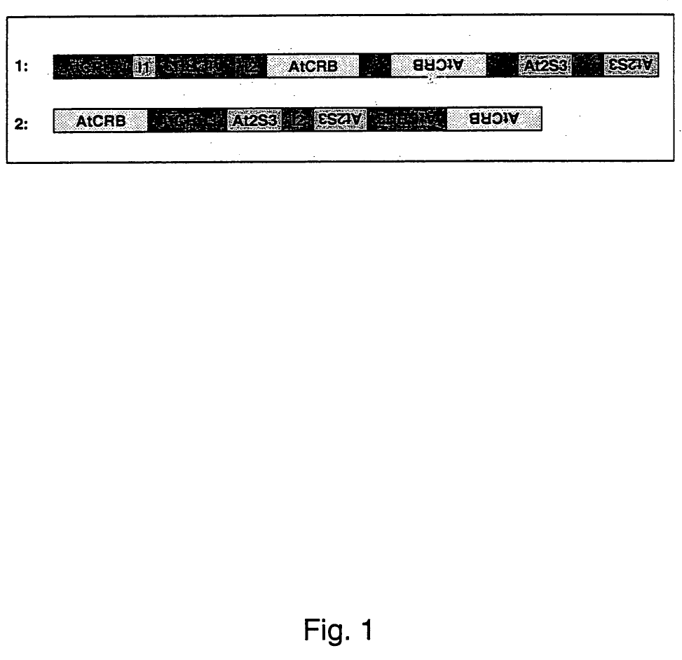

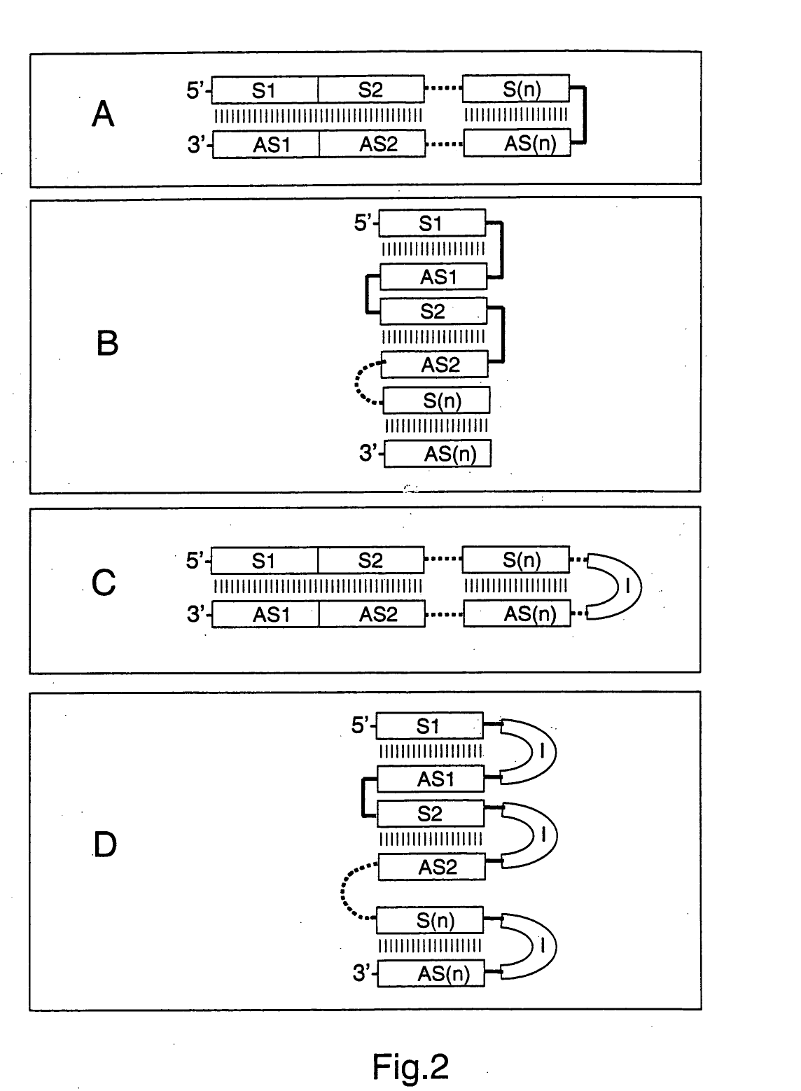

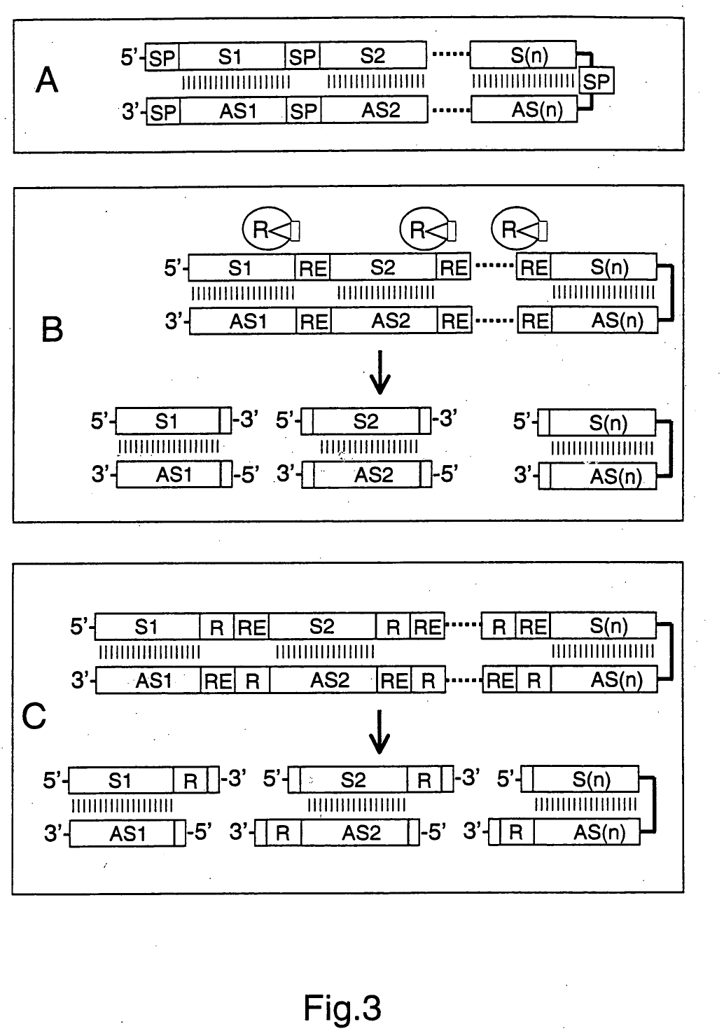

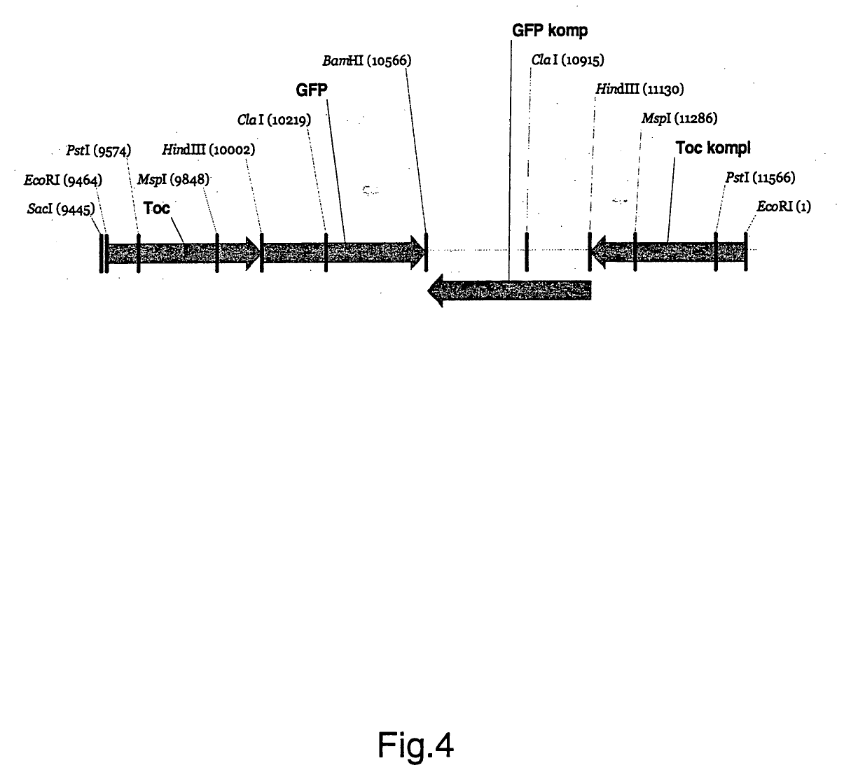

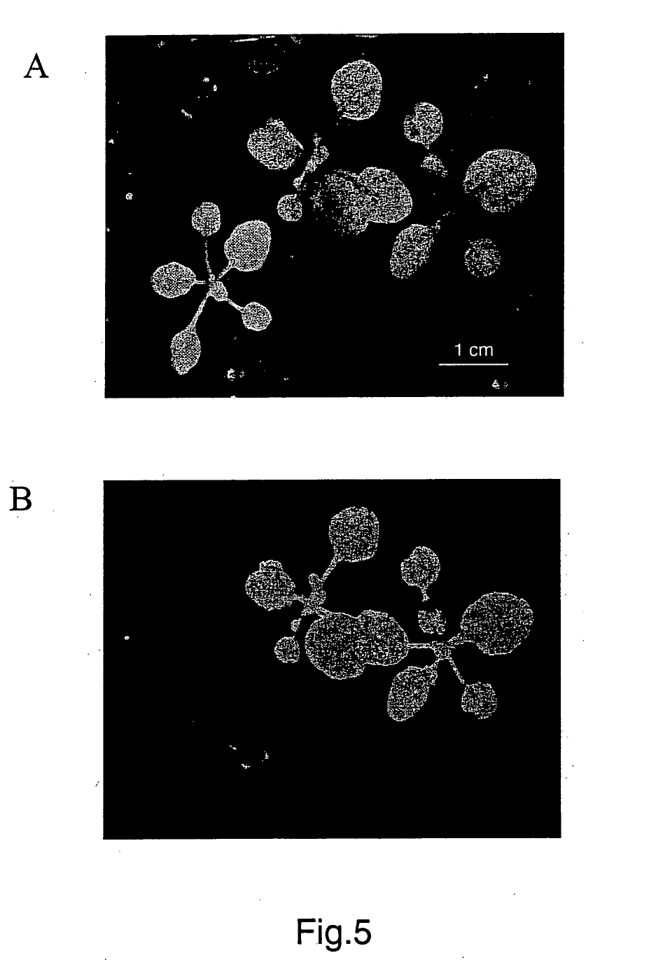

These plants were then studied further under the fluorescence microscope and compared with corresponding green plants which had been grown in parallel. As an example, FIG. 5A shows such a plant which has been identified and whose leaf color markedly differs from plants grown in parallel. The albino phenotype (white leaves) can be attributed to the effect of the Toc159 suppression construct. The untransformed progeny of the plants treated with Agrobacerium suspension do not show the albino phenotype. Thus, the albino phenotype which occurs is a specific effect of the suppression construct which has been introduced.

Analysis of the albino plants under the fluorescence microscope then revealed (FIG. 5B) that no GFP signals were found in those plants. In comparison, clear GFP signals were found in the green plants grown in parallel. The absence of the GFP signal in all the albino plants which have been identified demonstrates the functionality of the suppression construct, since only the plants transformed with the suppression construct no longer show GFP signals. No segregation of the two desired phenotypes was observed. It was thus demonstrated that, by using only one control element (promoter), it was possible to disrupt two genes which have completely different functions and whose expression, in turn, is regulated by differing control elements.

1. A method for reducing expression of at least two different endogenous target genes in a eukaryotic cell or a eukaryotic organism comprising introducing into said eukaryotic cell or said eukaryotic organism, an at least partially double-stranded ribonucleic acid molecule, wherein the double-stranded ribonucleic acid molecule comprises a fully or partially auto-complementary RNA strand, and said RNA strand comprises:
a) at least two sense ribonucleotide sequences, wherein each one of said sense ribonucleotide sequences is essentially identical to at least one part of the sense RNA transcript of each of said at least two different endogenous target genes; and
b) antisense ribonucleotide sequences that are essentially complementary to said sense ribonucleotide sequences, and wherein along said RNA strand the sense ribonucleotide sequences follow one another, followed by a sequential arrangement of the essentially complementary antisense ribonucleotide sequences.

2. The method as claimed in claim 1, wherein the RNA strand forms a single hairpin that has the following primary structure:
5′-S(1)-S(2)-.....S(n)-AS(n)-....AS(2)-AS(1)-3′
in which S is the sense ribonucleotide sequences, AS is the antisense ribonucleotide sequences and n is the number of units which is greater than or equal to two.

3. The method of claim 1, wherein transcribed RNAs of the at least two different target genes whose expression is reduced have less than 90% homology with one another.

4. The method of claim 1, wherein the RNA strand has a length of an even-numbered multiple of 21 or 22 base pairs.

5. The method of claim 1, wherein the ribonucleotide molecule comprises, between at least one sense ribonucleotide sequence and at least one antisense ribonucleotide sequence, which is essentially complementary thereto, a ribonucleotide sequence encoding an intron.

6. The method of claim 1, wherein the at least two of the endogenous target genes are selected from different classes of a storage protein.

7. The method of claim 1, wherein at least one sense ribonucleotide sequence is essentially identical to at least a part of a sense RNA transcript of:
a) a storage protein nucleic acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 59, 61, 63, 65, 67, 69, 71, 93, 95, 97, 99, 101, 103, 105, 107, 109 or 112; or
b) a gene of the homgentisate catabolic pathway having the sequence of SEQ ID NO: 115, 116, 118 or 120; or
c) a gene selected from the group consisting of acetyl transacylases, acyl transport proteins, fatty acid desaturases, malonyl transacylases, β-ketoacyl-ACP synthetases, 3-keto-ACP reductases, enoyl-ACP hydrases, thioesterases, enoyl-ACP reductases, ADP-glucose pyrophosphorylases, phosphorylases, starch synthetases, Q-enzymes, sucrose-6-phosphate synthetases, sucrose-6-phosphate phosphatases. ADP-glucose pyrophosphorylases, branching enzymes, debranching enzymes, amylases, chalcone synthases, chalcone isomerases, phenylalanine ammonialyases, dehydrokempferol(flavone) hydroxylases, dihydroflavonol reductases, dihydroflavanol 2-hydroxylases, flavoriois 3′-hydroxylases, flavonoid 5′-hydroxylases, flavonoid glycosyltransferases, flavonoid methyltransferases, flavonoid acyltransferases, polygalacturonases, cellulases, pectin esterases, β-(1-4)glucanases, β-galactanases, 1-aminocycloproparie-1-carboxylate synthases, phytoene desaturases, cinnamoyl-CoA:NADPH-reductases, cinnamoyl alcohol dehydrogenases, caffeic acid O-methyltransferases, cinnamoyl alcohol dehydrogenases, polyphenol oxidases, homogentisate 1,2-dioxygenases, maleyl-acetoacetate isomerases, fumaryl-acetoacetate hydrolases, N-methylputrescin oxidases, putrescin N-methyltransferases, 7-methylxanthin 3-methyltransferases, 1-methylxanthin 3-methyltransferases and threonin synthases.

8. A ribonucleic acid molecule that has a wholly or partly double-stranded autocomplementary structure and comprises:
a) at least two sense ribonucleotide sequences, wherein at least one of said sense ribonucleotide sequences is essentially identical to at least one part of a sense RNA transcript of an endogenous target gene, but not all sense ribonucleotide sequences are identical to the sense RNA transcript of a single endogenous target gene; and
b) antisense ribonucleotide sequences that are essentially complementary to said sense ribonucleotide sequences, and wherein along said ribonucleic acid molecule sense ribonucleotide sequences follow one another, followed by a sequential arrangement of the essentially complementary antisense ribonucleotide sequences.

9. The ribonucleic acid molecule of claim 8, wherein the RNA strand forms a single hairpin that has the following primary structure:
5′-S(1)-S(2)-.....S(n)-AS(n)-....AS(2)-AS(1)-3′
in which S is the sense ribonucleotide sequences, AS is the antisense ribonucleotide sequences and n is the number of units which is greater than or equal to two.

10. The ribonucleic acid molecule of claim 8, wherein transcribed RNAs of at least two target genes whose expression is reduced have less than 90% homology with one another.

11. A transgenic expression cassette comprising, in operable linkage with a promotor, a nucleic acid sequence encoding the double-stranded ribonucleic acid molecule of claim 8, wherein the ribonucleic acid molecule is formed by a single RNA strand.

12. A transgenic vector comprising the transgenic expression cassette of claim 11.

13. A transgenic organism comprising the transgenic expression cassette of claim 11.

14. The transgenic organism of claim 13 selected from the group consisting of bacteria, yeast, nonhuman animals, plants and combinations thereof.

15. The transgenic organism of claim 13, wherein the organism is selected from the group consisting of agriculturally useful plants.

16. A method for preparation of pharmaceuticals comprising obtaining the ribonucleotide molecule of claim 8.

17. The method of claim 16, wherein at least one of the following characteristics is achieved in plants:
a) improved protection against abiotic stress factors;
b) modification of the composition or content of fatty acids, lipids or oils;
c) modification of carbohydrate composition;
d) modification of color or pigmentation;
e) reduction of storage protein content;
f) obtaining a resistance to plant pathogens;
g) prevention of stem break;
h) delay of fruit maturation;
i) achieving male sterility;
j) reduction of undesired or toxic plant constituents;
k) delay of senescence symptoms;
l) modification of lignification or lignin content;
m) modification of fiber content in foodstuffs or fiber quality in cotton;
n) reduction of susceptibility to bruising;
o) enhancement of vitamin E biosynthesis;
p) reduction of nicotin content, caffeine content or theophyllin content; or
q) increase in methionine content by reducing threonine biosynthesis.

18. The ribonucleotide of claim 8, wherein at least one of the double-stranded RNA structures has a length of an even-numbered multiple of 21 or 22 base pairs.

19. The ribonucleotide of claim 8, wherein the ribonucleotide molecule comprises, between at least one sense ribonucleotide sequence and at least one antisense ribonucleotide sequence which is essentially complementary thereto, a ribonucleotide sequence encoding an intron.

20. The ribonucleotide of claim 8, wherein at least two of the endogenous target genes are selected from different classes of a storage protein.

21. The ribonucleotide of claim 20, wherein the storage protein is selected from the group consisting of albumins, globulins, 115/125 globulins, zein prolamins and combinations thereof.

22. The ribonucleotide of claim 8, wherein at least one sense ribonucleotide sequence is essentially identical to at least a part of a sense RNA transcript of:
a) a storage protein nucleic acid sequence of SEQ ID NO: 1, 3, 5, 7, 9, 11, 13, 15, 17, 19, 21, 23, 25, 27, 29, 31, 33, 35, 37, 39, 41, 43, 45, 47, 49, 51, 59, 61, 63, 65, 67, 69, 71, 93, 95, 97, 99, 101, 103, 105, 107, 109 or 112; or
b) a gene of the homgentisate catabolic pathway having the sequence of SEQ ID NO: 115, 116, 118 or 120; or
c) a gene selected from the group consisting of acetyl transacylases, acyl transport proteins, fatty acid desaturases, malonyl transacylases, β-ketoacyl-ACP synthetases, 3-keto-ACP reductases, enoyl-ACP hydrases, thioesterases, enoyl-ACP reductases, ADP-glucose pyrophosphorylases, phosphorylases, starch synthetases, Q-enzymes, sucrose-6-phosphate synthetases, sucrose-6-phosphate phosphatases. ADP-glucose pyrophosphorylases, branching enzymes, debranching enzymes, amylases, chalcone synthases, chalcone isomerases, phenylalanine ammonialyases, dehydrokempferol(flavone) hydroxylases, dihydroflavonol reductases, dihydroflavanol 2-hydroxylases, flavoriois 3′-hydroxylases, flavonoid 5′-hydroxylases, flavonoid glycosyltransferases, flavonoid methyltransferases, flavonoid acyltransferases, polygalacturonases, cellulases, pectin esterases, β-(1-4)glucanases, β-galactanases, 1-aminocycloproparie-1-carboxylate synthases, phytoene desaturases, cinnamoyl-CoA:NADPH-reductases, cinnamoyl alcohol dehydrogenases, caffeic acid O-methyltransferases, cinnamoyl alcohol dehydrogenases, polyphenol oxidases, homogentisate 1,2-dioxygenases, maleyl-acetoacetate isomerases, fumaryl-acetoacetate hydrolases, N-methylputrescin oxidases, putrescin N-methyltransferases, 7-methylxanthin 3-methyltransferases, 1-methylxanthin 3-methyltransferases and threonin synthases.

23. The method of claim 6, wherein the storage protein is selected from the group consisting of albumins, globulins, 115/125 globulins, zein prolamins and combinations thereof.

24. A method for preparation of pharmaceuticals comprising obtaining the transgenic expression cassette of claim 11.

25. A method for preparation of pharmaceuticals comprising obtaining the transgenic organism of claim 13.


SEQUENCE LISTING (126 sequences)

SEQ ID NO: 1 - DNA, 495 bp, Arabidopsis thaliana
  CDS (1)..(492): albumins 2S subunit 1
atggcaaacaagttgttcctcgtctgcgcagctctcgctctctgcttcctcctcaccaacgcttccatctaccgcaccgtcgttgagttcgaagaagatgacgccactaaccccataggcccaaaaatgaggaaatgccgcaaggagtttcagaaagaacaacacctaagagcttgccagcaattgatgctccagcaagcaaggcaaggccgtagcgatgagtttgatttcgaagacgacatggagaacccacagggacaacagcaggaacaacagctattccagcagtgctgcaacgagcttcgccaggaagagccagattgtgtttgccccaccttgaaacaagctgccaaggccgttagactccagggacagcaccaaccaatgcaagtcaggaaaatttaccagacagccaagcacttgcccaacgtttgcgacatcccgcaagttgatgtttgtcccttcaacatcccttcattcccttctttctactaa

SEQ ID NO: 2 - protein, 164 aa, Arabidopsis thaliana
MANKLFLVCAALALCFLLTNASIYRTVVEFEEDDATNPIGPKMRKCRKEFQKEQHLRACQQLMLQQARQGRSDEFDFEDDMENPQGQQQEQQLFQQCCNELRQEEPDCVCPTLKQAAKAVRLQGQHQPMQVRKIYQTAKHLPNVCDIPQVDVCPFNIPSFPSFY

SEQ ID NO: 3 - DNA, 495 bp, Arabidopsis thaliana
  CDS (1)..(492): albumins 2S subunit 3
atggctaacaagctcttcctcgtctgcgcaactctcgccctctgcttcctcctcaccaacgcttccatctaccgcaccgttgtcgaattcgaagaagatgacgccagcaaccccgtaggtccaagacagagatgccagaaggagtttcagcaatcacaacacctaagagcttgccagagatggatgagcaagcaaatgaggcaaggacgtggtggtggtccttccctcgacgatgagttcgatttcgagggcccccagcagggataccagctactccagcagtgctgcaacgagcttcgccaggaagagccagtttgcgtttgccccaccttgaaacaagctgccagggcagttagcctccagggacagcacggaccattccaatccaggaaaatttaccagtcagctaagtacttgcctaacatttgcaagatccagcaagttggtgaatgtcccttccagaccaccatccctttcttccctccttactactag

SEQ ID NO: 4 - protein, 164 aa, Arabidopsis thaliana
MANKLFLVCATLALCFLLTNASIYRTVVEFEEDDASNPVGPRQRCQKEFQQSQHLRACQRWMSKQMRQGRGGGPSLDDEFDFEGPQQGYQLLQQCCNELRQEEPVCVCPTLKQAARAVSLQGQHGPFQSRKIYQSAKYLPNICKIQQVGECPFQTTIPFFPPYY

SEQ ID NO: 5 - DNA, 513 bp, Arabidopsis thaliana
  CDS (1)..(510): albumins 2S subunit 2
atggcaaacaagctcttcctcgtctgcgcaactttcgccctctgcttcctcctcaccaacgcttccatctaccgcactgttgtcgagttcgacgaagatgacgccagcaaccccatgggcccaagacagaaatgtcagaaggagtttcagcaatcacagcacctaagagcttgccagaaattgatgcgcatgcaaatgaggcaaggccgtggtggtggtccctccctcgacgatgagttcgatttggaagacgacatcgagaacccacaaggcccccagcagggacaccagatcctccagcagtgctgcagcgagcttcgccaggaagagccagtttgtgtttgccccaccttgagacaagctgccagggccgttagcctccagggacaacacggaccattccaatccaggaaaatttacaagacagctaagtacttgcctaacatttgcaagatccagcaagttggtgaatgccccttccagaccaccatccctttcttccctccttactaa

SEQ ID NO: 6 - protein, 170 aa, Arabidopsis thaliana
MANKLFLVCATFALCFLLTNASIYRTVVEFDEDDASNPMGPRQKCQKEFQQSQHLRACQKLMRMQMRQGRGGGPSLDDEFDLEDDIENPQGPQQGHQILQQCCSELRQEEPVCVCPTLRQAARAVSLQGQHGPFQSRKIYKTAKYLPNICKIQQVGECPFQTTIPFFPPY

SEQ ID NO: 7 - DNA, 501 bp, Arabidopsis thaliana
  CDS (1)..(498): albumins 2S subunit 4
atggcgaacaagctcttcctcgtctgcgcagctctcgccctgtgtttcatcctcaccaacgcttccgtctatcgcaccgttgtcgagttcgacgaagatgacgccagtaaccccataggcccaatacagaaatgtcagaaggagtttcagcaagaccagcacctaagagcttgccagagatggatgcgcaagcaaatgtggcaaggacgtggtggtggtccttccctcgacgatgagttcgatatggaagacgacatcgagaacccgcagagacgacagctactccagaagtgctgcagcgagcttcgccaagaagagccagtttgcgtttgccccaccttgagacaagctgccaaggccgttagattccagggacagcaacaccaaccagagcaagtcaggaaaatttaccaggcagctaagtacttgcctaacatttgcaaaatccagcaagttggtgtttgccccttccagatcccttcaatcccttcttactactaa

SEQ ID NO: 8 - protein, 166 aa, Arabidopsis thaliana
MANKLFLVCAALALCFILTNASVYRTVVEFDEDDASNPIGPIQKCQKEFQQDQHLRACQRWMRKQMWQGRGGGPSLDDEFDMEDDIENPQRRQLLQKCCSELRQEEPVCVCPTLRQAAKAVRFQGQQHQPEQVRKIYQAAKYLPNICKIQQVGVCPFQIPSIPSYY

SEQ ID NO: 9 - DNA, 1473 bp, Brassica napus
  CDS (1)..(1470): cruciferin
atggctcggctctcatctcttctctctttttccttagcacttttgatctttctccatggctctacagctcaacagtttccaaacgagtgtcagctagaccagctcaatgcactggagccgtcacacgtacttaaggctgaggctggtcgcatcgaggtgtgggaccaccacgctcctcagctacgttgctctggtgtctcctttgtacgttacatcatcgagtctaagggtctctacttgccctctttctttagcaccgcgaagctctccttcgtggctaaaggagaaggtcttatggggagagtggtccctggatgcgccgagacattccaggactcatcagtgtttcaaccaagcggtggtagcccctcgggagaaggtcagggccaaggacaacaaggtcagggccaaggccaccaaggtcaaggccaaggacaacagggccaacaaggtcagcaaggacaacagagtcaaggccagggcttccgtgatatgcaccagaaagtggagcacataaggactggggacaccatcgctacacatcccggtgtagcccaatggttctacaacgacggaaaccaaccacttgtcatcgtttccgtcctcgatttagccagccaccagaatcagctcgaccgcaacccaaggccattttacttagccggaaacaacccacaaggccaagtatggatagaaggacgcgagcaacagccacaaaagaacatccttaatggcttcacaccagaggttcttgctaaagctttcaagatcgatgttaggacagcgcaacaacttcagaaccagcaagacaaccgtggaaacattatccgagtccaaggcccattcagtgtcattaggccgcctttgaggagtcagagaccgcaggagacagaagttaacggtttagaagagaccatatgcagcgcgaggtgcaccgataacctcgatgacccatctaatgctgacgtatacaagccacagctcggttacatcagcactctgaacagctatgatctccccatccttcgcttccttcgtctctcagccctccgtggatctatccgtcaaaacgcgatggtgcttccacagtggaacgcaaacgcaaacgcggttctctacgtgacagacggggaagcccatgtgcaggtggttaacgacaacggtgacagagtgttcgacggacaagtctctcaaggacagctactttccataccacaaggtttctccgtggtgaaacgcgcaacaagcgaacagttccggtggatcgagttcaagacaaacgcaaacgcacagatcaacacacttgctggacgaacctcggtcttgagaggtttaccattagaggtcatatccaatgggtaccaaatctcactcgaagaagcaagaagggttaagttcaacacgatcgagaccactttgacgcacagcagtggcccagctagctacggagggccaaggaaggctgatgcttaa

SEQ ID NO: 10 - protein, 490 aa, Brassica napus
MARLSSLLSFSLALLIFLHGSTAQQFPNECQLDQLNALEPSHVLKAEAGRIEVWDHHAPQLRCSGVSFVRYIIESKGLYLPSFFSTAKLSFVAKGEGLMGRVVPGCAETFQDSSVFQPSGGSPSGEGQGQGQQGQGQGHQGQGQGQQGQQGQQGQQSQGQGFRDMHQKVEHIRTGDTIATHPGVAQWFYNDGNQPLVIVSVLDLASHQNQLDRNPRPFYLAGNNPQGQVWIEGREQQPQKNILNGFTPEVLAKAFKIDVRTAQQLQNQQDNRGNIIRVQGPFSVIRPPLRSQRPQETEVNGLEETICSARCTDNLDDPSNADVYKPQLGYISTLNSYDLPILRFLRLSALRGSIRQNAMVLPQWNANANAVLYVTDGEAHVQVVNDNGDRVFDGQVSQGQLLSIPQGFSVVKRATSEQFRWIEFKTNANAQINTLAGRTSVLRGLPLEVISNGYQISLEEARRVKFNTIETTLTHSSGPASYGGPRKADA

SEQ ID NO: 11 - DNA, 1467 bp, Brassica napus
  CDS (1)..(1464): cruciferin
atggctcggctctcatctcttctctctttttccttagcacttttgacttttctccatggctctacagctcaacagtttccaaacgagtgtcagctagaccagctcaatgcactggagccgtcacacgtacttaaggctgaggctggtcgcatcgaggtgtgggaccaccacgctcctcagctacgttgctctggtgtctcctttgtacgttacatcatcgagtctaagggtctctacttgccctctttctttagcaccgcgaggctctccttcgtggctaaaggagaaggtcttatggggagagtggtcctgtgcgccgagacattccaggactcatcagtgtttcaaccaagcggtggtagccccttcggagaaggtcagggccaaggacaacaaggtcagggccaaggccaccaaggtcaaggccaaggacaacagggccaacaaggtcagcaaggacaacagagtcaaggccagggtttccgtgatatgcaccagaaagtggagcacataaggactggggacaccatcgctacacatcccggtgtagcccaatggttctacaacgacggaaaccaaccacttgtcatcgtttccgtcctcgatttagccagccaccagaatcagctcgaccgcaacccaaggccattttacttagccggaaacaacccacaaggccaagtatggatagaaggacgcgagcaacagccacaaaagaacatccttaatggcttcacaccagaggttcttgctaaagctttcaagatcgatgttaggacagcgcaacaacttcagaaccagcaagacaaccgtggaaacattatccgagtccaaggcccattcagtgtcattaggccgcctttgaggagtcagagaccgcaggaggaagttaacggtttagaagagaccatatgcagcgcgaggtgcaccgataacctcgatgacccatctaatgctgacgtatacaagccacagctcggttacatcagcactctgaacagctatgatctccccatccttcgcttccttcgtctctcagccctccgtggatctatccgtcaaaacgcgatggtgcttccacagtggaacgcaaacgcaaacgcggttctctacgtgacagacggggaagcccatgtgcaggtggttaacgacaacggtgacagagtgttcgacggacaagtctctcaaggacagctactttccataccacaaggtttctccgtggtgaaacgcgcaacaagcgaacagttccggtggatcgagttcaagacaaacgcaaacgcacagatcaacacacttgctggacgaacctcggtcttgagaggtttaccattagaggtcatatccaatgggtaccaaatctcactcgaagaagcaagaagggttaagttcaacacgatcgagaccactttgacgcacagcagtggcccagctagctacggagggccaaggaaggctgatgcttaa

SEQ ID NO: 12 - protein, 488 aa, Brassica napus
MARLSSLLSFSLALLTFLHGSTAQQFPNECQLDQLNALEPSHVLKAEAGRIEVWDHHAPQLRCSGVSFVRYIIESKGLYLPSFFSTARLSFVAKGEGLMGRVVLCAETFQDSSVFQPSGGSPFGEGQGQGQQGQGQGHQGQGQGQQGQQGQQGQQSQGQGFRDMHQKVEHIRTGDTIATHPGVAQWFYNDGNQPLVIVSVLDLASHQNQLDRNPRPFYLAGNNPQGQVWIEGREQQPQKNILNGFTPEVLAKAFKIDVRTAQQLQNQQDNRGNIIRVQGPFSVIRPPLRSQRPQEEVNGLEETICSARCTDNLDDPSNADVYKPQLGYISTLNSYDLPILRFLRLSALRGSIRQNAMVLPQWNANANAVLYVTDGEAHVQVVNDNGDRVFDGQVSQGQLLSIPQGFSVVKRATSEQFRWIEFKTNANAQINTLAGRTSVLRGLPLEVISNGYQISLEEARRVKFNTIETTLTHSSGPASYGGPRKADA

SEQ ID NO: 13 - DNA, 1491 bp, Brassica napus
  CDS (1)..(1488): cruciferin BnC2
atggctcgactctcgtctcttctctatttttcgataacagttttgatctttctccatggctctacagctcaacagtttccaaacgagtgccaactagaccagctcaatgcgctggagccgtcacacgtacttaaggccgaggctggtcgcatcgaagtgtgggaccaccacgctcctcagctacgctgctctggtgtctccttcgtacgttacataatcgagtctcagggtctatacttgccctctttcttaaataccgcgaacgtctctttcgttgctaaaggacaaggccttatggggagagtggtccctggatgcgctgagactttccaggactcatcagtattccaaccaggcagtggcagccccttcggagaaggtcaaggccaaggtcagcagggtcaggggcaaggtcagggtcagggtcaaggcaagggccaacagggtcaaggcaagggccaacagggtcaatcccagggccaacagggtcaaggtcaaggtttccgtgatatgcaccagaaagtagagcacataaggagcggcgacaccattgctacacatcccggtgtagctcaatggttctacaacaatggaaaccaacctcttgtcatcgttgccgtcatggatttagctagccaccagaaccagcttgaccgcaacccaagccaattttacttagcaggaaaaaacccacaaggccaatcatggctacacggacgagggcaacagccacaaaacaacatccttaatggcttctctccagaggttcttgctcaagcgttcaagatcgatgttaggacagcgcaacaacttcagaaccagcaagataaccggggaaacattgtccgtgtccaaggccccttcggtgttattaggccgccattgaaaagccagagaccacaggagacagaagctaacggtctagaagagaccatatgcagcgcaaggtgcacggataacctcgatgacccatctaacgcggatgtgtataagccacagcttggttacatcagcattcttaacagttatgatctacccatccttcgcgtacttcgcctctcagccctccgtggatcaatccgtcaaaatgcaatggttcttccacagtggaagtcaaagtcaaacgcggttctctacgtgacagacggggaagcccaaatacaggtggttaacgacaacggtgacagagtgttcgatggacaagtctctcaagggcagctactttccattccacaaggattctccgttgtgaaacgcgcaacaagcgatcagttcaggtggatagaattcaagacaaacgcaaacgcccagatcaacactcttgctggacgtacctcagtcatgagaggtttaccattagaggtcatagccaatgggtaccaaatctcacttgaagaagcaagaagggttaagttcaacacaatagagaccactttgacccacagtagtggcccagcgagctacggaaggccaaggaaggctgatgcttga

SEQ ID NO: 14 - protein, 496 aa, Brassica napus
MARLSSLLYFSITVLIFLHGSTAQQFPNECQLDQLNALEPSHVLKAEAGRIEVWDHHAPQLRCSGVSFVRYIIESQGLYLPSFLNTANVSFVAKGQGLMGRVVPGCAETFQDSSVFQPGSGSPFGEGQGQGQQGQGQGQGQGQGKGQQGQGKGQQGQSQGQQGQGQGFRDMHQKVEHIRSGDTIATHPGVAQWFYNNGNQPLVIVAVMDLASHQNQLDRNPSQFYLAGKNPQGQSWLHGRGQQPQNNILNGFSPEVLAQAFKIDVRTAQQLQNQQDNRGNIVRVQGPFGVIRPPLKSQRPQETEANGLEETICSARCTDNLDDPSNADVYKPQLGYISILNSYDLPILRVLRLSALRGSIRQNAMVLPQWKSKSNAVLYVTDGEAQIQVVNDNGDRVFDGQVSQGQLLSIPQGFSVVKRATSDQFRWIEFKTNANAQINTLAGRTSVMRGLPLEVIANGYQISLEEARRVKFNTIETTLTHSSGPASYGRPRKADA

SEQ ID NO: 15 - DNA, 555 bp, Brassica napus
  CDS (1)..(552): cruciferin cru4
ttgtgcacaatgagatgcaccgaaaaccttgatgacccgtcaagtgctgatgtctacaagccatcgctcggatacattagcacactcaacagctacaacctccctatcctcagattcctccgccttagcgctcttcgtggctccatccataacaacgctatggtgctgccgcaatggaacgtgaacgcaaacgcggcactctacgtgacaaaggggaaggctcatatacagatggtgaacgacaacggacaaagagtgtttgaccaagagatctcccagggacagttacttgtcgtgccacaaggcttcgcggtcgtgaaacgtgccacaagccaacagttccagtggatcgagttcaagagcaacgacaacgcacagatcaacacactcgcgggacgcacctcagtcatgagaggtttaccacttgaggttatatccaacgggtatcagatctcaccccaagaagctagaagtgttaagttcagcactcttgagaccacattgactcaaagcagtggtcctatgggctacggtatgcctagagtcgaggcttga

SEQ ID NO: 16 - protein, 184 aa, Brassica napus
LCTMRCTENLDDPSSADVYKPSLGYISTLNSYNLPILRFLRLSALRGSIHNNAMVLPQWNVNANAALYVTKGKAHIQMVNDNGQRVFDQEISQGQLLVVPQGFAVVKRATSQQFQWIEFKSNDNAQINTLAGRTSVMRGLPLEVISNGYQISPQEARSVKFSTLETTLTQSSGPMGYGMPRVEA

SEQ ID NO: 17 - DNA, 1530 bp, Brassica napus
  CDS (1)..(1527): cruciferin cru4
atggttaaagttcctcatctcctcgtcgcaacgttcggggttctcctcgtcctcaacggctgtctcgcaaggcagtcgctaggggttcctcctcagctagggaacgcgtgtaacctcgataacttagacgttctccagcctaccgaaactatcaagagcgaggctggtcgggtcgagtactgggatcacaacaatcctcagatccgatgtgctggtgtctctgtctctcgtgttataatcgaacaaggcggtctctaccttcctaccttcttcagctcccccaaaatttcatacgttgttcaaggaatgggtattagcggaagagtggtccctggatgcgcggaaaccttcatggactcgcagcctatgcaaggacaacaacaaggtcaaccatggcagggacaacaaggacaacagggtcagcagggacaacaaggtcaacagggtcagcagggacaacaaggtcaacagggtcagcagggtcaacagggacagcagggtcagcagcagcaagggttccgtgacatgcaccagaaggtcgaacatgttcgacatggagacatcattgccattactgcaggctcttcccattggatctacaacaccggtgaccagccacttgtcattatctgccttctcgacattgccaactaccaaaaccaactcgaccgcaacccaagaacgttccgtctggccggaaacaacccacagggcggttcccagcagcagcagcaacaacaacagaacatgttgagcgggttcgaccctcaggtcctagcccaggcattgaaaatcgacgttaggttggctcaggagcttcagaaccaacaagacagcagaggaaacatcgttcgtgttaagggacctttccaggttgtgaggccgcctcttagacagccatacgagagtgagcagtggagacacccccgtggcccaccacaaagcccacaagacaacggcttggaggagactatctgcagcatgaggacccacgagaacattgatgacccagcccgtgctgacgtgtataagcccaacctcggccgtgtgactagcgtcaacagctacactttacccatcttgcagtatatcagactcagcgccacccgtggcattctccagggtaatgcgatggtgcttccgaaatacaacatgaacgcgaacgagatcttgtactgcactcaaggacaagcaaggattcaagtggtgaacgacaacggacagaacgtgctggaccagcaggtgcagaagggacagctcgtggtcatcccacaaggattcgcctatgttgtccagtcccaccaaaacaacttcgaatggatttctttcaagacaaacgctaacgcgatggtcagcactttggccggtagaacctcggccttgagggcattgccactagaggtcataaccaacgctttccaaatttctctcgaggaagctagaaggatcaagttcaacacgcttgagaccactttgactcgtgcgcgcggtggacaaccccagttgatcgaggagatagtcgaggcttaa

SEQ ID NO: 18 - protein, 509 aa, Brassica napus
MVKVPHLLVATFGVLLVLNGCLARQSLGVPPQLGNACNLDNLDVLQPTETIKSEAGRVEYWDHNNPQIRCAGVSVSRVIIEQGGLYLPTFFSSPKISYVVQGMGISGRVVPGCAETFMDSQPMQGQQQGQPWQGQQGQQGQQGQQGQQGQQGQQGQQGQQGQQGQQGQQQQGFRDMHQKVEHVRHGDIIAITAGSSHWIYNTGDQPLVIICLLDIANYQNQLDRNPRTFRLAGNNPQGGSQQQQQQQQNMLSGFDPQVLAQALKIDVRLAQELQNQQDSRGNIVRVKGPFQVVRPPLRQPYESEQWRHPRGPPQSPQDNGLEETICSMRTHENIDDPARADVYKPNLGRVTSVNSYTLPILQYIRLSATRGILQGNAMVLPKYNMNANEILYCTQGQARIQVVNDNGQNVLDQQVQKGQLVVIPQGFAYVVQSHQNNFEWISFKTNANAMVSTLAGRTSALRALPLEVITNAFQISLEEARRIKFNTLETTLTRARGGQPQLIEEIVEA

SEQ ID NO: 19 - DNA, 1488 bp, Glycine max
  CDS (1)..(1485): Gycinin A-1a-B-x subunit
atggccaagctagttttttccctttgttttctgcttttcagtggctgctgcttcgctttcagttccagagagcagcctcagcaaaacgagtgccagatccaaaaactcaatgccctcaaaccggataaccgtatagagtcagaaggagggctcattgagacatggaaccctaacaacaagccattccagtgtgccggtgttgccctctctcgctgcaccctcaaccgcaacgcccttcgtagaccttcctacaccaacggtccccaggaaatctacatccaacaaggtaagggtatttttggcatgatatacccgggttgtcctagcacatttgaagagcctcaacaacctcaacaaagaggacaaagcagcagaccacaagaccgtcaccagaagatctataacttcagagagggtgatttgatcgcagtgcctactggtgttgcatggtggatgtacaacaatgaagacactcctgttgttgccgtttctattattgacaccaacagcttggagaaccagctcgaccagatgcctaggagattctatcttgctgggaaccaagagcaagagtttctaaaatatcagcaagagcaaggaggtcatcaaagccagaaaggaaagcatcagcaagaagaagaaaacgaaggaggcagcatattgagtggcttcaccctggaattcttggaacatgcattcagcgtggacaagcagatagcgaaaaacctacaaggagagaacgaaggggaagacaagggagccattgtgacagtgaaaggaggtctgagcgtgataaaaccacccacggacgagcagcaacaaagaccccaggaagaggaagaagaagaagaggatgagaagccacagtgcaagggtaaagacaaacactgccaacgcccccgaggaagccaaagcaaaagcagaagaaatggcattgacgagaccatatgcaccatgagacttcgccacaacattggccagacttcatcacctgacatctacaaccctcaagccggtagcgtcacaaccgccaccagccttgacttcccagccctctcgtggctcagactcagtgctgagtttggatctctccgcaagaatgcaatgttcgtgccacactacaacctgaacgcgaacagcataatatacgcattgaatggacgggcattgatacaagtggtgaattgcaacggtgagagagtgtttgatggagagctgcaagagggacgggtgctgatcgtgccacaaaactttgtggtggctgcaagatcacagagtgacaacttcgagtatgtgtcattcaagaccaatgatacacccatgatcggcactcttgcaggggcaaactcattgttgaacgcattaccagaggaagtgattcagcacactttcaacctaaaaagccagcaggccaggcagataaagaacaacaaccctttcaagttcctggttccacctcaggagtctcagaagagagctgtggcttag

SEQ ID NO: 20 - protein, 495 aa, Glycine max
MAKLVFSLCFLLFSGCCFAFSSREQPQQNECQIQKLNALKPDNRIESEGGLIETWNPNNKPFQCAGVALSRCTLNRNALRRPSYTNGPQEIYIQQGKGIFGMIYPGCPSTFEEPQQPQQRGQSSRPQDRHQKIYNFREGDLIAVPTGVAWWMYNNEDTPVVAVSIIDTNSLENQLDQMPRRFYLAGNQEQEFLKYQQEQGGHQSQKGKHQQEEENEGGSILSGFTLEFLEHAFSVDKQIAKNLQGENEGEDKGAIVTVKGGLSVIKPPTDEQQQRPQEEEEEEEDEKPQCKGKDKHCQRPRGSQSKSRRNGIDETICTMRLRHNIGQTSSPDIYNPQAGSVTTATSLDFPALSWLRLSAEFGSLRKNAMFVPHYNLNANSIIYALNGRALIQVVNCNGERVFDGELQEGRVLIVPQNFVVAARSQSDNFEYVSFKTNDTPMIGTLAGANSLLNALPEEVIQHTFNLKSQQARQIKNNNPFKFLVPPQESQKRAVA

SEQ ID NO: 21 - DNA, 1458 bp, Glycine max
  CDS (1)..(1455): glycinin G2 subunit
atggccaagcttgttctttccctttgtttccttcttttcagtggctgcttcgctctgagagagcaggcacagcaaaatgagtgccagatccaaaagctgaatgccctcaaaccggataaccgtatagagtcggaaggtgggttcattgagacatggaaccctaacaacaagccattccagtgtgccggtgttgccctctctcgctgcacccttaaccgcaatgcccttcgtagaccttcctacaccaacggtccccaggaaatctacatacaacaaggtaatggtatttttggcatgatattcccgggttgtcctagcacttatcaagagccgcaagaatctcagcaacgaggacgaagccagaggccccaagaccgtcaccaaaaggtacatcgcttcagagagggtgatttgatcgcagtgcctactggtgttgcatggtggatgtacaacaatgaagacactcctgttgttgccgtttctattattgacaccaacagcttggagaaccagctcgaccagatgcctaggagattctatcttgctgggaaccaagagcaagagtttctaaaatatcagcagcagcagcaaggaggttcccaaagccagaaaggaaagcaacaagaagaagaaaacgaaggaagcaacatattgagtggcttcgcccctgaattcttgaaagaagcgttcggcgtgaacatgcagatagtgagaaacctacaaggtgagaacgaagaggaggatagtggagccattgtgacagtgaaaggaggtctaagagtcacagctccagccatgaggaagccacagcaagaagaagatgatgatgatgaggaagagcagccacagtgcgtggagacagacaaaggttgccaacgccaaagcaaaaggagcagaaatggcattgatgagaccatttgcacaatgagacttcgccaaaacattggtcagaattcatcacctgacatctacaaccctcaagctggtagcatcacaaccgccaccagccttgacttcccagccctctggcttctcaaactcagtgcccagtatggatcactccgcaagaatgctatgttcgtgccacactacaccctgaacgcgaacagcataatatacgcattgaatgggcgggcattggtacaagtggtgaattgcaatggtgagagagtgtttgatggagagctgcaagagggaggggtgctgatcgttccacaaaactttgcggtggctgcaaaatcccagagcgataactttgagtatgtgtcattcaagaccaatgatagaccctcgatcggaaaccttgcaggggcaaactcattgttgaacgcattgccagaggaagtgattcagcacacttttaacctaaagagccagcaggccaggcaggtgaagaacaacaaccctttcagcttccttgttccacctcaggagtctcagaggagagctgtggcttag

SEQ ID NO: 22 - protein, 485 aa, Glycine max
MAKLVLSLCFLLFSGCFALREQAQQNECQIQKLNALKPDNRIESEGGFIETWNPNNKPFQCAGVALSRCTLNRNALRRPSYTNGPQEIYIQQGNGIFGMIFPGCPSTYQEPQESQQRGRSQRPQDRHQKVHRFREGDLIAVPTGVAWWMYNNEDTPVVAVSIIDTNSLENQLDQMPRRFYLAGNQEQEFLKYQQQQQGGSQSQKGKQQEEENEGSNILSGFAPEFLKEAFGVNMQIVRNLQGENEEEDSGAIVTVKGGLRVTAPAMRKPQQEEDDDDEEEQPQCVETDKGCQRQSKRSRNGIDETICTMRLRQNIGQNSSPDIYNPQAGSITTATSLDFPALWLLKLSAQYGSLRKNAMFVPHYTLNANSIIYALNGRALVQVVNCNGERVFDGELQEGGVLIVPQNFAVAAKSQSDNFEYVSFKTNDRPSIGNLAGANSLLNALPEEVIQHTFNLKSQQARQVKNNNPFSFLVPPQESQRRAVA

SEQ ID NO: 23 - DNA, 1689 bp, Glycine max
  CDS (1)..(1686): glycinin A5A4B3 subunits
atggggaagcccttcactctctctctttcttccctttgcttgctactcttgtcgagtgcatgctttgctattagctccagcaagctcaacgagtgccaactcaacaacctcaacgcgttggaacccgaccaccgcgttgagtccgaaggtggtttgattcaaacatggaactctcaacaccctgagctgaaatgcgccggtgtcactgtttccaaactcaccctcaaccgcaatggcctccactcgccatcttactcaccttatccccggatgatcatcatcgcccaagggaaaggagcacttggagttgcaattccaggatgtcctgagacgtttgaggagccacaagaacaatcaaacagaagaggctcaaggtcgcagaagcagcagctacaggacagtcaccagaagattcgtcacttcaatgaaggagacgtactcgtgattcctcctagtgttccttactggacctataacactggcgatgaaccagttgttgccatcagtcttcttgacacctctaacttcaataaccagcttgatcaaacccctagggtattttaccttgctgggaacccagatatagagtacccagagaccatgcaacaacaacaacagcagaaaagtcatggtggacgcaagcaggggcaacaccagcaggaggaagaggaagaaggtggcagcgtgctcagtggcttcagcaaacacttcttggcacaatccttcaacaccaacgaggacatagctgagaaacttgagtctccagacgacgaaaggaagcagatcgtgacagtggaaggaggtctcagcgttatcagccccaagtggcaagaacaacaagatgaagatgaagatgaagacgaagatgatgaagatgaacaaattccctctcaccctcctcgccgaccaagccatggaaagcgtgaacaagacgaggacgaggacgaagatgaagataaacctcgtcctagtcgaccaagccaaggaaagcggaacaagacaggacaggacgaggacgaagatgaagatgaagatcaacctcgcaagagccgcgaatggagatcgaaaaagacacaacccagaagacctagacaagaagaaccacgtgaaagaggatgcgagacaagaaacggggttgaggaaaatatctgcaccttgaagcttcacgagaacattgctcgcccttcacgcgctgacttctacaaccctaaagctggtcgcattagtaccctcaacagcctcaccctcccagccctccgccaattccaactcagtgcccaatatgttgtcctctacaagaatggaatttactctccacattggaatctgaatgcaaacagtgtgatctatgtgactcgaggacaaggaaaggttagagttgtgaactgccaagggaatgcagtgttcgacggtgagcttaggaggggacaattgctggtggtaccacagaacttcgtggtggcggagcaagccggagaacaaggattcgaatacatagtattcaagacacaccacaacgcagtcactagctacttgaaggatgtgtttagggcaattccctcagaggttcttgcccattcttacaaccttcgacagagtcaagtgtctgagcttaagtatgaaggaaattggggtcctttggtcaaccctgagtctcaacaaggctcaccccgtgttaaagtcgcataa

SEQ ID NO: 24 - protein, 562 aa, Glycine max
MGKPFTLSLSSLCLLLLSSACFAISSSKLNECQLNNLNALEPDHRVESEGGLIQTWNSQHPELKCAGVTVSKLTLNRNGLHSPSYSPYPRMIIIAQGKGALGVAIPGCPETFEEPQEQSNRRGSRSQKQQLQDSHQKIRHFNEGDVLVIPPSVPYWTYNTGDEPVVAISLLDTSNFNNQLDQTPRVFYLAGNPDIEYPETMQQQQQQKSHGGRKQGQHQQEEEEEGGSVLSGFSKHFLAQSFNTNEDIAEKLESPDDERKQIVTVEGGLSVISPKWQEQQDEDEDEDEDDEDEQIPSHPPRRPSHGKREQDEDEDEDEDKPRPSRPSQGKRNKTGQDEDEDEDEDQPRKSREWRSKKTQPRRPRQEEPRERGCETRNGVEENICTLKLHENIARPSRADFYNPKAGRISTLNSLTLPALRQFQLSAQYVVLYKNGIYSPHWNLNANSVIYVTRGQGKVRVVNCQGNAVFDGELRRGQLLVVPQNFVVAEQAGEQGFEYIVFKTHHNAVTSYLKDVFRAIPSEVLAHSYNLRQSQVSELKYEGNWGPLVNPESQQGSPRVKVA

SEQ ID NO: 25 - DNA, 1551 bp, Glycine max
  CDS (1)..(1548): glycinin A3-B4 subunit
atggggaagcccttcttcactctctctctttcttccctttgcttgctactcttgtcgagtgcatgctttgctattacctccagcaagttcaacgagtgccaactcaacaacctcaacgcgttggaacccgaccaccgcgttgagtccgaaggtggtcttattgaaacatggaactctcaacaccctgagctgcaatgcgccggtgtcactgtttccaaacgcaccctcaaccgcaacggctcccacttgccatcttacttaccttatccccaaatgatcattgtcgttcaagggaagggagcaattggatttgcatttccgggatgtcccgagacgtttgagaagccacaacaacaatcaagcagaagaggctcaaggtcacagcagcaactacaagacagtcaccagaagattcgtcacttcaatgaaggagacgtactagtgattcctcttggtgttccttactggacctataacactggcgatgaaccagttgttgccatcagtcctcttgacacctccaacttcaacaatcagcttgatcaaaaccccagagtattttaccttgctgggaacccagatatagagcatcccgagaccatgcaacaacagcagcagcagaagagtcatggtggacgcaagcaggggcaacaccgacagcaggaggaagaaggtggcagtgtgctcagtggcttcagcaaacatttcttagcacaatccttcaacaccaacgaggacacagctgagaaacttcggtctccagatgacgaaaggaagcagatcgtgacagtggagggaggcctcagcgttatcagccccaagtggcaagaacaagaagacgaagacgaagacgaagacgaagaatatggacggacgccctcttatcctccacgacgaccaagccatggaaagcatgaagatgacgaggacgaggacgaagaagaagatcaacctcgtcctgatcaccctccacagcgaccaagcaggcccgaacaacaagaaccacgtggaagaggatgtcagactagaaatggggttgaggaaaatatttgcaccatgaagcttcacgagaacattgctcgcccttcacgtgctgacttctacaacccaaaagctggtcgcattagcaccctcaacagtctcaccctcccagccctccgccaattcggactcagtgcccaatatgttgtcctctacaggaatggaatttactctccagattggaacttgaacgcgaacagtgtgacgatgactcgagggaaaggaagagttagagtggtgaactgccaagggaatgcagtgttcgacggtgagctaaggaggggacaattgctagtggtgccgcagaaccccgcggtggctgagcaagggggagaacaaggattggaatatgtagtgttcaagacacaccacaacgccgtgagcagctacattaaggatgtgtttagggtaatcccttcggaggttctttccaattcttacaaccttggccagagtcaagtgcgtcagctcaagtatcaaggaaactccggccctttggtcaacccataa

SEQ ID NO: 26 - protein, 516 aa, Glycine max
MGKPFFTLSLSSLCLLLLSSACFAITSSKFNECQLNNLNALEPDHRVESEGGLIETWNSQHPELQCAGVTVSKRTLNRNGSHLPSYLPYPQMIIVVQGKGAIGFAFPGCPETFEKPQQQSSRRGSRSQQQLQDSHQKIRHFNEGDVLVIPLGVPYWTYNTGDEPVVAISPLDTSNFNNQLDQNPRVFYLAGNPDIEHPETMQQQQQQKSHGGRKQGQHRQQEEEGGSVLSGFSKHFLAQSFNTNEDTAEKLRSPDDERKQIVTVEGGLSVISPKWQEQEDEDEDEDEEYGRTPSYPPRRPSHGKHEDDEDEDEEEDQPRPDHPPQRPSRPEQQEPRGRGCQTRNGVEENICTMKLHENIARPSRADFYNPKAGRISTLNSLTLPALRQFGLSAQYVVLYRNGIYSPDWNLNANSVTMTRGKGRVRVVNCQGNAVFDGELRRGQLLVVPQNPAVAEQGGEQGLEYVVFKTHHNAVSSYIKDVFRVIPSEVLSNSYNLGQSQVRQLKYQGNSGPLVNP

SEQ ID NO: 27 - DNA, 1446 bp, Glycine max
  CDS (1)..(1443): glycinin G3 subunit
atggctaagcttgttctttccctttgttttctgcttttcagtggctgctgcttcgctttcagtttcagagagcagccacagcaaaacgagtgccagatccaacgcctcaatgccctaaaaccggataaccgtatagagtcagaaggtggcttcattgagacatggaaccctaacaacaagccattccagtgtgccggtgttgccctctctcgctgcaccctcaaccgcaacgcccttcgcagaccttcctacaccaacgctccccaggagatctacatccaacaaggtagtggtatttttggcatgatattcccgggttgtcctagcacatttgaagagcctcaacaaaaaggacaaagcagcaggccccaagaccgtcaccagaagatctatcacttcagagagggtgatttgattgcagtgccaaccggttttgcatactggatgtacaacaatgaagacactcctgttgttgccgtttctcttattgacaccaacagcttccagaaccagctcgaccagatgcctaggagattctatcttgctgggaaccaagagcaagagtttctacagtatcagccacagaagcagcaaggaggtactcaaagccagaaaggaaagcgtcagcaagaagaagaaaacgaaggaggcagcatattgagtggcttcgccccggaattcttggaacatgcgttcgtcgtggacaggcagatagtgagaaagctacaaggtgagaacgaagaggaagagaagggtgccattgtgacagtgaaaggaggtctcagcgtgataagcccacccacggaagagcagcaacaaagacccgaggaagaggagaagccagattgtgacgagaaagacaaacattgccaaagccaaagcagaaatggcattgacgagaccatttgcacaatgagacttcgccacaacattggccagacttcatcacctgacatcttcaaccctcaagctggtagcatcacaaccgctaccagcctcgacttcccagccctctcgtggctcaaactcagtgcccagtttggatcactccgcaagaatgctatgttcgtgccacactacaacctgaacgcaaacagcataatatacgcattgaatggacgggcattggtacaagtggtgaattgcaatggtgagagagtgtttgatggagagctgcaagagggacaggtgttaattgtgccacaaaactttgcggtggctgcaagatcacagagcgacaacttcgagtatgtttcattcaagaccaatgatagaccctcgatcggcaaccttgcaggtgcaaactcattgttgaacgcattgccggaggaagtgattcagcaaacttttaacctaaggaggcagcaggccaggcaggtcaagaacaacaaccctttcagcttcctggttccacctaaggagtctcagaggagagttgtggcttag

SEQ ID NO: 28 - protein, 481 aa, Glycine max
MAKLVLSLCFLLFSGCCFAFSFREQPQQNECQIQRLNALKPDNRIESEGGFIETWNPNNKPFQCAGVALSRCTLNRNALRRPSYTNAPQEIYIQQGSGIFGMIFPGCPSTFEEPQQKGQSSRPQDRHQKIYHFREGDLIAVPTGFAYWMYNNEDTPVVAVSLIDTNSFQNQLDQMPRRFYLAGNQEQEFLQYQPQKQQGGTQSQKGKRQQEEENEGGSILSGFAPEFLEHAFVVDRQIVRKLQGENEEEEKGAIVTVKGGLSVISPPTEEQQQRPEEEEKPDCDEKDKHCQSQSRNGIDETICTMRLRHNIGQTSSPDIFNPQAGSITTATSLDFPALSWLKLSAQFGSLRKNAMFVPHYNLNANSIIYALNGRALVQVVNCNGERVFDGELQEGQVLIVPQNFAVAARSQSDNFEYVSFKTNDRPSIGNLAGANSLLNALPEEVIQQTFNLRRQQARQVKNNNPFSFLVPPKESQRRVVA

SEQ ID NO: 29 - DNA, 1482 bp, Helianthus annuus
  CDS (1)..(1479): 11S storage protein G3-D1
atggcatccaaagcaactttgctcttagcttttacccttctctttgccacttgcattgcccgccaccagcaacggcaacagcaacagaaccagtgccagcttcaaaacatcgaggcgctcgagcccatcgaagttatccaagctgaagccggtgtgaccgaaatttgggacgcctatgaccaacagttccagtgtgcgtggtcgattttattcgacaccggattcaacctggtggccttctcttgccttcctacgtcaacacccctattttggccttcgtcgagagagggggttatattgccgggatgccgcagaacctatgaatattcgcaggagcaacagttttccggtgagggtggccgcagaggaggaggagagggcacattcaggaccgtcatcagaaagttagagaacttaaaggagggtgacgtggttgccatccccaccggaacagctcactggcttcacaacgacggcaacacagaacttgtggtcgtcttcttggatactcagaaccatgagaaccagcttgacgaaaaccaaaggagattcttcttagccggaaaccctcaagctcaagctcaaagccagcagcaacaacaaagacaaccacgccaacaatctcctcaaaggcaaaggcaaaggcaaaggcaagggcaaggtcagaacgccggcaacatcttcaacggtttcacccccgagctcattgcacaatcattcaacgtcgaccaagagaccgcccagaagctacaaggacaaaacgaccagagaggccacattgttaatgtcggacaagaccttcaaatagtccgcccaccacaagacagacgctctcctcgccaacaacaagagcaagcgacgtctcctcgccaacaacaagagcagcagcaaggcagacgtggcggatggagcaacggtgtggaagaaaccatctgcagcatgaagttcaaagtgaacattgacaacccttcccaggctgactttgtaaacccgcaagccggcagcattgcaaacctcaacagcttcaaattccccattctcgagcacctccggctcagcgtggaaagaggcgaactccgtccgaatgccatccaatccccacactggacaatcaacgcccacaatcttctctacgtaaccgagggagccttgagggtacaaatcgtcgacaaccaaggaaactcagttttcgacaacgagctccgtgagggacaggtggtggtgatcccgcagaactttgcggtgatcaagagagccaatgaacaaggaagcaggtgggtgtctttcaagactaatgataatgccatgatagcaaaccttgcagggcgtgtgtccgcatcagcagcatcgccgttgacgttgtgggcgaatcggtatcagctatctcgagaggaagctcagcagctcaagtttagccagagggagacggttttgtttgcaccaagtttttccaggggccaagggatcagggcttcacgttaa

SEQ ID NO: 30 - protein, 493 aa, Helianthus annuus
MASKATLLLAFTLLFATCIARHQQRQQQQNQCQLQNIEALEPIEVIQAEAGVTEIWDAYDQQFQCAWSILFDTGFNLVAFSCLPTSTPLFWPSSREGVILPGCRRTYEYSQEQQFSGEGGRRGGGEGTFRTVIRKLENLKEGDVVAIPTGTAHWLHNDGNTELVVVFLDTQNHENQLDENQRRFFLAGNPQAQAQSQQQQQRQPRQQSPQRQRQRQRQGQGQNAGNIFNGFTPELIAQSFNVDQETAQKLQGQNDQRGHIVNVGQDLQIVRPPQDRRSPRQQQEQATSPRQQQEQQQGRRGGWSNGVEETICSMKFKVNIDNPSQADFVNPQAGSIANLNSFKFPILEHLRLSVERGELRPNAIQSPHWTINAHNLLYVTEGALRVQIVDNQGNSVFDNELREGQVVVIPQNFAVIKRANEQGSRWVSFKTNDNAMIANLAGRVSASAASPLTLWANRYQLSREEAQQLKFSQRETVLFAPSFSRGQGIRASR

SEQ ID NO: 31 - DNA, 537 bp, Brassica napus
  CDS (1)..(534): NAPIN
atggcgaacaagctcttcctcgtctcggcaactctcgccttcttcttccttctcaccaatgcctccatctaccggacggtcgtcgagttcgacgaagatgatgccacagactcagccggcccatttaggattccaaaatgtaggaaggagtttcagcaagcacaacacctaagagcttgccagcagtggctccacaagcaagcaatgcagtctggcggtggtcctagctggaccctcgacggtgagtttgactttgaagacgacatggagaacccgcagggtccacagcagagaccgcctctactccagcagtgctgtaacgagctccaccaggaagagcccctttgcgtttgcccaaccttgaaaggagcatccaaagcggttaaacaacaaattcaacaacagggacaacagcaaggaaagcagcaaatggtgagccgtatctaccagaccgctacgcacttacctaaagtttgcaacatcccgcaagttagcgtttgtcccttccagaagaccatgcctgggccctcctactag

SEQ ID NO: 32 - protein, 178 aa, Brassica napus
MANKLFLVSATLAFFFLLTNASIYRTVVEFDEDDATDSAGPFRIPKCRKEFQQAQHLRACQQWLHKQAMQSGGGPSWTLDGEFDFEDDMENPQGPQQRPPLLQQCCNELHQEEPLCVCPTLKGASKAVKQQIQQQGQQQGKQQMVSRIYQTATHLPKVCNIPQVSVCPFQKTMPGPSY

SEQ ID NO: 33 - DNA, 537 bp, Brassica juncea
  CDS (1)..(534): 2S storage protein
atggcgaacaagctcttcctcgtctcggcaactctcgccttcttcttccttctcaccaatgcctccatctaccggacggtcgtcgagttcgacgaagatgatgccacagactcagccggcccatttaggattccaaaatgtaggaaggagtttcagcaagcacaacacctaagagtctgccagcagtggctccacaagcaagcaatgcagtctggcggtggtctcagctggaccctcgacggtgagtttgactttgaagacgacatggagaactcgcagggtccacagcagagaccgcctctactccagcagtgctgtaacgagctccaccaggaagagcccctttgcgtttgcccaaccttgaaaggagcatccaaagcggttaaacaacaaattcaacaacagggacaacagcaaggaaagcagcaaatggtgagccgtatctaccagaccgctacgcacttacctaaagtttgcaacatcccgcaagttagcgtttgtcccttccagaagaccatgcctgggccctcctactag

SEQ ID NO: 34 - protein, 178 aa, Brassica juncea
MANKLFLVSATLAFFFLLTNASIYRTVVEFDEDDATDSAGPFRIPKCRKEFQQAQHLRVCQQWLHKQAMQSGGGLSWTLDGEFDFEDDMENSQGPQQRPPLLQQCCNELHQEEPLCVCPTLKGASKAVKQQIQQQGQQQGKQQMVSRIYQTATHLPKVCNIPQVSVCPFQKTMPGPSY

SEQ ID NO: 35 - DNA, 537 bp, Brassica oleracea
  CDS (1)..(534): 2S storage protein
atggcgaacaagctcttcctcgtctcggcaactctcgccttcttcttccttctcaccaatgcctccatctaccggacggtggtcgagttcgacgaagatgatgccacaaacccagccggcccatttaggatcccaaaatgtaggaaggagtttcagcaagcacaacacctaagagcttgccagcagtggctccacaagcaagcaatgcagtctggcggtggtcctagctggaccctcgacagtgagtttgactttgaagacgacatggagaacccgcagggtccacagcagagaccgcctctactcctgcaatgctgtaacgagctggaccaggaagagcccctttgcgtttgcccaaccttgaaaggagcatccaaagcggttaaacaacaaattcaacaacagggacaacagcaaggaaagcagcaaatggtgagccgtatctaccagaccgctacgcacttacctaaagtttgcaacatcccgcaagttagcgtttgtcccttccagaagaccatgcctgggccctcctactag

SEQ ID NO: 36 - protein, 178 aa, Brassica oleracea
MANKLFLVSATLAFFFLLTNASIYRTVVEFDEDDATNPAGPFRIPKCRKEFQQAQHLRACQQWLHKQAMQSGGGPSWTLDSEFDFEDDMENPQGPQQRPPLLLQCCNELDQEEPLCVCPTLKGASKAVKQQIQQQGQQQGKQQMVSRIYQTATHLPKVCNIPQVSVCPFQKTMPGPSY

SEQ ID NO: 37 - DNA, 543 bp, Brassica napus cv. Topas
  CDS (1)..(540): Napin
atggcgaacaagctcttcctcgtctcggcaactcttgccttcttcttccttctcaccaacgcctccatctaccgcaccatcgtggaagtcgacgaagatgatgccacaaacccagccggcccatttaggattccaaaatgtaggaaggagtttcagcaagcacaacacctgaaagcttgccaacaatggctccacaagcaggcaatgcagtccggtagtggcccaagctggaccctcgacggtgagtttgattttgaagatgacatggagaacccccagggcccacaacagaggccgccactactccagcagtgctgcaacgagctccaccaggaagagccactttgcgtttgcccaaccttgaaaggagcatccaaagccgttaaacaacaggttcgacaacagcaaggacagcagggacagcagctgcagcaagtaattagccgtatctaccagactgctacgcacttacctaaagtttgcaacatcccgcaagttagcgtttgtcccttccagaagaccatgcctggaccctcctactag

SEQ ID NO: 38 - protein, 180 aa, Brassica napus cv. Topas
MANKLFLVSATLAFFFLLTNASIYRTIVEVDEDDATNPAGPFRIPKCRKEFQQAQHLKACQQWLHKQAMQSGSGPSWTLDGEFDFEDDMENPQGPQQRPPLLQQCCNELHQEEPLCVCPTLKGASKAVKQQVRQQQGQQGQQLQQVISRIYQTATHLPKVCNIPQVSVCPFQKTMPGPSY

SEQ ID NO: 39 - DNA, 435 bp, Sinapis alba
  CDS (1)..(432): coding for partial sin1 storage protein
ccagccggcccatttgggattccaaaatgtaggaaggagtttcaacaagcacaacacctaagagcttgccagcaatggctccacaagcaggcaatgcagtctggtagtggtccaagctggaccctcgacgatgagtttgattttgaagatgacatggagaacccacagggaccacagcagaggccaccactactccagcagtgctgcaacgagctccaccaggaagagccactttgcgtttgcccaaccttgaaaggagcatccaaagccgttaaacagcaggttagacaacagctggagcagcagggacagcagggaccgcacctgcagcatgtaattagccgtatctaccagaccgctacgcacttacctagagtttgcaacattaggcaagttagcgtttgtcccttccagaagaccatgcctggaccctcc

SEQ ID NO: 40 - protein, 144 aa, Sinapis alba
PAGPFGIPKCRKEFQQAQHLRACQQWLHKQAMQSGSGPSWTLDDEFDFEDDMENPQGPQQRPPLLQQCCNELHQEEPLCVCPTLKGASKAVKQQVRQQLEQQGQQGPHLQHVISRIYQTATHLPRVCNIRQVSVCPFQKTMPGP

SEQ ID NO: 41 - DNA, 468 bp, Glycine max
  CDS (1)..(465): 2S albumins 1
atgaccaagcttacaattctcctcatcgctcttctcttcatcgcccacacctgctgcgcctccaaatggcaacagcaccagcaagagagctgccgcgagcagctcaaggggatcaacctcaacccctgtgagcacatcatggagaagatccaagctggccgccgcggcgaggacggcagcgacgaagatcacattctcatcaggaccatgccgggaagaatcaactacatcaggaagaaggaaggaaaagaagaagaagaagaaggacacatgcagaagtgctgcagcgaaatgagcgagctgaaaagccccatatgccagtgcaaagcgctacagaagataatggataaccagagcgagcaactggaggggaaggagaagaagcagatggagagagagctcatgaacttggctattaggtgcaggttgggacccatgatagggtgcgacttgtcctccgatgactga

SEQ ID NO: 42 - protein, 155 aa, Glycine max
MTKLTILLIALLFIAHTCCASKWQQHQQESCREQLKGINLNPCEHIMEKIQAGRRGEDGSDEDHILIRTMPGRINYIRKKEGKEEEEEGHMQKCCSEMSELKSPICQCKALQKIMDNQSEQLEGKEKKQMERELMNLAIRCRLGPMIGCDLSSDD

SEQ ID NO: 43 - DNA, 477 bp, Glycine max
  CDS (1)..(474): 2S albumins
atgaccaagttcacaatcctcctcatctctcttctcttctgcatcgcccacacttgcagcgcctccaaatggcagcaccagcaagatagctgccgcaagcagctccagggggtgaacctcacgccctgcgagaagcacatcatggagaagatccaaggccgcggcgatgacgatgatgatgatgacgacgacaatcacattctcaggaccatgcggggaagaatcaactacataaggaggaacgaaggaaaagacgaagacgaagaagaagaaggacacatgcagaagtgctgcacagaaatgagcgagctgagaagccccaaatgccagtgcaaagcgctgcagaagataatggagaaccagagcgaggaactggaggagaagcagaagaagaaaatggagaaggagctcattaacttggctactatgtgcaggtttggacccatgatccagtgcgacttgtcctccgatgactaa

SEQ ID NO: 44 - protein, 158 aa, Glycine max
MTKFTILLISLLFCIAHTCSASKWQHQQDSCRKQLQGVNLTPCEKHIMEKIQGRGDDDDDDDDDNHILRTMRGRINYIRRNEGKDEDEEEEGHMQKCCTEMSELRSPKCQCKALQKIMENQSEELEEKQKKKMEKELINLATMCRFGPMIQCDLSSDD

SEQ ID NO: 45 - DNA, 537 bp, Brassica nigra
  CDS (1)..(534): 2S storage protein
atggcgaacaagctcttcctcgtctcggcaactctcgccttcttcttcctgctcaccaatgcctccatctaccggacggtcgtcgagttcgacgaagatgatgacacaaaccaagccggaccatttaggattccaagatgtcgaaaggagtttcggcaagcacaacatctaagagcttgccagcaatggctccacaggcaggcaatgcagtccggtagtggtccaagctggaccctggacggtgagtttgactttgaagacgacatggagaaccaacagggcccacagcagaggccacctctactccagcaatgctgcaacgagctccaccaggaagaggcactttgtgtttgcccaaccttgaaaggagcatccaaagcggttagacaacaggttcgacaacagggacaccagcagcagatgcagcatgtaattagccgtatctaccagaccgctacgcacttacctagagtttgcaacatcccgcaagttagcgtttgtcccttccagaagaccatgcctgggccctcctactag

SEQ ID NO: 46 - protein, 178 aa, Brassica nigra
MANKLFLVSATLAFFFLLTNASIYRTVVEFDEDDDTNQAGPFRIPRCRKEFRQAQHLRACQQWLHRQAMQSGSGPSWTLDGEFDFEDDMENQQGPQQRPPLLQQCCNELHQEEALCVCPTLKGASKAVRQQVRQQGHQQQMQHVISRIYQTATHLPRVCNIPQVSVCPFQKTMPGPSY

SEQ ID NO: 47 - DNA, 435 bp, Sinapis alba
  CDS (1)..(432): sin5 storage protein
ccagccggcccatttgggattccaaaatgtaggaaggagtttcaacaagcacaacacctaagagcttgccagcaatggctccacaagcaggcaatgcagtctggtagtggtccaagctggaccctcgacgatgagtttgattttgaagacgacatggagaacccccagggaccacagcagaagccgccactactccagcaatgctgcaacgagcttcaccaggaggagccactttgcgtttgcccaactttgaaaggagcttccaaagccgttaaacaacaggttcgacaacagttggggcagcagggacagcagggaccgcaggtgcagcatgtaattagccgtatctaccagaccgctacgcacttacctaaagtttgcaacatcccccaagtaagcgtttgtcccttcaagaagaccatgcctggaccctcc

SEQ ID NO: 48 - protein, 144 aa, Sinapis alba
PAGPFGIPKCRKEFQQAQHLRACQQWLHKQAMQSGSGPSWTLDDEFDFEDDMENPQGPQQKPPLLQQCCNELHQEEPLCVCPTLKGASKAVKQQVRQQLGQQGQQGPQVQHVISRIYQTATHLPKVCNIPQVSVCPFKKTMPGP

SEQ ID NO: 49 - DNA, 888 bp, Helianthus annuus
  CDS (1)..(885): HaG5 2S albumins
atggcaaagcaaatagttctcgcactcgctttcgccgcccttgtagcctttgctaccgcccacacaaccataatcaccaccaccatcgaagacgagaacccgatctccggacaaaggcaagtgagccaacggatacagggacaaaggctgaaccagtgtcgcatgttcctccagcagggtcagaacattcctcgcgaattcgataaccctcagatggggcggcagcaggagcagcagctccagcagtgttgtcaagagctccaaaacatcgaagggcagtgccaatgtgaggcggtgaagcaggtgttccgagaagcccagcagcaagtacaacagcaacagggacggcagcttgtacccttccgcggttcgcagcagacccaacagttgaagcagaaggctcagattctccctaacgtatgcaaccttcaatcaagacgatgtgaaatcggaaccatcaccaccaccgtcaccgagagcaatatcgatatccccttccgtgacaggccctttggcactggatcacaacagtgcagagaaactgaaatccaacgacccgttggtgaatgccaaaggttcgtggagcagcaaatgcagcagtctccgaggtccactagaccataccaacagcggccaggacaacagcagcagcagcagagagggctccaacaacaatgctgcaacgagctacaaaacgtgaagagggagtgtcattgcgaggcaattcaagaagtggctaggagagtgatgaggcagccacagcagcagcagcagcaacgtcgtgggcagttcggtgggcaggagatggaaaccgcgaggagggtgattcagaatctgcccaaccagtgcgacttggaagtccagcaatgcacaacctgtacgggatga

SEQ ID NO: 50 - protein, 295 aa, Helianthus annuus
MAKQIVLALAFAALVAFATAHTTIITTTIEDENPISGQRQVSQRIQGQRLNQCRMFLQQGQNIPREFDNPQMGRQQEQQLQQCCQELQNIEGQCQCEAVKQVFREAQQQVQQQQGRQLVPFRGSQQTQQLKQKAQILPNVCNLQSRRCEIGTITTTVTESNIDIPFRDRPFGTGSQQCRETEIQRPVGECQRFVEQQMQQSPRSTRPYQQRPGQQQQQQRGLQQQCCNELQNVKRECHCEAIQEVARRVMRQPQQQQQQRRGQFGGQEMETARRVIQNLPNQCDLEVQQCTTCTG

SEQ ID NO: 51 - DNA, 973 bp, Helianthus annuus
  CDS (2)..(970): coding for partial 2S albumins
ggcaaagataacacttctcttgctcgccttagctgctcttgtagccttggctacagcccacacaaccatcatcaccaccaccatcgacgacgagaacccgatctccgaacaaaggcaatgttggcaacaggtacagggacaaaggttgaaccagtgtcgcatgttcctccagcaaggtcagagggggcagcaacaccaacagcaacagcatcagcagcaggagcagcagctcctccagcagtgttgtcaagagcttcaaaacatcgaaggacagtgccaatgtgaggcggtgaagcaggtggtccgagatgctcagcgacacgagcaacagcgaccgcgagtgcccttccagggttctcagcagtctcaacagttgaagcagagggctcagattctccctaacgtatgcaaccttcaatcaagacgatgcgaaatcgaaagcgtcaggagtgttgctgagagcaattttgaaatcccatttgatatgccgtttgatatcccttggccctttcgcccaagctcagagtcacagcaatgcagacagagtgaaatccaaaggcctgtgagtcagtgccaaaggtatgtggagcagcaaattcagtcctccaggccataccaacagagcccgtacgaccggaggcaacagagcccatacgaccggaggcaacagagcccatatgaacagaggcaaggaccatacgaacagaggccatacgaacagaggccataccaacagcgaggaggacgacagcaggagcagcaagggctccagcaatgctgcaacgagctccaaaacgtgaggagggagtgtcagtgcgaggcgattaaggaagtgggccaaagaatgaggcagcagcaacaacaacaacgtaggcagtatggtgggcagcagacacaaactgtggagagaattcttgagaatctgcctaaccaatgcgacctagatgtccagcaatgcaacatcccctactga

SEQ ID NO: 52 - protein, 323 aa, Helianthus annuus
AKITLLLLALAALVALATAHTTIITTTIDDENPISEQRQCWQQVQGQRLNQCRMFLQQGQRGQQHQQQQHQQQEQQLLQQCCQELQNIEGQCQCEAVKQVVRDAQRHEQQRPRVPFQGSQQSQQLKQRAQILPNVCNLQSRRCEIESVRSVAESNFEIPFDMPFDIPWPFRPSSESQQCRQSEIQRPVSQCQRYVEQQIQSSRPYQQSPYDRRQQSPYDRRQQSPYEQRQGPYEQRPYEQRPYQQRGGRQQEQQGLQQCCNELQNVRRECQCEAIKEVGQRMRQQQQQQRRQYGGQQTQTVERILENLPNQCDLDVQQCNIPY

SEQ ID NO: 53 - DNA, 1114 bp, Artificial sequence
  Description of the artificial sequence DNA construct coding for dsRNA
ggccgcgtgttccatttggccggaaacaaccagcagggaggctttggcggttcacagcaacaacaagaacagaaaaacttgtggagcgggttcgacgcacaggtcatagctcaagcattgaaaattgacgttcagttggctcagcagcttcagaaccaacaagacagcagaggaaacatcgttcgtgttaagggacctttccaggtcgtgaggccacctctaagacagccctacgagagcgaggagtggagacacccacgtagcccacagggcaacggccttgaggagactatctgcagcatgaggtcccacgagaacattgacgaccctgctcgtgctgacgtgtacaagcccagcctaggtcgcgtgaccagcgtcaacagctataccttgcccatcttggagtatgtcaggctcagtgccactcgtggcgttctccagggtggatccttctgtaacatttgacaaaacatgtgaacacgtcatccgtcatatagaacttccaattttaatatgttttgctaaagaaaaaaaaaaggaataaatatctatcaaattcatttttaaaacatttgtatacgttcttaaataatttaggatatgactaatttttctttttggtaaaaatgttaatatctatatttaatttattaagaaaaatgtacttacaccctggagaacgccacgagtggcactgagcctgacatactccaagatgggcaaggtatagctgttgacgctggtcacgcgacctaggctgggcttgtacacgtcagcacgagcagggtcgtcaatgttctcgtgggacctcatgctgcagatagtctcctcaaggccgttgccctgtgggctacgtgggtgtctccactcctcgctctcgtagggctgtcttagaggtggcctcacgacctggaaaggtcccttaacacgaacgatgtttcctctgctgtcttgttggttctgaagctgctgagccaactgaacgtcaattttcaatgcttgagctatgacctgtgcgtcgaacccgctccacaagtttttctgttcttgttgttgctgtgaaccgccaaagcctccctgctggttgtttccggccaaatggaacacgcggccg

SEQ ID NO: 54 - RNA, 1114 nt, Artificial sequence
  Description of the artificial sequence DNA construct coding for dsRNA
ggccgcguguuccauuuggccggaaacaaccagcagggaggcuuuggcgguucacagcaacaacaagaacagaaaaacuuguggagcggguucgacgcacaggucauagcucaagcauugaaaauugacguucaguuggcucagcagcuucagaaccaacaagacagcagaggaaacaucguucguguuaagggaccuuuccaggucgugaggccaccucuaagacagcccuacgagagcgaggaguggagacacccacguagcccacagggcaacggccuugaggagacuaucugcagcaugaggucccacgagaacauugacgacccugcucgugcugacguguacaagcccagccuaggucgcgugaccagcgucaacagcuauaccuugcccaucuuggaguaugucaggcucagugccacucguggcguucuccaggguggauccuucuguaacauuugacaaaacaugugaacacgucauccgucauauagaacuuccaauuuuaauauguuuugcuaaagaaaaaaaaaaggaauaaauaucuaucaaauucauuuuuaaaacauuuguauacguucuuaaauaauuuaggauaugacuaauuuuucuuuuugguaaaaauguuaauaucuauauuuaauuuauuaagaaaaauguacuuacacccuggagaacgccacgaguggcacugagccugacauacuccaagaugggcaagguauagcuguugacgcuggucacgcgaccuaggcugggcuuguacacgucagcacgagcagggucgucaauguucucgugggaccucaugcugcagauagucuccucaaggccguugcccugugggcuacgugggugucuccacuccucgcucucguagggcugucuuagagguggccucacgaccuggaaaggucccuuaacacgaacgauguuuccucugcugucuuguugguucugaagcugcugagccaacugaacgucaauuuucaaugcuugagcuaugaccugugcgucgaacccgcuccacaaguuuuucuguucuuguuguugcugugaaccgccaaagccucccugcugguuguuuccggccaaauggaacacgcggccg

SEQ ID NO: 55 - DNA, 1789 bp, Artificial sequence
  Description of the artificial sequence DNA construct coding for dsRNA
gcggccgcggatcctcagggtcttttcttgcccactttcttgaacgccggcaaactcacgtttgttgttcacggaaggggtctaatgggaagagttattccgggatgcgccgagacgttcatggagtcaccggtatttggagaaggtcaaggtcagggtcagagtcaagggttccgtgacatgcaccagaaagtagagcacctacggtgcggtgacaccattgcaacaccatctggtgtagctcaatggttctacaacaatggaaatgagcctctcattcttgttgcagccgcggatctcgccagcaaccagaaccagcttgaccgcaaccttagaccatttttgatagccggaaacaacccacaagggcaggaatggctacaaggccgaaagcaacagaagcaaaacaacatcttcaatggcttcgcacctgagatcttggctcaagccttcaagatcaatgtcgagacggctcagcagctccagaaccagcaagataaccgtggcaacatcgtcaaggtcaacggacctttcggcgtcattaggccacccttgagacgcggcgaaggcggccaacaaccacatgaaatagctaatggtttagaggagactttgtgcaccatgcgatgcactgaaaacctcgatgacccgtcggatgctgacgtgtacaagccatcactcggatacattagcacacttaacagctacaatcttcctatcctcagacttctccgccttagcgctcttcgtggctccatccgtaaaactcgaggtaagctcaacaaatctttagaaaattaattttatgtgacatatgcaataatttgatttggcaagataaactaatagattttgcgatttggagttttaaactctaaataatctaaatcgttttcaattggtttaaatatatatcttgcatttttaatcgtttttaattaaaaaatatatatatatatatatatcttgcatttttaatcgttttcaatttaaaaaatatcttgcacgcagaacgctgtcgagttttacggatggagccacgaagagcgctaaggcggagaagtctgaggataggaagattgtagctgttaagtgtgctaatgtatccgagtgatggcttgtacacgtcagcatccgacgggtcatcgaggttttcagtgcatcgcatggtgcacaaagtctcctctaaaccattagctatttcatgtggttgttggccgccttcgccgcgtctcaagggtggcctaatgacgccgaaaggtccgttgaccttgacgatgttgccacggttatcttgctggttctggagctgctgagccgtctcgacattgatcttgaaggcttgagccaagatctcaggtgcgaagccattgaagatgttgttttgcttctgttgctttcggccttgtagccattcctgcccttgtgggttgtttccggctatcaaaaatggtctaaggttgcggtcaagctggttctggttgctggcgagatccgcggctgcaacaagaatgagaggctcatttccattgttgtagaaccattgagctacaccagatggtgttgcaatggtgtcaccgcaccgtaggtgctctactttctggtgcatgtcacggaacccttgactctgaccctgaccttgaccttctccaaataccggtgactccatgaacgtctcggcgcatcccggaataactcttcccattagaccccttccgtgaacaacaaacgtgagtttgccggcgttcaagaaagtgggcaagaaaagaccctgaggatccgcggccgc

SEQ ID NO: 56 - RNA, 1789 nt, Artificial sequence
  Description of the artificial sequence DNA construct coding for dsRNA
gcggccgcggauccucagggucuuuucuugcccacuuucuugaacgccggcaaacucacguuuguuguucacggaaggggucuaaugggaagaguuauuccgggaugcgccgagacguucauggagucaccgguauuuggagaaggucaaggucagggucagagucaaggguuccgugacaugcaccagaaaguagagcaccuacggugcggugacaccauugcaacaccaucugguguagcucaaugguucuacaacaauggaaaugagccucucauucuuguugcagccgcggaucucgccagcaaccagaaccagcuugaccgcaaccuuagaccauuuuugauagccggaaacaacccacaagggcaggaauggcuacaaggccgaaagcaacagaagcaaaacaacaucuucaauggcuucgcaccugagaucuuggcucaagccuucaagaucaaugucgagacggcucagcagcuccagaaccagcaagauaaccguggcaacaucgucaaggucaacggaccuuucggcgucauuaggccacccuugagacgcggcgaaggcggccaacaaccacaugaaauagcuaaugguuuagaggagacuuugugcaccaugcgaugcacugaaaaccucgaugacccgucggaugcugacguguacaagccaucacucggauacauuagcacacuuaacagcuacaaucuuccuauccucagacuucuccgccuuagcgcucuucguggcuccauccguaaaacucgagguaagcucaacaaaucuuuagaaaauuaauuuuaugugacauaugcaauaauuugauuuggcaagauaaacuaauagauuuugcgauuuggaguuuuaaacucuaaauaaucuaaaucguuuucaauugguuuaaauauauaucuugcauuuuuaaucguuuuuaauuaaaaaauauauauauauauauauaucuugcauuuuuaaucguuuucaauuuaaaaaauaucuugcacgcagaacgcugucgaguuuuacggauggagccacgaagagcgcuaaggcggagaagucugaggauaggaagauuguagcuguuaagugugcuaauguauccgagugauggcuuguacacgucagcauccgacgggucaucgagguuuucagugcaucgcauggugcacaaagucuccucuaaaccauuagcuauuucaugugguuguuggccgccuucgccgcgucucaaggguggccuaaugacgccgaaagguccguugaccuugacgauguugccacgguuaucuugcugguucuggagcugcugagccgucucgacauugaucuugaaggcuugagccaagaucucaggugcgaagccauugaagauguuguuuugcuucuguugcuuucggccuuguagccauuccugcccuuguggguuguuuccggcuaucaaaaauggucuaagguugcggucaagcugguucugguugcuggcgagauccgcggcugcaacaagaaugagaggcucauuuccauuguuguagaaccauugagcuacaccagaugguguugcaauggugucaccgcaccguaggugcucuacuuucuggugcaugucacggaacccuugacucugacccugaccuugaccuucuccaaauaccggugacuccaugaacgucucggcgcaucccggaauaacucuucccauuagaccccuuccgugaacaacaaacgugaguuugccggcguucaagaaagugggcaagaaaagacccugaggauccgcggccgc

SEQ ID NO: 57 - DNA, 1273 bp, Artificial sequence
  Description of the artificial sequence DNA construct coding for dsRNA
gcggccgcggatccatggctaacaagctcttcctcgtctgcgcaactctcgccctctgcttcctcctcaccaacgcttccatctaccgcaccgttgtcgaattcgaagaagatgacgccagcaaccccgtaggtccaagacagagatgccagaaggagtttcagcaatcacaacacctaagagcttgccagagatggatgagcaagcaaatgaggcaaggacgtggtggtggtccttccctcgacgatgagttcgatttcgagggcccccagcagggataccagctactccagcagtgctgcaacgagcttcgccaggaagagccagtttgcgtttgccccaccttgaaacaagctgccagggcagttagcctccagggacagcacggaccattccaatccaggaaaatttaccagtcagctaagtacttgcctaacatttgcaagatccagcaagttggtgaatgtcccttccagaccaccatccctttcttccctccttactactagggtactcgaggtaagctcaacaaatctttagaaaattaattttatgtgacatatgcaataatttgatttggcaagataaactaatagattttgcgatttggagttttaaactctaaataatctaaatcgttttcaattggtttaaatatatatcttgcatttttaatcgtttttaattaaaaaatatatatatatatatatatcttgcatttttaatcgttttcaatttaaaaaatatcttgcacgcagaacgctgtcgactaccctagtagtaaggagggaagaaagggatggtggtctggaagggacattcaccaacttgctggatcttgcaaatgttaggcaagtacttagctgactggtaaattttcctggattggaatggtccgtgctgtccctggaggctaactgccctggcagcttgtttcaaggtggggcaaacgcaaactggctcttcctggcgaagctcgttgcagcactgctggagtagctggtatccctgctgggggccctcgaaatcgaactcatcgtcgagggaaggaccaccaccacgtccttgcctcatttgcttgctcatccatctctggcaagctcttaggtgttgtgattgctgaaactccttctggcatctctgtcttggacctacggggttgctggcgtcatcttcttcgaattcgacaacggtgcggtagatggaagcgttggtgaggaggaagcagagggcgagagttgcgcagacgaggaagagcttgttagccatggat

SEQ ID NO: 58 - RNA, 1273 nt, Artificial sequence
  Description of the artificial sequence DNA construct coding for dsRNA
gcggccgcggauccauggcuaacaagcucuuccucgucugcgcaacucucgcccucugcuuccuccucaccaacgcuuccaucuaccgcaccguugucgaauucgaagaagaugacgccagcaaccccguagguccaagacagagaugccagaaggaguuucagcaaucacaacaccuaagagcuugccagagauggaugagcaagcaaaugaggcaaggacgugguggugguccuucccucgacgaugaguucgauuucgagggcccccagcagggauaccagcuacuccagcagugcugcaacgagcuucgccaggaagagccaguuugcguuugccccaccuugaaacaagcugccagggcaguuagccuccagggacagcacggaccauuccaauccaggaaaauuuaccagucagcuaaguacuugccuaacauuugcaagauccagcaaguuggugaaugucccuuccagaccaccaucccuuucuucccuccuuacuacuaggguacucgagguaagcucaacaaaucuuuagaaaauuaauuuuaugugacauaugcaauaauuugauuuggcaagauaaacuaauagauuuugcgauuuggaguuuuaaacucuaaauaaucuaaaucguuuucaauugguuuaaauauauaucuugcauuuuuaaucguuuuuaauuaaaaaauauauauauauauauauaucuugcauuuuuaaucguuuucaauuuaaaaaauaucuugcacgcagaacgcugucgacuacccuaguaguaaggagggaagaaagggaugguggucuggaagggacauucaccaacuugcuggaucuugcaaauguuaggcaaguacuuagcugacugguaaauuuuccuggauuggaaugguccgugcugucccuggaggcuaacugcccuggcagcuuguuucaagguggggcaaacgcaaacuggcucuuccuggcgaagcucguugcagcacugcuggaguagcugguaucccugcugggggcccucgaaaucgaacucaucgucgagggaaggaccaccaccacguccuugccucauuugcuugcucauccaucucuggcaagcucuuagguguugugauugcugaaacuccuucuggcaucucugucuuggaccuacgggguugcuggcgucaucuucuucgaauucgacaacggugcgguagauggaagcguuggugaggaggaagcagagggcgagaguugcgcagacgaggaagagcuuguuagccauggau

SEQ ID NO: 59 - DNA, 1575 bp, Arabidopsis thaliana
  CDS (1)..(1572): 12S cruciferin
atggttaagctcagcaatctcctcgttgcaaccttcggggttctcctcgtccttaacggctgccttgcgaggcagtcacttggggttcctcctcagctacagaacgagtgtaacctcgacaacctagatgttctccaagccaccgaaactatcaagagtgaagccggtcagatcgagtactgggaccacaaccaccctcagctccgatgtgttggtgtttccgttgctcgttatgtaattgaacaaggcggtctttacttgcccaccttcttcacttccccaaaaatttcctacgtcgttcaaggaacgggtatcagcggaagagtggtccctggatgtgccgagaccttcatggactcgcagccgatgcaaggacaacaacaaggccaaccatggcaaggacgacagggacaacaaggccaaccatgggaaggacagggacaacagggacaacaaggaagacaaggccaaccatgggaaggacagggacaacagggacaacaaggacgacagggacaacaaggccaaccatgggaaggacagggacagcagggacaacaagggttccgtgacatgcaccagaaggtggaacatgtgagacgcggagacgtctttgccaacactccaggctctgcccactggatctacaactcaggagaacagccacttgtcatcatcgctcttctcgacatcgccaactaccaaaaccaactcgaccgcaaccctagagtgttccatttggccggaaacaaccagcagggaggctttggcggttcacagcaacaacaagaacagaaaaacttgtggagcgggttcgacgcacaggtcatagctcaagcattgaaaattgacgttcagttggctcagcagcttcagaaccaacaagacagcagaggaaacatcgttcgtgttaagggacctttccaggtcgtgaggccacctctaagacagccctacgagagcgaggagtggagacacccacgtagcccacagggcaacggccttgaggagactatctgcagcatgaggtcccacgagaacattgacgaccctgctcgtgctgacgtgtacaagcccagcctaggtcgcgtgaccagcgtcaacagctataccttgcccatcttggagtatgtcaggctcagtgccactcgtggcgttctccagggtaatgcgatggtgcttcctaaatacaacatgaacgctaacgagatcttgtactgcactggaggacaaggaaggatccaagtggtcaacgacaacggacagaacgtgttggaccaacaggtgcagaagggacagctcgtggtcatcccacaagggttcgcatacgttgtccagtcccacggaaacaagttcgagtggatctctttcaaaactaatgaaaacgcaatgatcagcactttggcgggtagaacctcgctcttgagggcattgccattggaggtcatatcaaatggtttccagatctctcccgaggaagctaggaagatcaagttcaacacacttgagaccactttgacccgcgctgccggtaggcaacaacaacagttgatcgaggagattgtcgaggcttaa

SEQ ID NO: 60 - protein, 524 aa, Arabidopsis thaliana
MVKLSNLLVATFGVLLVLNGCLARQSLGVPPQLQNECNLDNLDVLQATETIKSEAGQIEYWDHNHPQLRCVGVSVARYVIEQGGLYLPTFFTSPKISYVVQGTGISGRVVPGCAETFMDSQPMQGQQQGQPWQGRQGQQGQPWEGQGQQGQQGRQGQPWEGQGQQGQQGRQGQQGQPWEGQGQQGQQGFRDMHQKVEHVRRGDVFANTPGSAHWIYNSGEQPLVIIALLDIANYQNQLDRNPRVFHLAGNNQQGGFGGSQQQQEQKNLWSGFDAQVIAQALKIDVQLAQQLQNQQDSRGNIVRVKGPFQVVRPPLRQPYESEEWRHPRSPQGNGLEETICSMRSHENIDDPARADVYKPSLGRVTSVNSYTLPILEYVRLSATRGVLQGNAMVLPKYNMNANEILYCTGGQGRIQVVNDNGQNVLDQQVQKGQLVVIPQGFAYVVQSHGNKFEWISFKTNENAMISTLAGRTSLLRALPLEVISNGFQISPEEARKIKFNTLETTLTRAAGRQQQQLIEEIVEA

SEQ ID NO: 61 - DNA, 1419 bp, Arabidopsis thaliana
  CDS (1)..(1416): 12S Cra1 storage protein
atggctcgagtctcttctcttctttctttctgcttaacacttttgatccttttccatggctacgcggctcaacagggtcagcagggtcagcagtttccgaacgagtgccagctcgaccagctcaatgcgctcgagccgtcacacgtactgaagagcgaggctggtcgcatcgaggtgtgggaccaccacgctcctcagctccgttgctcaggtgtctcctttgcacgttacatcatcgagtctaagggtctctacttgccctctttctttaacaccgcgaagctctctttcgtggctaagggacgaggtcttatgggaaaagtgatccctggatgcgccgaaacattccaagactcatcagagttccaaccacgcttcgaaggtcaaggtcaaagccagaggttccgtgacatgcaccagaaagtggagcacattaggagcggtgataccattgccacaacacccggtgtagcacagtggttctacaacgacggacagcagccacttgtcatcgtcagcgtcttcgatctagccagtcaccagaaccagcttgaccgcaacccaaggccattttacttagccggaaacaacccacaaggtcaagtatggctacaaggacgagagcaacagccacagaagaacattttcaatggatttggacccgaggttattgctcaagctttgaagatcgatcttcagacagcacagcaacttcagaaccaagatgacaaccgtggaaacattgtccgagtccaaggaccgttcggtgtcattaggccgcctttgaggggccagagacctcaggaggaggaagaagaagaaggacgacatggacgacacggtaatggcttagaggagaccatctgcagcgccaggtgcaccgataacctcgatgacccgtctcgtgctgacgtgtacaagccacagctcggttacatcagcactctcaacagttacgatctccccattcttcgcttcatccgtctctcagccctccgtggatctatccgtcaaaacgcaatggtgcttccacagtggaacgcaaacgcgaacgctattctttacgagacagacggggaagcccaaatccagatcgtaaacgacaatggtaacagagtgtttgacggacaagtctctcaaggacagctcatagccgtaccacaaggtttctcggtggtgaaacgcgcaacaagcaaccgattccagtgggttgagttcaaaacaaacgctaacgcgcaaatcaacactctggcgggacgaacctcagtcttgagaggtttaccacttgaagtcataaccaatgggttccaaatctcacccgaagaagcaaggagggtcaagttcaacacgctcgagaccactttgactcacagcagtggcccagctagctacggaaggccaagagtggctgcagcttaa

SEQ ID NO: 62 - protein, 472 aa, Arabidopsis thaliana
MARVSSLLSFCLTLLILFHGYAAQQGQQGQQFPNECQLDQLNALEPSHVLKSEAGRIEVWDHHAPQLRCSGVSFARYIIESKGLYLPSFFNTAKLSFVAKGRGLMGKVIPGCAETFQDSSEFQPRFEGQGQSQRFRDMHQKVEHIRSGDTIATTPGVAQWFYNDGQQPLVIVSVFDLASHQNQLDRNPRPFYLAGNNPQGQVWLQGREQQPQKNIFNGFGPEVIAQALKIDLQTAQQLQNQDDNRGNIVRVQGPFGVIRPPLRGQRPQEEEEEEGRHGRHGNGLEETICSARCTDNLDDPSRADVYKPQLGYISTLNSYDLPILRFIRLSALRGSIRQNAMVLPQWNANANAILYETDGEAQIQIVNDNGNRVFDGQVSQGQLIAVPQGFSVVKRATSNRFQWVEFKTNANAQINTLAGRTSVLRGLPLEVITNGFQISPEEARRVKFNTLETTLTHSSGPASYGRPRVAAA

SEQ ID NO: 63 - DNA, 1419 bp, Arabidopsis thaliana
  CDS (1)..(1416): At5g442120/MLN1_4 storage protein
atggctcgagtctcttctcttctttctttctgcttaacacttttgatccttttccatggctacgcggctcaacagggtcagcagggtcagcagtttccgaacgagtgccagctcgaccagctcaatgcgctcgagccgtcacacgtactgaagagcgaggctggtcgcatcgaggtgtgggaccaccacgctcctcagctccgttgctcaggtgtctcctttgcacgttacatcatcgagtctaagggtctctacttgccctctttctttaacaccgcgaagctctctttcgtggctaagggacgaggtcttatgggaaaagtgatccctggatgcgccgaaacattccaagactcatcagagttccaaccacgcttcgaaggtcaaggtcaaagccagaggttccgtgacatgcaccagaaagtggagcacattaggagcggtgataccattgccacaacacccggtgtagcacagtggttctacaacgacggacaggaaccacttgtcatcgtcagcgtcttcgatctagccagtcaccagaaccagcttgaccgcaacccaaggccattttacttagccggaaacaacccacaaggtcaagtatggctacaaggacgagagcaacagccacagaagaacattttcaatggatttggacccgaggttattgctcaagctttgaagatcgatcttcagacagcacagcaacttcagaaccaagatgacaaccgtggaaacattgtccgagtccaaggaccgttcggtgtcattaggccgcctttgaggggccagagacctcaggaggaggaagaagaagaaggacgacatggacgacacggtaatggcttagaggagaccatctgcagcgccaggtgcaccgataacctcgatgacccgtctcgtgctgacgtgtacaagccacagctcggttacatcagcactctcaacagttacgatctccccatccttcgcttcatccgtctctcagccctccgtggatctatccgtcaaaacgcaatggtgcttccacagtggaacgcaaacgcgaacgctattctttacgtgacagacggggaagcccaaatccagatcgtaaacgacaatggtaacagagtgtttgacggacaagtctctcaaggacagctcatagccgtaccacaaggtttctcggtggtgaaacgcgcaacaagcaaccgattccagtgggttgagttcaaaacaaacgctaacgcgcaaatcaacactctggcgggacgaacctcagtcttgagaggtttaccacttgaagtcataaccaatgggttccaaatctcacccgaagaagcaaggagggtcaagttcaacacgctcgagaccactttgactcacagcagtggcccagctagctacggaaggccaagggtggctgcagcttaa

SEQ ID NO: 64 - protein, 472 aa, Arabidopsis thaliana
MARVSSLLSFCLTLLILFHGYAAQQGQQGQQFPNECQLDQLNALEPSHVLKSEAGRIEVWDHHAPQLRCSGVSFARYIIESKGLYLPSFFNTAKLSFVAKGRGLMGKVIPGCAETFQDSSEFQPRFEGQGQSQRFRDMHQKVEHIRSGDTIATTPGVAQWFYNDGQEPLVIVSVFDLASHQNQLDRNPRPFYLAGNNPQGQVWLQGREQQPQKNIFNGFGPEVIAQALKIDLQTAQQLQNQDDNRGNIVRVQGPFGVIRPPLRGQRPQEEEEEEGRHGRHGNGLEETICSARCTDNLDDPSRADVYKPQLGYISTLNSYDLPILRFIRLSALRGSIRQNAMVLPQWNANANAILYVTDGEAQIQIVNDNGNRVFDGQVSQGQLIAVPQGFSVVKRATSNRFQWVEFKTNANAQINTLAGRTSVLRGLPLEVITNGFQISPEEARRVKFNTLETTLTHSSGPASYGRPRVAAA

SEQ ID NO: 65 - DNA, 1368 bp, Arabidopsis thaliana
  CDS (1)..(1365): 12S Crb storage protein
atgggtcgagtctcatctattatctctttctctttgacactcttgatcctcttcaatggctacactgcccaacagtggcccaacgagtgccagctcgatcaactcaatgcgctcgaaccatcccaaatcatcaagagcgagggtggtcgcatcgaggtctgggaccaccatgcaccccagctccgttgctccggctttgcctttgagcgtttcgtcattgagcctcagggtcttttcttgcccactttcttgaacgccggcaaactcacgtttgttgttcacggaaggggtctaatgggaagagttattccgggatgcgccgagacgttcatggagtcaccggtatttggagaaggtcaaggtcagggtcagagtcaagggttccgtgacatgcaccagaaagtagagcacctacggtgcggtgacaccattgcaacaccatctggtgtagctcaatggttctacaacaatggaaatgagcctctcattcttgttgcagccgcggatctcgccagcaaccagaaccagcttgaccgcaaccttagaccatttttgatagccggaaacaacccacaagggcaggaatggctacaaggccgaaagcaacagaagcaaaacaacatcttcaatggcttcgcacctgagatcttggctcaagccttcaagatcaatgtcgagacggctcagcagctccagaaccagcaagataaccgtggcaacatcgtcaaggtcaacggacctttcggcgtcattaggccacccttgagacgcggcgaaggcggccaacaaccacatgaaatagctaatggtttagaggagactttgtgcaccatgcgatgcactgaaaacctcgatgacccgtcggatgctgacgtgtacaagccatcactcggatacattagcacacttaacagctacaatcttcctatcctcagacttctccgccttagcgctcttcgtggctccatccgtaaaaacgctatggtgctaccgcaatggaacgtaaacgcaaacgcggcactctacgtgacaaacggaaaggctcatatacaaatggtgaacgacaacggagaaagagtgttcgaccaagagatctccagcggacagttactagtcgtgccacaaggcttttcggtcatgaaacatcgcataggcgaacagttcgagtggatcgaattcaagacaaacgaaaacgcacaggtcaacacactcgcgggccgtacctcagtcatgagaggtttgccgcttgaggttataaccaatgggtaccagatctctcccgaagaagctaaacgagtaaagtttagcacgattgagaccacactgacccatagcagtccaatgagctacggaaggcctagggcttga

SEQ ID NO: 66 - protein, 455 aa, Arabidopsis thaliana
MGRVSSIISFSLTLLILFNGYTAQQWPNECQLDQLNALEPSQIIKSEGGRIEVWDHHAPQLRCSGFAFERFVIEPQGLFLPTFLNAGKLTFVVHGRGLMGRVIPGCAETFMESPVFGEGQGQGQSQGFRDMHQKVEHLRCGDTIATPSGVAQWFYNNGNEPLILVAAADLASNQNQLDRNLRPFLIAGNNPQGQEWLQGRKQQKQNNIFNGFAPEILAQAFKINVETAQQLQNQQDNRGNIVKVNGPFGVIRPPLRRGEGGQQPHEIANGLEETLCTMRCTENLDDPSDADVYKPSLGYISTLNSYNLPILRLLRLSALRGSIRKNAMVLPQWNVNANAALYVTNGKAHIQMVNDNGERVFDQEISSGQLLVVPQGFSVMKHRIGEQFEWIEFKTNENAQVNTLAGRTSVMRGLPLEVITNGYQISPEEAKRVKFSTIETTLTHSSPMSYGRPRA

SEQ ID NO: 67 - DNA, 1356 bp, Arabidopsis thaliana
  CDS (1)..(1353): putative 12S storage protein
atgcataagcttttgttttctcttctctccgtcgtctcactctcatttctcctcttcttccatggcgccgaggcacgccagcgagaggcgccgtttccaaacgcctgccatttcagccaaatcaacagcctcgcgcccgctcaggcgacgaagttcgaagccggtcagatggaagtatgggaccacatgagccctgagctccgatgcgccggtgtaacggtggctcgcatcacccttcagcccaattccattttcttgcccgctttctttagcccacctgcccttgcttacgttgtccaaggagaaggagttatggggacgattgcttctggttgtcctgagacttttgcagaagttgaaggatcatcaggaagaggaggaggaggagacccgggtcgacgttttgaggacatgcaccagaagttggagaatttccggcgaggggatgtgtttgcttcgcttgccggagtttcacagtggtggtacaaccgcggtgattccgatgccgtcattgtcattgttcttgatgtcaccaacagagaaaaccagcttgaccaagtccctaggatgttccaactagccgggagcagaacgcaagaagaagaacaaccattaacgtggccatcaggcaacaacgctttcagcggtttcgacccaaacataatcgcggaagcattcaaaatcaacatcgagacagctaagcaactacaaaaccagaaggacaacagaggaaacataatccgagcaaatggtcctctccatttcgtcatcccaccgcctcgtgaatggcagcaagatggcattgctaatggcatcgaagagacttattgcacggctaagattcatgagaatatcgatgatccagaacggtctgaccattttagcacacgagccggaagaatcagcactcttaacagccttaatctccctgttctacgtctagtcagacttaacgcccttagaggttatctctacagcggaggaatggtgttgccacaatggacggcaaacgcgcacacggtgctatacgtcacaggaggtcaagccaagatacaagtggtggacgacaatggtcagtcggtgttcaatgagcaagtgggacaaggccaaatcattgtgattccacaaggctttgcagtttcaaaaacggctggtgaaacgggtttcgagtggatatcattcaagacaaacgataacgcttacattaacacactgagcggccaaacatcgtacttgagagcagttccagtggatgtgatcaaagcgtcatatggagtgaacgaggaagaagccaagaggatcaagtttagtcagcaagagaccatgttgtctatgacaccaagctcttcttcttaa

SEQ ID NO: 68 - protein, 451 aa, Arabidopsis thaliana
MHKLLFSLLSVVSLSFLLFFHGAEARQREAPFPNACHFSQINSLAPAQATKFEAGQMEVWDHMSPELRCAGVTVARITLQPNSIFLPAFFSPPALAYVVQGEGVMGTIASGCPETFAEVEGSSGRGGGGDPGRRFEDMHQKLENFRRGDVFASLAGVSQWWYNRGDSDAVIVIVLDVTNRENQLDQVPRMFQLAGSRTQEEEQPLTWPSGNNAFSGFDPNIIAEAFKINIETAKQLQNQKDNRGNIIRANGPLHFVIPPPREWQQDGIANGIEETYCTAKIHENIDDPERSDHFSTRAGRISTLNSLNLPVLRLVRLNALRGYLYSGGMVLPQWTANAHTVLYVTGGQAKIQVVDDNGQSVFNEQVGQGQIIVIPQGFAVSKTAGETGFEWISFKTNDNAYINTLSGQTSYLRAVPVDVIKASYGVNEEEAKRIKFSQQETMLSMTPSSSS

SEQ ID NO: 69 - DNA, 1356 bp, Arabidopsis thaliana; identical to SEQ ID NO: 67
  CDS (1)..(1353): 12S storage protein At1g03890

SEQ ID NO: 70 - protein, 451 aa, Arabidopsis thaliana; identical to SEQ ID NO: 68

SEQ ID NO: 71 - DNA, 867 bp, Arabidopsis thaliana
  CDS (1)..(864): prohibitin 1
atgaacaacgtcaaagttccaaagataccaggtggtggtgccatttcgacgttgcttaaggttgggattattggtgggcttggcctctatggtgctacgcacagtctctacaatgttgaaggaggacatcgagccatcatgttcaatcgtttagtcggtattaaagataaggtttaccctgagggtacacaccttatgattccttggtttgaaaggccggtcatctatgacgttcgtgctcgaccttaccttgttgagagtacatccggaagccgtgatcttcagatggtgaaaattgggcttagggttctcacacgtcccatggcagaccagttacctgaaatctacagaagccttggtgagaactacagcgagagagttctaccttctataatcaacgagactttgaaagctgtggttgctcagtacaatgcaagccagcttattactcagagagaggcggtcagtagggagatcaggaagattctgactgaacgagcagcaaacttcaatgttgcgcttgacgatgtgtccatcacaaacctgacattcgggaaggagttcacagctgccattgaagcaaagcaggtggcggctcaagaggctgagcgggctaagttcattgttgagaaggccgaacaagacaagagaagtgctgttatccgcgcccagggagaagccaagagtgctcagctcattggtcaagcaattgcaaacaaccaagcgtttatcacgctcaggaagatcgaggctgcaagagagattgcacagaccatagcaaactcggcgaacaaggtttacttgagctcagacgatcttttgcttaacctacaagggatgaatttggatgttgatgcaaagaactag

SEQ ID NO: 72 - protein, 288 aa, Arabidopsis thaliana
MNNVKVPKIPGGGAISTLLKVGIIGGLGLYGATHSLYNVEGGHRAIMFNRLVGIKDKVYPEGTHLMIPWFERPVIYDVRARPYLVESTSGSRDLQMVKIGLRVLTRPMADQLPEIYRSLGENYSERVLPSIINETLKAVVAQYNASQLITQREAVSREIRKILTERAANFNVALDDVSITNLTFGKEFTAAIEAKQVAAQEAERAKFIVEKAEQDKRSAVIRAQGEAKSAQLIGQAIANNQAFITLRKIEAAREIAQTIANSANKVYLSSDDLLLNLQGMNLDVDAKN

SEQ ID NO: 73 - DNA, 40 bp, Artificial sequence
  Description of the artificial sequence oligonucleotide primer
ataagaatgcggccgcgtgttccatttggccggaaacaac

SEQ ID NO: 74 - DNA, 33 bp, Artificial sequence
  Description of the artificial sequence oligonucleotide primer
cccggatccttctgtaacatttgacaaaacatg

SEQ ID NO: 75 - DNA, 40 bp, Artificial sequence; identical to SEQ ID NO: 73
  Description of the artificial sequence oligonucleotide primer

SEQ ID NO: 76 - DNA, 44 bp, Artificial sequence
  Description of the artificial sequence oligonucleotide primer
ataagaatgcggccgcggatccaccctggagaacgccacgagtg

SEQ ID NO: 77 - DNA, 45 bp, Artificial sequence
  Description of the artificial sequence oligonucleotide primer
ataagaatgcggccgcggatccctcagggtcttttcttgcccact

SEQ ID NO: 78 - DNA, 30 bp, Artificial sequence
  Description of the artificial sequence oligonucleotide primer
ccgctcgagtttacggatggagccacgaag

SEQ ID NO: 79 - DNA, 30 bp, Artificial sequence
  Description of the artificial sequence oligonucleotide primer
ccgctcgaggtaagctcaacaaatctttag

SEQ ID NO: 80 - DNA, 31 bp, Artificial sequence
  Description of the artificial sequence oligonucleotide primer
acgcgtcgacgcgttctgcgtgcaagatatt

SEQ ID NO: 81 - DNA, 46 bp, Artificial sequence
  Description of the artificial sequence oligonucleotide primer
ataagaatgcggccgcggatccatggctaacaagctcttcctcgtc

SEQ ID NO: 82 - DNA, 45 bp, Artificial sequence
  Description of the artificial sequence oligonucleotide primer
ataagaatgcggccgcggatccctagtagtaaggagggaagaaag

SEQ ID NO: 83 - DNA, 4954 bp, Artificial sequence
  Description of the artificial sequence DNA construct coding for dsRNA for suppression of multiple storage proteins
agcttggtaccgagctcggatccactagtaacggccgccagtgtgctggaattcgcccttgcggccgcgtgttccatttggccggaaacaaccagcagggaggctttggcggttcacagcaacaacaagaacagaaaaacttgtggagcgggttcgacgcacaggtcatagctcaagcattgaaaattgacgttcagttggctcagcagcttcagaaccaacaagacagcagaggaaacatcgttcgtgttaagggacctttccaggtcgtgaggccacctctaagacagccctacgagagcgaggagtggagacacccacgtagcccacagggcaacggccttgaggagactatctgcagcatgaggtcccacgagaacattgacgaccctgctcgtgctgacgtgtacaagcccagcctaggtcgcgtgaccagcgtcaacagctataccttgcccatcttggagtatgtcaggctcagtgccactcgtggcgttctccagggtggatccttctgtaacatttgacaaaacatgtgaacacgtcatccgtcatatagaacttccaattttaatatgttttgctaaagaaaaaaaaaaggaataaatatctatcaaattcatttttaaaacatttgtatacgttcttaaataatttaggatatgactaatttttctttttggtaaaaatgttaatatctatatttaatttattaagaaaaatgtacttacaccctggagaacgccacgagtggcactgagcctgacatactccaagatgggcaaggtatagctgttgacgctggtcacgcgacctaggctgggcttgtacacgtcagcacgagcagggtcgtcaatgttctcgtgggacctcatgctgcagatagtctcctcaaggccgttgccctgtgggctacgtgggtgtctccactcctcgctctcgtagggctgtcttagaggtggcctcacgacctggaaaggtcccttaacacgaacgatgtttcctctgctgtcttgttggttctgaagctgctgagccaactgaacgtcaattttcaatgcttgagctatgacctgtgcgtcgaacccgctccacaagtttttctgttcttgttgttgctgtgaaccgccaaagcctccctgctggttgtttccggccaaatggaacacgcggccgcaagggcgaattctgcagatatccatcacactggcggccgctcgacgtaagctcaacaaatctttagaaaattaattttatgtgacatatgcaataatttgatttggcaagataaactaatagattttgcgatttggagttttaaactctaaataatctaaatcgttttcaattggtttaaatatatatcttgcatttttaatcgtttttaattaaaaaatatatatatatatatatatcttgcatttttaatcgttttcaatttaaaaaatatcttgcacgcagaacgctctcgagcggccgcggatcctcagggtcttttcttgcccactttcttgaacgccggcaaactcacgtttgttgttcacggaaggggtctaatgggaagagttattccgggatgcgccgagacgttcatggagtcaccggtatttggagaaggtcaaggtcagggtcagagtcaagggttccgtgacatgcaccagaaagtagagcacctacggtgcggtgacaccattgcaacaccatctggtgtagctcaatggttctacaacaatggaaatgagcctctcattcttgttgcagccgcggatctcgccagcaaccagaaccagcttgaccgcaaccttagaccatttttgatagccggaaacaacccacaagggcaggaatggctacaaggccgaaagcaacagaagcaaaacaacatcttcaatggcttcgcacctgagatcttggctcaagccttcaagatcaatgtcgagacggctcagcagctccagaaccagcaagataaccgtggcaacatcgtcaaggtcaacggacctttcggcgtcattaggccacccttgagacgcggcgaaggcggccaacaaccacatgaaatagctaatggtttagaggagactttgtgcaccatgcgatgcactgaaaacctcgatgacccgtcggatgctgacgtgtacaagccatcactcggatacattagcacacttaacagctacaatcttcctatcctcagacttctccgccttagcgctcttcgtggctccatccgtaaaactcgaggtaagctcaacaaatctttagaaaattaattttatgtgacatatgcaataatttgatttggcaagataaactaatagattttgcgatttggagttttaaactctaaataatctaaatcgttttcaattggtttaaatatatatcttgcatttttaatcgtttttaattaaaaaatatatatatatatatatatcttgcatttttaatcgttttcaatttaaaaaatatcttgcacgcagaacgctgtcgagttttacggatggagccacgaagagcgctaaggcggagaagtctgaggataggaagattgtagctgttaagtgtgctaatgtatccgagtgatggcttgtacacgtcagcatccgacgggtcatcgaggttttcagtgcatcgcatggtgcacaaagtctcctctaaaccattagctatttcatgtggttgttggccgccttcgccgcgtctcaagggtggcctaatgacgccgaaaggtccgttgaccttgacgatgttgccacggttatcttgctggttctggagctgctgagccgtctcgacattgatcttgaaggcttgagccaagatctcaggtgcgaagccattgaagatgttgttttgcttctgttgctttcggccttgtagccattcctgcccttgtgggttgtttccggctatcaaaaatggtctaaggttgcggtcaagctggttctggttgctggcgagatccgcggctgcaacaagaatgagaggctcatttccattgttgtagaaccattgagctacaccagatggtgttgcaatggtgtcaccgcaccgtaggtgctctactttctggtgcatgtcacggaacccttgactctgaccctgaccttgaccttctccaaataccggtgactccatgaacgtctcggcgcatcccggaataactcttcccattagaccccttccgtgaacaacaaacgtgagtttgccggcgttcaagaaagtgggcaagaaaagaccctgaggatccgcggccgcgcatgcatctagctcgaggtaagctcaacaaatctttagaaaattaattttatgtgacatatgcaataatttgatttggcaagataaactaatagattttgcgatttggagttttaaactctaaataatctaaatcgttttcaattggtttaaatatatatcttgcatttttaatcgtttttaattaaaaaatatatatatatatatatatcttgcatttttaatcgttttcaatttaaaaaatatcttgcacgcagaacgctagggccgcggccgcggatccatggctaacaagctcttcctcgtctgcgcaactctcgccctctgcttcctcctcaccaacgcttccatctaccgcaccgttgtcgaattcgaagaagatgacgccagcaaccccgtaggtccaagacagagatgccagaaggagtttcagcaatcacaacacctaagagcttgccagagatggatgagcaagcaaatgaggcaaggacgtggtggtggtccttccctcgacgatgagttcgatttcgagggcccccagcagggataccagctactccagcagtgctgcaacgagcttcgccaggaagagccagtttgcgtttgccccaccttgaaacaagctgccagggcagttagcctccagggacagcacggaccattccaatccaggaaaatttaccagtcagctaagtacttgcctaacatttgcaagatccagcaagttggtgaatgtcccttccagaccaccatccctttcttccctccttactactagggtactcgaggtaagctcaacaaatctttagaaaattaattttatgtgacatatgcaataatttgatttggcaagataaactaatagattttgcgatttggagttttaaactctaaataatctaaatcgttttcaattggtttaaatatatatcttgcatttttaatcgtttttaattaaaaaatatatatatatatatatatcttgcatttttaatcgttttcaatttaaaaaatatcttgcacgcagaacgctgtcgactaccctagtagtaaggagggaagaaagggatggtggtctggaagggacattcaccaacttgctggatcttgcaaatgttaggcaagtacttagctgactggtaaattttcctggattggaatggtccgtgctgtccctggaggctaactgccctggcagcttgtttcaaggtggggcaaacgcaaactggctcttcctggcgaagctcgttgcagcactgctggagtagctggtatccctgctgggggccctcgaaatcgaactcatcgtcgagggaaggaccaccaccacgtccttgcctcatttgcttgctcatccatctctggcaagctcttaggtgttgtgattgctgaaactccttctggcatctctgtcttggacctacggggttgctggcgtcatcttcttcgaattcgacaacggtgcggtagatggaagcgttggtgaggaggaagcagagggcgagagttgcgcagacgaggaagagcttgttagccatggatcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgat

SEQ ID NO: 84 - RNA, 4954 nt, Artificial sequence
  Description of the artificial sequence  RNA coding for dsRNA for suppression of multiple storage proteins
agcuugguaccgagcucggauccacuaguaacggccgccagugugcuggaauucgcccuugcggccgcguguuccauuuggccggaaacaaccagcagggaggcuuuggcgguucacagcaacaacaagaacagaaaaacuuguggagcggguucgacgcacaggucauagcucaagcauugaaaauugacguucaguuggcucagcagcuucagaaccaacaagacagcagaggaaacaucguucguguuaagggaccuuuccaggucgugaggccaccucuaagacagcccuacgagagcgaggaguggagacacccacguagcccacagggcaacggccuugaggagacuaucugcagcaugaggucccacgagaacauugacgacccugcucgugcugacguguacaagcccagccuaggucgcgugaccagcgucaacagcuauaccuugcccaucuuggaguaugucaggcucagugccacucguggcguucuccaggguggauccuucuguaacauuugacaaaacaugugaacacgucauccgucauauagaacuuccaauuuuaauauguuuugcuaaagaaaaaaaaaaggaauaaauaucuaucaaauucauuuuuaaaacauuuguauacguucuuaaauaauuuaggauaugacuaauuuuucuuuuugguaaaaauguuaauaucuauauuuaauuuauuaagaaaaauguacuuacacccuggagaacgccacgaguggcacugagccugacauacuccaagaugggcaagguauagcuguugacgcuggucacgcgaccuaggcugggcuuguacacgucagcacgagcagggucgucaauguucucgugggaccucaugcugcagauagucuccucaaggccguugcccugugggcuacgugggugucuccacuccucgcucucguagggcugucuuagagguggccucacgaccuggaaaggucccuuaacacgaacgauguuuccucugcugucuuguugguucugaagcugcugagccaacugaacgucaauuuucaaugcuugagcuaugaccugugcgucgaacccgcuccacaaguuuuucuguucuuguuguugcugugaaccgccaaagccucccugcugguuguuuccggccaaauggaacacgcggccgcaagggcgaauucugcagauauccaucacacuggcggccgcucgacguaagcucaacaaaucuuuagaaaauuaauuuuaugugacauaugcaauaauuugauuuggcaagauaaacuaauagauuuugcgauuuggaguuuuaaacucuaaauaaucuaaaucguuuucaauugguuuaaauauauaucuugcauuuuuaaucguuuuuaauuaaaaaauauauauauauauauauaucuugcauuuuuaaucguuuucaauuuaaaaaauaucuugcacgcagaacgcucucgagcggccgcggauccucagggucuuuucuugcccacuuucuugaacgccggcaaacucacguuuguuguucacggaaggggucuaaugggaagaguuauuccgggaugcgccgagacguucauggagucaccgguauuuggagaaggucaaggucagggucagagucaaggguuccgugacaugcaccagaaaguagagcaccuacggugcggugacaccauugcaacaccaucugguguagcucaaugguucuacaacaauggaaaugagccucucauucuuguugcagccgcggaucucgccagcaaccagaaccagcuugaccgcaaccuuagaccauuuuugauagccggaaacaacccacaagggcaggaauggcuacaaggccgaaagcaacagaagcaaaacaacaucuucaauggcuucgcaccugagaucuuggcucaagccuucaagaucaaugucgagacggcucagcagcuccagaaccagcaagauaaccguggcaacaucgucaaggucaacggaccuuucggcgucauuaggccacccuugagacgcggcgaaggcggccaacaaccacaugaaauagcuaaugguuuagaggagacuuugugcaccaugcgaugcacugaaaaccucgaugacccgucggaugcugacguguacaagccaucacucggauacauuagcacacuuaacagcuacaaucuuccuauccucagacuucuccgccuuagcgcucuucguggcuccauccguaaaacucgagguaagcucaacaaaucuuuagaaaauuaauuuuaugugacauaugcaauaauuugauuuggcaagauaaacuaauagauuuugcgauuuggaguuuuaaacucuaaauaaucuaaaucguuuucaauugguuuaaauauauaucuugcauuuuuaaucguuuuuaauuaaaaaauauauauauauauauauaucuugcauuuuuaaucguuuucaauuuaaaaaauaucuugcacgcagaacgcugucgaguuuuacggauggagccacgaagagcgcuaaggcggagaagucugaggauaggaagauuguagcuguuaagugugcuaauguauccgagugauggcuuguacacgucagcauccgacgggucaucgagguuuucagugcaucgcauggugcacaaagucuccucuaaaccauuagcuauuucaugugguuguuggccgccuucgccgcgucucaaggguggccuaaugacgccgaaagguccguugaccuugacgauguugccacgguuaucuugcugguucuggagcugcugagccgucucgacauugaucuugaaggcuugagccaagaucucaggugcgaagccauugaagauguuguuuugcuucuguugcuuucggccuuguagccauuccugcccuuguggguuguuuccggcuaucaaaaauggucuaagguugcggucaagcugguucugguugcuggcgagauccgcggcugcaacaagaaugagaggcucauuuccauuguuguagaaccauugagcuacaccagaugguguugcaauggugucaccgcaccguaggugcucuacuuucuggugcaugucacggaacccuugacucugacccugaccuugaccuucuccaaauaccggugacuccaugaacgucucggcgcaucccggaauaacucuucccauuagaccccuuccgugaacaacaaacgugaguuugccggcguucaagaaagugggcaagaaaagacccugaggauccgcggccgcgcaugcaucuagcucgagguaagcucaacaaaucuuuagaaaauuaauuuuaugugacauaugcaauaauuugauuuggcaagauaaacuaauagauuuugcgauuuggaguuuuaaacucuaaauaaucuaaaucguuuucaauugguuuaaauauauaucuugcauuuuuaaucguuuuuaauuaaaaaauauauauauauauauauaucuugcauuuuuaaucguuuucaauuuaaaaaauaucuugcacgcagaacgcuagggccgcggccgcggauccauggcuaacaagcucuuccucgucugcgcaacucucgcccucugcuuccuccucaccaacgcuuccaucuaccgcaccguugucgaauucgaagaagaugacgccagcaaccccguagguccaagacagagaugccagaaggaguuucagcaaucacaacaccuaagagcuugccagagauggaugagcaagcaaaugaggcaaggacgugguggugguccuucccucgacgaugaguucgauuucgagggcccccagcagggauaccagcuacuccagcagugcugcaacgagcuucgccaggaagagccaguuugcguuugccccaccuugaaacaagcugccagggcaguuagccuccagggacagcacggaccauuccaauccaggaaaauuuaccagucagcuaaguacuugccuaacauuugcaagauccagcaaguuggugaaugucccuuccagaccaccaucccuuucuucccuccuuacuacuaggguacucgagguaagcucaacaaaucuuuagaaaauuaauuuuaugugacauaugcaauaauuugauuuggcaagauaaacuaauagauuuugcgauuuggaguuuuaaacucuaaauaaucuaaaucguuuucaauugguuuaaauauauaucuugcauuuuuaaucguuuuuaauuaaaaaauauauauauauauauauaucuugcauuuuuaaucguuuucaauuuaaaaaauaucuugcacgcagaacgcugucgacuacccuaguaguaaggagggaagaaagggaugguggucuggaagggacauucaccaacuugcuggaucuugcaaauguuaggcaaguacuuagcugacugguaaauuuuccuggauuggaaugguccgugcugucccuggaggcuaacugcccuggcagcuuguuucaagguggggcaaacgcaaacuggcucuuccuggcgaagcucguugcagcacugcuggaguagcugguaucccugcugggggcccucgaaaucgaacucaucgucgagggaaggaccaccaccacguccuugccucauuugcuugcucauccaucucuggcaagcucuuagguguugugauugcugaaacuccuucuggcaucucugucuuggaccuacgggguugcuggcgucaucuucuucgaauucgacaacggugcgguagauggaagcguuggugaggaggaagcagagggcgagaguugcgcagacgaggaagagcuuguuagccauggaucaauucgcccuauagugagucguauuacaauucacuggccgucguuuuacaacgucgugacugggaaaacccuggcguuacccaacuuaaucgccuugcagcacaucccccuuucgccagcuggcguaauagcgaagaggcccgcaccgau

SEQ ID NO: 85 - DNA, 4456 bp, Artificial sequence
  Description of the artificial sequence  DNA construct coding for dsRNA for suppression of multiple storage proteins
agcttggtaccgagctcggatccactagtaacggccgccagtgtgctggaattcgcccttgcggccgcggatcctcagggtcttttcttgcccactttcttgaacgccggcaaactcacgtttgttgttcacggaaggggtctaatgggaagagttattccgggatgcgccgagacgttcatggagtcaccggtatttggagaaggtcaaggtcagggtcagagtcaagggttccgtgacatgcaccagaaagtagagcacctacggtgcggtgacaccattgcaacaccatctggtgtagctcaatggttctacaacaatggaaatgagcctctcattcttgttgcagccgcggatctcgccagcaaccagaaccagcttgaccgcaaccttagaccatttttgatagccggaaacaacccacaagggcaggaatggctacaaggccgaaagcaacagaagcaaaacaacatcttcaatggcttcgcacctgagatcttggctcaagccttcaagatcaatgtcgagacggctcagcagctccagaaccagcaagataaccgtggcaacatcgtcaaggtcaacggacctttcggcgtcattaggccacccttgagacgcggcgaaggcggccaacaaccacatgaaatagctaatggtttagaggagactttgtgcaccatgcgatgcactgaaaacctcgatgacccgtcggatgctgacgtgtacaagccatcactcggatacattagcacacttaacagctacaatcttcctatcctcagacttctccgccttagcgctcttcgtggctccatccgtaaaaggatccgcggccgcaagggcgaattctgcagatccttcagggtcttttcttgcccactttcttgaacgccggcaaactcacgtttgttgttcacggaaggggtctaatgggaagagttattccgggatgcgccgagacgttcatggagtcaccggtatttggagaaggtcaaggtcagggtcagagtcaagggttccgtgacatgcaccagaaagtagagcacctacggtgcggtgacaccattgcaacaccatctggtgtagctcaatggttctacaacaatggaaatgagcctctcattcttgttgcagccgcggatctcgccagcaaccagaaccagcttgaccgcaaccttagaccatttttgatagccggaaacaacccacaagggcaggaatggctacaaggccgaaagcaacagaagcaaaacaacatcttcaatggcttcgcacctgagatcttggctcaagccttcaagatcaatgtcgagacggctcagcagctccagaaccagcaagataaccgtggcaacatcgtcaaggtcaacggacctttcggcgtcattaggccacccttgagacgcggcgaaggcggccaacaaccacatgaaatagctaatggtttagaggagactttgtgcaccatgcgatgcactgaaaacctcgatgacccgtcggatgctgacgtgtacaagccatcactcggatacattagcacacttaacagctacaatcttcctatcctcagacttctccgccttagcgctcttcgtggctccatccgtaaaagatcctatggctaacaagctcttcctcgtctgcgcaactctcgccctctgcttcctcctcaccaacgcttccatctaccgcaccgttgtcgaattcgaagaagatgacgccagcaaccccgtaggtccaagacagagatgccagaaggagtttcagcaatcacaacacctaagagcttgccagagatggatgagcaagcaaatgaggcaaggacgtggtggtggtccttccctcgacgatgagttcgatttcgagggcccccagcagggataccagctactccagcagtgctgcaacgagcttcgccaggaagagccagtttgcgtttgccccaccttgaaacaagctgccagggcagttagcctccagggacagcacggaccattccaatccaggaaaatttaccagtcagctaagtacttgcctaacatttgcaagatccagcaagttggtgaatgtcccttccagaccaccatccctttcttccctccttactactagggtagatatccatcacactggcggccgctcgacgtaagctcaacaaatctttagaaaattaattttatgtgacatatgcaataatttgatttggcaagataaactaatagattttgcgatttggagttttaaactctaaataatctaaatcgttttcaattggtttaaatatatatcttgcatttttaatcgtttttaattaaaaaatatatatatatatatatatcttgcatttttaatcgttttcaatttaaaaaatatcttgcacgcagaacgcctcgactaccctagtagtaaggagggaagaaagggatggtggtctggaagggacattcaccaacttgctggatcttgcaaatgttaggcaagtacttagctgactggtaaattttcctggattggaatggtccgtgctgtccctggaggctaactgccctggcagcttgtttcaaggtggggcaaacgcaaactggctcttcctggcgaagctcgttgcagcactgctggagtagctggtatccctgctgggggccctcgaaatcgaactcatcgtcgagggaaggaccaccaccacgtccttgcctcatttgcttgctcatccatctctggcaagctcttaggtgttgtgattgctgaaactccttctggcatctctgtcttggacctacggggttgctggcgtcatcttcttcgaattcgacaacggtgcggtagatggaagcgttggtgaggaggaagcagagggcgagagttgcgcagacgaggaagagcttgttagccataggatcttttacggatggagccacgaagagcgctaaggcggagaagtctgaggataggaagattgtagctgttaagtgtgctaatgtatccgagtgatggcttgtacacgtcagcatccgacgggtcatcgaggttttcagtgcatcgcatggtgcacaaagtctcctctaaaccattagctatttcatgtggttgttggccgccttcgccgcgtctcaagggtggcctaatgacgccgaaaggtccgttgaccttgacgatgttgccacggttatcttgctggttctggagctgctgagccgtctcgacattgatcttgaaggcttgagccaagatctcaggtgcgaagccattgaagatgttgttttgcttctgttgctttcggccttgtagccattcctgcccttgtgggttgtttccggctatcaaaaatggtctaaggttgcggtcaagctggttctggttgctggcgagatccgcggctgcaacaagaatgagaggctcatttccattgttgtagaaccattgagctacaccagatggtgttgcaatggtgtcaccgcaccgtaggtgctctactttctggtgcatgtcacggaacccttgactctgaccctgaccttgaccttctccaaataccggtgactccatgaacgtctcggcgcatcccggaataactcttcccattagaccccttccgtgaacaacaaacgtgagtttgccggcgttcaagaaagtgggcaagaaaagaccctgaaggatctgcagaattcgcccttgcggccgcggatccttttacggatggagccacgaagagcgctaaggcggagaagtctgaggataggaagattgtagctgttaagtgtgctaatgtatccgagtgatggcttgtacacgtcagcatccgacgggtcatcgaggttttcagtgcatcgcatggtgcacaaagtctcctctaaaccattagctatttcatgtggttgttggccgccttcgccgcgtctcaagggtggcctaatgacgccgaaaggtccgttgaccttgacgatgttgccacggttatcttgctggttctggagctgctgagccgtctcgacattgatcttgaaggcttgagccaagatctcaggtgcgaagccattgaagatgttgttttgcttctgttgctttcggccttgtagccattcctgcccttgtgggttgtttccggctatcaaaaatggtctaaggttgcggtcaagctggttctggttgctggcgagatccgcggctgcaacaagaatgagaggctcatttccattgttgtagaaccattgagctacaccagatggtgttgcaatggtgtcaccgcaccgtaggtgctctactttctggtgcatgtcacggaacccttgactctgaccctgaccttgaccttctccaaataccggtgactccatgaacgtctcggcgcatcccggaataactcttcccattagaccccttccgtgaacaacaaacgtgagtttgccggcgttcaagaaagtgggcaagaaaagaccctgactcgagcatgcat

SEQ ID NO: 86 - RNA, 4456 nt, Artificial sequence
  Description of the artificial sequence  RNA coding for dsRNA for suppression of multiple storage proteins
agcuugguaccgagcucggauccacuaguaacggccgccagugugcuggaauucgcccuugcggccgcggauccucagggucuuuucuugcccacuuucuugaacgccggcaaacucacguuuguuguucacggaaggggucuaaugggaagaguuauuccgggaugcgccgagacguucauggagucaccgguauuuggagaaggucaaggucagggucagagucaaggguuccgugacaugcaccagaaaguagagcaccuacggugcggugacaccauugcaacaccaucugguguagcucaaugguucuacaacaauggaaaugagccucucauucuuguugcagccgcggaucucgccagcaaccagaaccagcuugaccgcaaccuuagaccauuuuugauagccggaaacaacccacaagggcaggaauggcuacaaggccgaaagcaacagaagcaaaacaacaucuucaauggcuucgcaccugagaucuuggcucaagccuucaagaucaaugucgagacggcucagcagcuccagaaccagcaagauaaccguggcaacaucgucaaggucaacggaccuuucggcgucauuaggccacccuugagacgcggcgaaggcggccaacaaccacaugaaauagcuaaugguuuagaggagacuuugugcaccaugcgaugcacugaaaaccucgaugacccgucggaugcugacguguacaagccaucacucggauacauuagcacacuuaacagcuacaaucuuccuauccucagacuucuccgccuuagcgcucuucguggcuccauccguaaaaggauccgcggccgcaagggcgaauucugcagauccuucagggucuuuucuugcccacuuucuugaacgccggcaaacucacguuuguuguucacggaaggggucuaaugggaagaguuauuccgggaugcgccgagacguucauggagucaccgguauuuggagaaggucaaggucagggucagagucaaggguuccgugacaugcaccagaaaguagagcaccuacggugcggugacaccauugcaacaccaucugguguagcucaaugguucuacaacaauggaaaugagccucucauucuuguugcagccgcggaucucgccagcaaccagaaccagcuugaccgcaaccuuagaccauuuuugauagccggaaacaacccacaagggcaggaauggcuacaaggccgaaagcaacagaagcaaaacaacaucuucaauggcuucgcaccugagaucuuggcucaagccuucaagaucaaugucgagacggcucagcagcuccagaaccagcaagauaaccguggcaacaucgucaaggucaacggaccuuucggcgucauuaggccacccuugagacgcggcgaaggcggccaacaaccacaugaaauagcuaaugguuuagaggagacuuugugcaccaugcgaugcacugaaaaccucgaugacccgucggaugcugacguguacaagccaucacucggauacauuagcacacuuaacagcuacaaucuuccuauccucagacuucuccgccuuagcgcucuucguggcuccauccguaaaagauccuauggcuaacaagcucuuccucgucugcgcaacucucgcccucugcuuccuccucaccaacgcuuccaucuaccgcaccguugucgaauucgaagaagaugacgccagcaaccccguagguccaagacagagaugccagaaggaguuucagcaaucacaacaccuaagagcuugccagagauggaugagcaagcaaaugaggcaaggacgugguggugguccuucccucgacgaugaguucgauuucgagggcccccagcagggauaccagcuacuccagcagugcugcaacgagcuucgccaggaagagccaguuugcguuugccccaccuugaaacaagcugccagggcaguuagccuccagggacagcacggaccauuccaauccaggaaaauuuaccagucagcuaaguacuugccuaacauuugcaagauccagcaaguuggugaaugucccuuccagaccaccaucccuuucuucccuccuuacuacuaggguagauauccaucacacuggcggccgcucgacguaagcucaacaaaucuuuagaaaauuaauuuuaugugacauaugcaauaauuugauuuggcaagauaaacuaauagauuuugcgauuuggaguuuuaaacucuaaauaaucuaaaucguuuucaauugguuuaaauauauaucuugcauuuuuaaucguuuuuaauuaaaaaauauauauauauauauauaucuugcauuuuuaaucguuuucaauuuaaaaaauaucuugcacgcagaacgccucgacuacccuaguaguaaggagggaagaaagggaugguggucuggaagggacauucaccaacuugcuggaucuugcaaauguuaggcaaguacuuagcugacugguaaauuuuccuggauuggaaugguccgugcugucccuggaggcuaacugcccuggcagcuuguuucaagguggggcaaacgcaaacuggcucuuccuggcgaagcucguugcagcacugcuggaguagcugguaucccugcugggggcccucgaaaucgaacucaucgucgagggaaggaccaccaccacguccuugccucauuugcuugcucauccaucucuggcaagcucuuagguguugugauugcugaaacuccuucuggcaucucugucuuggaccuacgggguugcuggcgucaucuucuucgaauucgacaacggugcgguagauggaagcguuggugaggaggaagcagagggcgagaguugcgcagacgaggaagagcuuguuagccauaggaucuuuuacggauggagccacgaagagcgcuaaggcggagaagucugaggauaggaagauuguagcuguuaagugugcuaauguauccgagugauggcuuguacacgucagcauccgacgggucaucgagguuuucagugcaucgcauggugcacaaagucuccucuaaaccauuagcuauuucaugugguuguuggccgccuucgccgcgucucaaggguggccuaaugacgccgaaagguccguugaccuugacgauguugccacgguuaucuugcugguucuggagcugcugagccgucucgacauugaucuugaaggcuugagccaagaucucaggugcgaagccauugaagauguuguuuugcuucuguugcuuucggccuuguagccauuccugcccuuguggguuguuuccggcuaucaaaaauggucuaagguugcggucaagcugguucugguugcuggcgagauccgcggcugcaacaagaaugagaggcucauuuccauuguuguagaaccauugagcuacaccagaugguguugcaauggugucaccgcaccguaggugcucuacuuucuggugcaugucacggaacccuugacucugacccugaccuugaccuucuccaaauaccggugacuccaugaacgucucggcgcaucccggaauaacucuucccauuagaccccuuccgugaacaacaaacgugaguuugccggcguucaagaaagugggcaagaaaagacccugaaggaucugcagaauucgcccuugcggccgcggauccuuuuacggauggagccacgaagagcgcuaaggcggagaagucugaggauaggaagauuguagcuguuaagugugcuaauguauccgagugauggcuuguacacgucagcauccgacgggucaucgagguuuucagugcaucgcauggugcacaaagucuccucuaaaccauuagcuauuucaugugguuguuggccgccuucgccgcgucucaaggguggccuaaugacgccgaaagguccguugaccuugacgauguugccacgguuaucuugcugguucuggagcugcugagccgucucgacauugaucuugaaggcuugagccaagaucucaggugcgaagccauugaagauguuguuuugcuucuguugcuuucggccuuguagccauuccugcccuuguggguuguuuccggcuaucaaaaauggucuaagguugcggucaagcugguucugguugcuggcgagauccgcggcugcaacaagaaugagaggcucauuuccauuguuguagaaccauugagcuacaccagaugguguugcaauggugucaccgcaccguaggugcucuacuuucuggugcaugucacggaacccuugacucugacccugaccuugaccuucuccaaauaccggugacuccaugaacgucucggcgcaucccggaauaacucuucccauuagaccccuuccgugaacaacaaacgugaguuugccggcguucaagaaagugggcaagaaaagacccugacucgagcaugcau

SEQ ID NO: 87 - DNA, 33 bp, Artificial sequence
  Description of the artificial sequence oligonucleotide primer
aaaaggcctgtgttccatttggccggaaacaac

SEQ ID NO: 88 - DNA, 31 bp, Artificial sequence
  Description of the artificial sequence oligonucleotide primer
aaagatatcaccctggagaacgccacgagtg

SEQ ID NO: 89 - DNA, 33 bp, Artificial sequence
  Description of the artificial sequence oligonucleotide primer
aaaaggcctatggctaacaagctcttcctcgtc

SEQ ID NO: 90 - DNA, 32 bp, Artificial sequence
  Description of the artificial sequence oligonucleotide primer
aaagatatcctagtagtaaggagggaagaaag

SEQ ID NO: 91 - DNA, 32 bp, Artificial sequence
  Description of the artificial sequence oligonucleotide primer
ccgctcgagctcagggtcttttcttgcccact

SEQ ID NO: 92 - DNA, 32 bp, Artificial sequence
  Description of the artificial sequence oligonucleotide primer
ccggtcgacctagtagtaaggagggaagaaag

SEQ ID NO: 93 - DNA, 1500 bp, Arabidopsis thaliana
  CDS (1)..(1497): globuline-like protein
atgactagatttgcggtattgccactctctgttcttcttctcgttctcttgttcctctgcactgagtcgttggctaagtcggaggagtctgaagaatacgacgtcgctgtgccttcatgttgcgggttttcgtctcctcttctgattaagaaagatcaatggaaaccaatcttcgagacgaagtttggacagatctcaaccgttcaaatcggcaatggatgcggtggaatgggaccttacaagatacattccataacgttggagccaaacacaattttgctccctcttcttcttcattccgacatggtcttcttcgttgactctggaagtgggattctgaattgggttgacgaggaagcgaagagtaccgagatcagactaggagacgtttacaggctacgtcccggttcggttttctacttacagagcaaaccggatccttgctttggtgcctattcgagtatcacagatctaatgtttggttttgatgagacaattctccagtcagcttttggggttcctgaggggattatagagctgatgaggaaccgtacgaagccaccattgatcgtgagtgagacgctttgcacaccaggtgtggccaacacttggcagctccaaccgcgattactaaagctctttgctggaagtgcagacttggtggataacaagaagaagaaagagaagaaagagaagaaggagaaggtgaagaaagcgaagacattcaatgttttcgaatctgaaccggatttcgagagcccctacggtcgtactataacgattaacaggaaggatctgaaagttttaaaaggctcaatggttggagtttccatggtgaatctcactcaaggatcgatgatgggaccgcactggaatccatgggcttgcgagatttcgattgtattgaaaggagcaggaatggttagggttcttaggtcttcgatatcatcaaacacatcatcagagtgtaagaacgtgaggtttaaagtagaggaaggagatattttcgcagttccacggttacatccaatggctcaaatgtctttcaacaatgactcgttagtgttcgttgggtttactacttcagctaagaataacgagccgcagttcttagccggggaggactcggctttgcggatgcttgaccggcaagtattggctgcatcgcttaatgtgagtagtgtgacgattgatggattgttgggagctcagaaggaagctgttatcttggaatgtcattcttgtgcggaaggagagatagagaagcttaaggtggagatagagaggaagaaaatagatgatgagaggaagaggagacatgatgaaaggaagaaagaagaagaagaggcgaagagagaagaggaagagaggaggaaacgtgaagaagaagaagagaagaagcggtggccacctcaacaaccaccacaagaagaagaacttagggaacggcaactaccgatggagaaagaatgggaaatggaaggtgaagaggagagttaa

SEQ ID NO: 94 - protein, 499 aa, Arabidopsis thaliana
MTRFAVLPLSVLLLVLLFLCTESLAKSEESEEYDVAVPSCCGFSSPLLIKKDQWKPIFETKFGQISTVQIGNGCGGMGPYKIHSITLEPNTILLPLLLHSDMVFFVDSGSGILNWVDEEAKSTEIRLGDVYRLRPGSVFYLQSKPDPCFGAYSSITDLMFGFDETILQSAFGVPEGIIELMRNRTKPPLIVSETLCTPGVANTWQLQPRLLKLFAGSADLVDNKKKKEKKEKKEKVKKAKTFNVFESEPDFESPYGRTITINRKDLKVLKGSMVGVSMVNLTQGSMMGPHWNPWACEISIVLKGAGMVRVLRSSISSNTSSECKNVRFKVEEGDIFAVPRLHPMAQMSFNNDSLVFVGFTTSAKNNEPQFLAGEDSALRMLDRQVLAASLNVSSVTIDGLLGAQKEAVILECHSCAEGEIEKLKVEIERKKIDDERKRRHDERKKEEEEAKREEEERRKREEEEEKKRWPPQQPPQEEELRERQLPMEKEWEMEGEEES

SEQ ID NO: 95 - DNA, 1284 bp, Glycine max
  CDS (1)..(1281): 7S seed globuline
atggcttccatcctccactactttttagccctctctctttcttgctcttttcttttcttcttatccgactcagtcacccctacaaaaccaataaaccttgttgttctacccgttcaaaatgatggttccacagggctccattcggccaacctccaaaaaagaacccctctaatgcaagtaccagtcctggtggacctcaatggaaatcacttgtgggttaactgtgagcagcagtactcatccaaaacgtaccaagcacccttctgccactccacccaatgctctagagccaacacccaccaatgcctcagttgccccgcggcatcaaggccagggtgccacaaaaacacgtgtggcctcatgtccactaatcccatcacccaacaaaccggtttaggtgaactaggagaagacgttcttgcaatccacgccacacaagggtcgacccaacaacttggcccattggtcacagtcccacaattcctcttttcttgtgcaccttccttccttgttcaaaagggtcttcctagaaacactcaaggtgtggctgggttaggccatgcaccaatttctcttccaaaccaactcgcttcccactttggcctacaacgccaattcaccacttgcctttctcgctaccctacttcaaagggtgctataatattcggggatgcacctaacaacatgcgacagtttcaaaaccaagatattttccacgatttggccttcaccccattaaccatcaccctgcagggagagtacaacgtgagagtcaactcaataagaatcaaccagcacagtgtgttcccactgaacaagatatcatccaccatcgtagggtcgacctctggaggaaccatgattagcacctcaactcctcacatggttctccagcaatccgtgtaccaggctttcactcaggtgtttgctcagcagctaccaaagcaagcacaggtgaaatctgtggcaccatttgggttatgcttcaactccaacaagatcaatgcatatcctagcgtggaccttgtgatggacaagcccaatggtcctgtttggagaatctctggtgaggacttgatggtgcaggcacaacctggggtcacgtgtttgggtgttatgaatggaggaatgcaacctagagctgaaattaccttaggggcacgtcagttggaagagaacctggtggtgttcgatcttgcaaggtcaagggttgggtttagcacctcatcactgcactcgcatggagtcaaatgtgctgacctcttcaactttgccaatgcatga

SEQ ID NO: 96 - protein, 427 aa, Glycine max
MASILHYFLALSLSCSFLFFLSDSVTPTKPINLVVLPVQNDGSTGLHSANLQKRTPLMQVPVLVDLNGNHLWVNCEQQYSSKTYQAPFCHSTQCSRANTHQCLSCPAASRPGCHKNTCGLMSTNPITQQTGLGELGEDVLAIHATQGSTQQLGPLVTVPQFLFSCAPSFLVQKGLPRNTQGVAGLGHAPISLPNQLASHFGLQRQFTTCLSRYPTSKGAIIFGDAPNNMRQFQNQDIFHDLAFTPLTITLQGEYNVRVNSIRINQHSVFPLNKISSTIVGSTSGGTMISTSTPHMVLQQSVYQAFTQVFAQQLPKQAQVKSVAPFGLCFNSNKINAYPSVDLVMDKPNGPVWRISGEDLMVQAQPGVTCLGVMNGGMQPRAEITLGARQLEENLVVFDLARSRVGFSTSSLHSHGVKCADLFNFANA

SEQ ID NO: 97 - DNA, 814 bp, Zea mays
  CDS (1)..(720): 19kd zein
atggcagccaagatttttgccctccttgccctccttgctctttcagcaaacgttgctaccgcgactattattccacaatgctcacaacaatacctctctccggtgacagccgcgagatttgaatacccaactatacaatcctacaggctacaacaggccatcgcagcaagcatcttacggtcgttagcattgactgtccaacaaccatatgccctattgcaacaaccatccttagtgaatctatatctccaaagaatcgtagcacaacaactacaacaacaattgcttccaacaatcaatgaagtagttgcagcgaaccttgatgcttacctccagcaacaacaatttcttccattcaatcaactagctggggtgaaccctgctgcttacttgcaggcacaacagctactaccattcaaccaacttgtcaggagccctgctgccttcttactgcagcaacagttgttgccattccatctacaagttgtggcaaacattgctgctttcttgcaacaacaacaattgctgccattttacccacaggttgtgggaaacattaacgccttcttgcaacagcaacagttgctgccattctacccacaggatgtggcaaacaatgtcgccttcttacaacaacaacaattgctgccatttagccaacttgctttgacgaatcctaccaccttattgcagcagcccaccattggtggtgccatcttctagattttttatgctttatactgtaataataaagttctcatactgatatgtgcaacttctcagtaataaaagattagagatctatattttatta

SEQ ID NO: 98 - protein, 240 aa, Zea mays
MAAKIFALLALLALSANVATATIIPQCSQQYLSPVTAARFEYPTIQSYRLQQAIAASILRSLALTVQQPYALLQQPSLVNLYLQRIVAQQLQQQLLPTINEVVAANLDAYLQQQQFLPFNQLAGVNPAAYLQAQQLLPFNQLVRSPAAFLLQQQLLPFHLQVVANIAAFLQQQQLLPFYPQVVGNINAFLQQQQLLPFYPQDVANNVAFLQQQQLLPFSQLALTNPTTLLQQPTIGGAIF

SEQ ID NO: 99 - DNA, 705 bp, Zea mays
  CDS (1)..(702): 19 kd zein B1
atggcagccaaaatattttgcctccttatgctccttggtctttctgcaagtgctgctacggcgaccattttcccacaatgctcacaagctcctatagcttcccttcttcccccgtacctctcaccagcggtgtcttcggtatgtgaaaacccaattcttcaaccctataggatccaacaggcaatcgcagctggcatcttacctttatcacccttgttcctccaacaatcatcagccctattacagcagttacctttggtgcatttattggcacaaaacatcagggcacaacaactacaacaacttgtgctagcaaaccttgctgcctactctcagcaacaacagtttcttccattcaaccaactagctgcattgaactctgcttcttatttgcaacaacaacaactaccattcagccagctatctgctgcctacccccagcaatttcttccattcaaccaactgacagctttgaactctcctgcttatttacagcagcaacaactactaccattcagccagctagctggtgtgagccctgctaccttcttgacacaaccacaattgttgccgttctaccagcacgctgcgcctaacgctggcaccctcttacaactgcaacaattgctgccattcaaccaacttgctttgacaaacccaacagcattctaccaacaacccatcattggtggtgccctcttttag

SEQ ID NO: 100 - protein, 234 aa, Zea mays
MAAKIFCLLMLLGLSASAATATIFPQCSQAPIASLLPPYLSPAVSSVCENPILQPYRIQQAIAAGILPLSPLFLQQSSALLQQLPLVHLLAQNIRAQQLQQLVLANLAAYSQQQQFLPFNQLAALNSASYLQQQQLPFSQLSAAYPQQFLPFNQLTALNSPAYLQQQQLLPFSQLAGVSPATFLTQPQLLPFYQHAAPNAGTLLQLQQLLPFNQLALTNPTAFYQQPIIGGALF

SEQ ID NO: 101 - DNA, 804 bp, Zea mays
  CDS (1)..(801): 19 kd zein B2
atggcagccaaaatattttgcctcattatgctccttggtctttctgcaagtgctgctacggcgagcattttcccgcaatgctcacaagctcctatagcttcccttcttcccccatacctctcaccagcgatgtcttcagtatgtgaaaatccaattcttctaccctacaggatccaacaggcaatcgcagcaggcatcttacctttatcacccttgttcctccaacaatcatcagccctattacagcagttacctttggtgcatttattggcacaaaacatcagggcacaacaactacaacaactcgtgctagcaaaccttgctgcctactctcagcaacagcagttacctttggtgcatttgttggcacaaaacatcagggcacaacaactacaacaactcgtgctagcaaaccttgctgcctactctcagcaacaacagtttctgccattcaaccaactagctgcattgaactctgctgcttatttgcagcaacaacaactactaccattcagccagctagctgctgcctacccccggcaatttcttccattcaaccaactggcagcattgaactctcatgcttatgtacaacaacaacaactactaccattcagccagctagctgctgtgagccctgctgccttcttgacacagcaacatttgttgccgttctacctgcacactgcgcctaacgttggcaccctcttacaactgcaacaattgctgccattcgaccaacttgctttgacaaacccagcagtgttctaccaacaacccatcattggtggtgccctcttttag

SEQ ID NO: 102 - protein, 267 aa, Zea mays
MAAKIFCLIMLLGLSASAATASIFPQCSQAPIASLLPPYLSPAMSSVCENPILLPYRIQQAIAAGILPLSPLFLQQSSALLQQLPLVHLLAQNIRAQQLQQLVLANLAAYSQQQQLPLVHLLAQNIRAQQLQQLVLANLAAYSQQQQFLPFNQLAALNSAAYLQQQQLLPFSQLAAAYPRQFLPFNQLAALNSHAYVQQQQLLPFSQLAAVSPAAFLTQQHLLPFYLHTAPNVGTLLQLQQLLPFDQLALTNPAVFYQQPIIGGALF

SEQ ID NO: 103 - DNA, 801 bp, Zea mays
  CDS (1)..(798): 22kd alpha-zein
atggctaccaagatattagccctccttgcgctcctttccctttcagtgagcgcaacaactgcattcattattccacaatgctcacttgctcctaatgccattattccacagttcctcccatcagttacatcaatgggcatcgaacaccctattgtgcaagcctataggctacaacaagcgcttgcggcgagcgtcttacaacaaccgtttgcccaattacaacaacaatccttggcacatctaaccatacaaaccatcgcaacacaactagagcaacagtttgtgcccgcattgagccaactagccgcggtgaaccctgtctcctacttgcaacagcaaatgcttgcatccaacccacttgctctggcgaacacagccgcataccagcaacaactacagttgcaacagtttctaccagctcttagtcaactagccagggtgaaccctgccacatacctgcaacagcaacaactgctttcatctagcccactcgctgtgggcaatgcggctacatacctgcaacagcagctgctacaacagatcgtaccggctcttagtcagctagttgtggcgaaccctactgcctacttacaacagcttcttccattcaaccaactagatgtggcaaactctgctgcgtacctacaacagcggcagcaactacttaatccacttgcagcggctaacccattggtcgccgccttcctgcaacagcaacaatttctgccatacaaccaaatctctttgatgaaccttgccttgtcaaggcagcaaccgatcgttggaggtgccatcttttag

SEQ ID NO: 104 - protein, 266 aa, Zea mays
MATKILALLALLSLSVSATTAFIIPQCSLAPNAIIPQFLPSVTSMGIEHPIVQAYRLQQALAASVLQQPFAQLQQQSLAHLTIQTIATQLEQQFVPALSQLAAVNPVSYLQQQMLASNPLALANTAAYQQQLQLQQFLPALSQLARVNPATYLQQQQLLSSSPLAVGNAATYLQQQLLQQIVPALSQLVVANPTAYLQQLLPFNQLDVANSAAYLQQRQQLLNPLAAANPLVAAFLQQQQFLPYNQISLMNLALSRQQPIVGGAIF

SEQ ID NO: 105 - DNA, 471 bp, Oryza sativa
  CDS (1)..(468): prolamin
atgaagatcattttcgtatttgctctccttgctattgttgcatgcaacgcttctgcacggtttgatgctcttagtcaaagttatagacaatatcaactacaatcgcatctcctgctacagcaacaagtgctcagcccatgcagtgagttcgtaaggcaacagcatagcatagtggcaacccccttctggcaaccagctacgtttcaattgataaacaaccaagtcatgcagcaacagtgttgccaacagctcaggctggtagcgcaacaatctcactaccaggccattagtagcgttcaggcgattgtgcagcaactacagctgcagcaggtcggtgttgtctactttgatcagactcaagctcaagctcaagctttgctggccttaaacttgccatccatatgtggtatctatcctaactactacattgctccgaggagcattcccaccgttggtggtgtctggtactga

SEQ ID NO: 106 - protein, 156 aa, Oryza sativa
MKIIFVFALLAIVACNASARFDALSQSYRQYQLQSHLLLQQQVLSPCSEFVRQQHSIVATPFWQPATFQLINNQVMQQQCCQQLRLVAQQSHYQAISSVQAIVQQLQLQQVGVVYFDQTQAQAQALLALNLPSICGIYPNYYIAPRSIPTVGGVWY

SEQ ID NO: 107 - DNA, 645 bp, Avena sativa
  CDS (1)..(642): avenin
atgaagatcttcttcttcttagctctccttgctctggtagtgagcgccacctttgcacaatatgcagaatctgacggtagttatgaggaagtggagggttctcatgatcgatgccaacaacatcagatgaagctggactcttgcagagagtacgtggcggagcggtgcacaacgatgagagattttccgatcacctggccatggaaatggtggaagggtggttgcgaggagctccgcaatgagtgctgccaactgttgggccagatgccatcggagtgtcgctgtgatgcgatttggagatcaatccagcgcgagcttggtggcttctttggaactcaacaaggtctgatagggaaaaggttgaagatagccaagagtttgcccacgcagtcaacatgggccctgagtgcaatatccccaaactccatggttagccacattgctggaaagagctccattcttcgtgccttgcccgtggatgtcctcgccaatgcataccgcatttccaggcaagaagcccgaaacctcaaaaacaacaggggacaagagtctggtgtattcactccaaaatttacccaaacgagcttccaaccttatccagagggcgaggatgagtcatctttgattaataaggcatcagagtaa

SEQ ID NO: 108 - protein, 214 aa, Avena sativa
MKIFFFLALLALVVSATFAQYAESDGSYEEVEGSHDRCQQHQMKLDSCREYVAERCTTMRDFPITWPWKWWKGGCEELRNECCQLLGQMPSECRCDAIWRSIQRELGGFFGTQQGLIGKRLKIAKSLPTQSTWALSAISPNSMVSHIAGKSSILRALPVDVLANAYRISRQEARNLKNNRGQESGVFTPKFTQTSFQPYPEGEDESSLINKASE

SEQ ID NO: 109 - DNA, 1044 bp, Hordeum vulgare
  CDS (1)..(1041): c-hordein
atgaagacgttcctcacctttgtcctccttgccatggcgatgagcatcgtcactaccgctaggcagctaaaccctagccaccaagagttgcaatcaccacaacaaccatttctgaaacaacaatcatatctgcaacaaccatatccacaacaaccatatctaccgcagcaaccattccccacaccccaacaatttttcccctatctaccacagcaaacatttcccccatcccaacaaccaaaccccctacaaccacaacaaccattccccctgcaaccccaaccaccacaacaaccttttcctcagccccaacaaccaaatccccagcaaccacaacaacctttcccccggcaaccacaacaaatagtaccccagcaaccacaacaaccattccctcagcaaccacaacaaccttttcctcagccccaacaaccattctcttggcaaccacaacaaccatttctccagcccctacaactatagcccctgcaagcacaacaaccattccccttgcaacctcaactaccatttccgcaaccccaacaaccaattggacagcaaccaaaacaaccactcctgcagcaaccacaacaaacaattccccagcaaccacaacaaccattccccctgcagccgcaacaaccattcccccaacaaccacaacaaccacttccccaacaaccccaacaaataatttcccagcaaccccaacaaccattccctctacaacctcaacaaccattcccccaaccccaaccattcccccaggagcaaccccaacaagcattccccctacaaccgcaacaaccattccccgaggaatcagaacaaataattacccaacaaccattccctctacaaccacaacaactgttcccccagcaaccacaacaaccacttccccagccccaacaaccattccgccaactaccaaaatatataattccccagcaacctcaacaaccattccttctgcaaccacaccaacctcagcaaccttatgcacaacaagacatctggagtgatatagccctcttgggctaa

SEQ ID NO: 110 - protein, 160 aa, Hordeum vulgare
MKTFLTFVLLAMAMSIVTTARQLNPSHQELQSPQQPFLKQQSYLQQPYPQQPYLPQQPFPTPQQFFPYLPQQTFPPSQQPNPLQPQQPFPLQPQPPQQPFPQPQQPNPQQPQQPFPRQPQQIVPQQPQQPFPQQPQQPFPQPQQPFSWQPQQPFLQPLQL

SEQ ID NO: 111 - protein, 186 aa, Hordeum vulgare
PLQAQQPFPLQPQLPFPQPQQPIGQQPKQPLLQQPQQTIPQQPQQPFPLQPQQPFPQQPQQPLPQQPQQIISQQPQQPFPLQPQQPFPQPQPFPQEQPQQAFPLQPQQPFPEESEQIITQQPFPLQPQQLFPQQPQQPLPQPQQPFRQLPKYIIPQQPQQPFLLQPHQPQQPYAQQDIWSDIALLG

SEQ ID NO: 112 - DNA, 924 bp, Triticum aestivum
  CDS (1)..(921): glutenin-1D1
atgaagaccttcctcgtctttgccctcctcgccgttgcggcgacaagtgcaattgcgcagatggagactagatgcatccctggtttggagagaccatggcagcagcaaccattaccaccacaacagacatttccacaacaaccactattttcacaacaacaacaacaacaactatttcctcaacaaccatcattttcgcagcaacaaccaccattttggcagcaacaaccaccattttctcagcaacaaccaattctaccacagcaaccaccattttcgcagcaacaacaactagttctaccgcaacaaccaccattttcacagcaacaacaaccagttttacctccacaacaatcaccttttccacaacaacaacaacaacaccaacagctggtgcaacaacaaatccctgttgttcagccatccattttgcagcagctaaacccatgcaaggtattcctccagcagcagtgcagccctgtggcaatgccacaacgtcttgctaggtcgcaaatgttgcagcagagcagttgccatgtgatgcaacaacaatgttgccagcagttgccgcaaatcccccagcaatcccgctatgaggcaatccgtgctatcatctactccatcatcctgcaagaacaacaacaggttcagggttccatccaatctcagcagcagcaaccccaacagttgggccaatgtgtttcccaaccccaacagcagtcgcagcagcaactcgggcaacaacctcaacaacaacaattggcacagggtacctttttgcagccacaccagatagctcagcttgaggtgatgacttccattgcgctccgtatcctgccaacgatgtgcagtgttaatgtgccgttgtacagaaccaccactagtgtgccattcggcgttggcaccggagttggtgcctactga

SEQ ID NO: 113 - protein, 307 aa, Triticum aestivum
MKTFLVFALLAVAATSAIAQMETRCIPGLERPWQQQPLPPQQTFPQQPLFSQQQQQQLFPQQPSFSQQQPPFWQQQPPFSQQQPILPQQPPFSQQQQLVLPQQPPFSQQQQPVLPPQQSPFPQQQQQHQQLVQQQIPVVQPSILQQLNPCKVFLQQQCSPVAMPQRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGVGAY

SEQ ID NO: 114 - DNA, 8482 bp, Artificial sequence
  Description of the artificial sequence binary expression vector
ttccatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctagtaggaaacagctatgaccatgattacgccaatcaccactttgtacaagaaagctgggtctagatgacggacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcaggaggccttctagactgcaggcggccgcccaccgcggtgggctggctatgaagaaattataatcgtgtaaaacttagtgagtgtgtatgaatgaaagtattgcaaaatcctcattatatagactacatgcataactagttgcatgtaaatttgtagttttcttcattattgcatcctccaagtggatgtcatggttttacacatggcttccatgcaaatcatttccaaaatatttttaaactttccacagggcatccatgcatgcacctcaaaacttgtgtgtggtaacattgttgtcttgaaaaattactaaaccttttgtccacgtgacgttcatgcacctcaaatcttgtgtggtaccattattatcctcaagaattattgaatgtttggtgtatatgccatccatgcagcattgcaacaattaaatctccaaaccttgtggtaccatattcactcactttaattctcctatagtagaaatattagcaaatatttacatttccagttgattagtatatgtatttagaagacaaaaataatttagaatcaattaatcaacttgcaaattgctaagtgttggcaaacgttagcataaaaggtgttataaatttagtaccaaatataaaaatttatcgcaaatcaaatacataacacacatagtaaaacaaaaacaaattacaagggtttagacgtttagtggcaatgtgtaaatttgctcgactgaattggttcctttaagcctgcttttttgtacaaacttgtgataattcactggccgtcgttttacaacgactcaggatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccaagctctagatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaagctctgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgttaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcg

SEQ ID NO: 115 - DNA, 575 bp, Brassica napus
  misc_feature (1)..(6): restriction site
gtcgacgggccgatgggggcgaagggtcttgctgcaccaagagattttcttgcaccaacggcatggtttgaggaagggctacggcctgactacactattgttcagaagtttggcggtgaactctttactgctaaacaagatttctctccgttcaatgtggttgcctggcatggcaattacgtgccttataagtatgacctgcacaagttctgtccatacaacactgtccttgtagaccatggagatccatctgtaaatacagttctgacagcaccaacggataaacctggtgtggccttgcttgattttgtcatattccctcctcgttggttggttgctgagcatacctttcgacctccttactaccatcgtaactgcatgagtgaatttatgggcctaatctatggtgcttacgaggccaaagctgatggatttctacctggtggcgcaagtcttcacagttgtatgacacctcatggtccagatacaaccacatacgaggcgacgattgctcgtgtaaatgcaatggctccttataagctcacaggcaccatggccttcatgtttgaggtacc

SEQ ID NO: 116 - DNA, 1386 bp, Arabidopsis thaliana
  CDS (1)..(1383): coding for homogentisate-1,2-dioxygenase (HDG)
atggaagagaagaagaaggagcttgaagagttgaagtatcaatcaggttttggtaaccacttctcatcggaagcaatcgccggagctttaccgttagatcagaacagtcctcttctttgtccttacggtctttacgccgaacagatctccggtacttctttcacttctcctcgcaagctcaatcaaagaagttggttgtaccgggttaaaccatcggttacacatgaaccgttcaagcctcgtgtaccagctcataagaagcttgtgagtgagtttgatgcatcaaatagtcgtacgaatccgactcagcttcggtggagacctgaggatattcctgattcggagattgatttcgttgatgggttatttaccatttgtggagctggaagctcgtttcttcgccatggcttcgctattcacatgtatgtggctaacacaggaatgaaagactccgcattttgcaacgctgatggtgacttcttgttagttcctcaaacaggaaggctatggattgaaactgagtgtggaaggcttttggtaactcctggtgagattgctgttataccacaaggtttccgtttctccatagatttaccggatgggaagtctcgtggttatgttgctgaaatctatggggctcattttcagcttcctgatcttggaccaataggtgctaatggtcttgctgcatcaagagattttcttgcaccaacagcatggtttgaggatggattgcggcctgaatacacaattgttcagaagtttggcggtgaactctttactgctaaacaagatttctctccattcaatgtggttgcctggcatggcaattacgtgccttataagtatgacctgaagaagttctgtccatacaacactgtgcttttagatcatggagatccatctataaatacagtccttacagcaccaactgataaacctggtgtggccttgcttgattttgtcatatttcctcctcgatggttggttgctgagcatacttttcgacctccttactatcatcgtaactgcatgagtgaatttatgggcttaatctacggtgcatacgaggcgaaagctgatggatttctccctggcggtgcaagtcttcatagctgtatgacacctcatggtccagatactaccacgtacgaggcgacaattgctcgagtaaatgcaatggctccttctaaactcacaggtacgatggctttcatgttcgaatcagcattgatccctagagtctgtcattgggctctggagtctcctttcctggatcacgactactaccagtgttggattggcctcaagtctcatttctcgcgcataagcttggacaagacaaatgttgaatcaacagagaaagaaccaggagcttcggagtaa

SEQ ID NO: 117 - protein, 461 aa, Arabidopsis thaliana
MEEKKKELEELKYQSGFGNHFSSEAIAGALPLDQNSPLLCPYGLYAEQISGTSFTSPRKLNQRSWLYRVKPSVTHEPFKPRVPAHKKLVSEFDASNSRTNPTQLRWRPEDIPDSEIDFVDGLFTICGAGSSFLRHGFAIHMYVANTGMKDSAFCNADGDFLLVPQTGRLWIETECGRLLVTPGEIAVIPQGFRFSIDLPDGKSRGYVAEIYGAHFQLPDLGPIGANGLAASRDFLAPTAWFEDGLRPEYTIVQKFGGELFTAKQDFSPFNVVAWHGNYVPYKYDLKKFCPYNTVLLDHGDPSINTVLTAPTDKPGVALLDFVIFPPRWLVAEHTFRPPYYHRNCMSEFMGLIYGAYEAKADGFLPGGASLHSCMTPHGPDTTTYEATIARVNAMAPSKLTGTMAFMFESALIPRVCHWALESPFLDHDYYQCWIGLKSHFSRISLDKTNVESTEKEPGASE

SEQ ID NO: 118 - DNA, 815 bp, Arabidopsis thaliana
  CDS (37)..(705): coding for maleylacetoacetate isomerase (MAAI)
gtaatctccgaagaagaacaaattccttgctgaatcatgtcttatgttaccgatttttatcaggcgaagttgaagctctactcttactggagaagctcatgtgctcatcgcgtccgtatcgccctcactttaaaagggcttgattatgaatatataccggttaatttgctcaaaggggatcaatccgattcagatttcaagaagatcaatccaatgggcactgtaccagcgcttgttgatggtgatgttgtgattaatgactctttcgcaataataatgtacctggatgataagtatccggagccaccgctgttaccaagtgactaccataaacgggcggtaaattaccaggcgacgagtattgtcatgtctggtatacagcctcatcaaaatatggctctttttaggtatctcgaggacaagataaatgctgaggagaaaactgcttggattactaatgctatcacaaaaggattcacagctctcgagaaactgttggtgagttgcgctggaaaatacgcgactggtgatgaagtttacttggctgatcttttcctagcaccacagatccacgcagcattcaacagattccatattaacatggaaccattcccgactcttgcaaggttttacgagtcatacaacgaactgcctgcatttcaaaatgcagtcccggagaagcaaccagatactccttccaccatctgattctgtgaaccgtaagcttctctcagtctcagctcaataaaatctcttaggaaacaacaacaacaccttgaacttaaatgtatcatatgaaccagtttacaaataat

SEQ ID NO: 119 - protein, 223 aa, Arabidopsis thaliana
MSYVTDFYQAKLKLYSYWRSSCAHRVRIALTLKGLDYEYIPVNLLKGDQSDSDFKKINPMGTVPALVDGDVVINDSFAIIMYLDDKYPEPPLLPSDYHKRAVNYQATSIVMSGIQPHQNMALFRYLEDKINAEEKTAWITNAITKGFTALEKLLVSCAGKYATGDEVYLADLFLAPQIHAAFNRFHINMEPFPTLARFYESYNELPAFQNAVPEKQPDTPSTI

SEQ ID NO: 120 - DNA, 1227 bp, Arabidopsis thaliana
  CDS (1)..(1224): coding for fumarylacetoacetate hydrolase (FAAH)
atggcgttgctgaagtctttcatcgatgttggctcagactcgcacttccctatccagaatctcccttatggtgtcttcaaaccggaatcgaactcaactcctcgtcctgccgtcgctatcggcgatttggttctggacctctccgctatctctgaagctgggcttttcgatggtctgatccttaaggacgcagattgctttcttcagcctaatttgaataagttcttggccatgggacggcctgcgtggaaggaagcgcgttctacgctgcaaagaatcttgtcatttttgttatttggcttcaaggttttggttttggtatgttttcatgcagctaatgaacctatcttgcgagacaatgatgttttgaggagaaaatcattccatcagatgagtaaagtggaaatgattgttcctatggtgattggggactatacagacttctttgcatctatgcatcacgcgaagaactgcggacttatgttccgtgggcctgagaatgcgataaacccaaattggtttcgtcttcccattgcatatcatggacgggcatcatctattgtcatctctgggactgacattattcgaccaagaggtcagggccatccacaaggaaactctgaaccatattttggaccttcgaagaaacttgattttgagcttgagatggctgctgtggttggtccaggaaatgaattgggaaagcctattgacgtgaataatgcagccgatcatatatttggtctattactgatgaatgactggagtgctagggatattcaggcgtgggagtatgtacctcttggtcctttcctggggaagagttttgggactactatatccccttggattgttaccttggatgcgcttgagccttttggttgtcaagctcccaagcaggatccacctccattgccatatttggctgagaaagagtctgtaaattacgatatctccttggagctagcacaccataccgttaacggttgcaatttgaggcctggtgatctccttggcacaggaaccataagcggaccggagccagattcatatgggtgcctacttgagttgacatggaatggacagaaacctctatcactcaatggaacaactcagacgtttctcgaagacggagaccaagtcaccttctcaggtgtatgcaagggagatggttacaatgttgggtttggaacatgcacagggaaaattgttccttcaccgccttga

SEQ ID NO: 121 - protein, 408 aa, Arabidopsis thaliana
MALLKSFIDVGSDSHFPIQNLPYGVFKPESNSTPRPAVAIGDLVLDLSAISEAGLFDGLILKDADCFLQPNLNKFLAMGRPAWKEARSTLQRILSFLLFGFKVLVLVCFHAANEPILRDNDVLRRKSFHQMSKVEMIVPMVIGDYTDFFASMHHAKNCGLMFRGPENAINPNWFRLPIAYHGRASSIVISGTDIIRPRGQGHPQGNSEPYFGPSKKLDFELEMAAVVGPGNELGKPIDVNNAADHIFGLLLMNDWSARDIQAWEYVPLGPFLGKSFGTTISPWIVTLDALEPFGCQAPKQDPPPLPYLAEKESVNYDISLELAHHTVNGCNLRPGDLLGTGTISGPEPDSYGCLLELTWNGQKPLSLNGTTQTFLEDGDQVTFSGVCKGDGYNVGFGTCTGKIVPSPP

SEQ ID NO: 122 - DNA, 11667 bp, Artificial sequence
  Description of the artificial sequence suppression construct 2 p3300.1-Toc159-GFP-RNAi
aattcgtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagcaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagtctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgactcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttttggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctccatggcatgttctgcaggtcgactctagaggatccccgggtaccgagctcgaagatcttcgacgtcggaattcatggactcaaagtcggttactccagaaccaaccaaccccttctacgcttcttcggggcaatcaggaaaaacctatgcttctgttgtcgccgccgctgctgctgcagccgccgataaggaggatggtggtgctgtgagtagtgccaaggagttggattcctcatcggaggctgtgtctggtaattcggataaggttggagctgatgatttatctgactccgagaaggagaagccgaatttggtgggtgatgggaaggtttccgacgaggtggatggttctttaaaggaggattctactactcctgaggctactccgaagcctgaggtggtttctggtgagacaattggtgtagatgatgtttcatcgttatctccgaagccggaggctgtttctgatggtgtaggggttgtggaggagaataagaaggttaaggaggacgtggaggatattaaagacgatggtgagagtaagattgaaaatgggagtgttgatgttgatgtgaaacaggcttccacagatggggagagtgagaaagcttccaacacttgtcactactttctcttatggtgttcaatgcttttcaagatacccagatcatatgaaacggcatgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaccgaaaagagagaccacatggtccttcttgagtttgtaacagctgctgggattacacatggcatggatgaactatacaaacatgatgagctttaaggatccttaaagctcatcatgtttgtatagttcatccatgccatgtgtaatcccagcagctgttacaaactcaagaaggaccatgtggtctctcttttcggtgggatctttcgaaagggcagattgtgtggacaggtaatggttgtctggtaaaaggacagggccatcgccaattggagtattttgttgataatgatcagcgagttgcacgccgccgtcttcgatgttgtggcgggtcttgaagttggctttgatgccgttcttttgcttgtcggccatgatgtatacgttgtgggagttgtagttgtattccaacttgtggccgaggatgtttccgtcctccttgaaatcgattcccttaagctcgatcctgttgacgagggtgtctccctcaaacttgacttcagcacgtgtcttgtagttcccgtcgtccttgaagaagatggtcctctcctgcacgtatccctcaggcatggcgctcttgaagaagtcatgccgtttcatatgatctgggtatcttgaaaagcattgaacaccataagagaaagtagtgacaagtgttggaagctttctcactctccccatctgtggaagcctgtttcacatcaacatcaacactcccattttcaatcttactctcaccatcgtctttaatatcctccacgtcctccttaaccttcttattctcctccacaacccctacaccatcagaaacagcctccggcttcggagataacgatgaaacatcatctacaccaattgtctcaccagaaaccacctcaggcttcggagtagcctcaggagtagtagaatcctcctttaaagaaccatccacctcgtcggaaaccttcccatcacccaccaaattcggcttctccttctcggagtcagataaatcatcagctccaaccttatccgaattaccagacacagcctccgatgaggaatccaactccttggcactactcacagcaccaccatcctccttatcggcggctgcagcagcagcggcggcgacaacagaagcataggtttttcctgattgccccgaagaagcgtagaaggggttggttggttctggagtaaccgactttgagtccatg

SEQ ID NO: 123 - DNA, 36 bp, Artificial sequence
  Description of the artificial sequence oligonucleotide primer
ctcgaggaattcatggactcaaagtcggttactcca

SEQ ID NO: 124 - DNA, 40 bp, Artificial sequence
  Description of the artificial sequence oligonucleotide primer
ggatccataagcaagctttctcactctccccatctgtgga

SEQ ID NO: 125 - DNA, 26 bp, Artificial sequence
  Description of the artificial sequence oligonucleotide primer
aagcttccaacacttgtcactacttt

SEQ ID NO: 126 - DNA, 26 bp, Artificial sequence
  Description of the artificial sequence oligonucleotide primer
ggatccttaaagctcatcatgtttgt